SEQUENCE LISTING (77 sequences)

SEQ ID NO: 1 - DNA, 25 bp, synthetic construct
  source 1..25: mol_type = other DNA
caccggctgatctatcgcggtcgtc

SEQ ID NO: 2 - DNA, 25 bp, synthetic construct
  source 1..25: mol_type = other DNA
aaacgacgaccgcgatagatcagcc

SEQ ID NO: 3 - DNA, 25 bp, synthetic construct
  source 1..25: mol_type = other DNA
caccgtccatttcttgtggacggag

SEQ ID NO: 4 - DNA, 25 bp, synthetic construct
  source 1..25: mol_type = other DNA
aaacctccgtccacaagaaatggac

SEQ ID NO: 5 - DNA, 24 bp, synthetic construct
  source 1..24: mol_type = other DNA
accggaaggtttgggtctcgtggg

SEQ ID NO: 6 - DNA, 25 bp, synthetic construct
  source 1..25: mol_type = other DNA
aaaccccacgagacccaaaccttcc

SEQ ID NO: 7 - DNA, 25 bp, synthetic construct
  source 1..25: mol_type = other DNA
caccgggagaaggtttgggtctcgt

SEQ ID NO: 8 - DNA, 25 bp, synthetic construct
  source 1..25: mol_type = other DNA
aaacacgagacccaaaccttctccc

SEQ ID NO: 9 - DNA, 24 bp, synthetic construct
  source 1..24: mol_type = other DNA
tcccgaattttctgatgtgaatga

SEQ ID NO: 10 - DNA, 24 bp, synthetic construct
  source 1..24: mol_type = other DNA
aaactcattcacatcagaaaattc

SEQ ID NO: 11 - DNA, 25 bp, synthetic construct
  source 1..25: mol_type = other DNA
caccggggagaactccctatagcta

SEQ ID NO: 12 - DNA, 25 bp, synthetic construct
  source 1..25: mol_type = other DNA
aaactagctatagggagttctcccc

SEQ ID NO: 13 - DNA, 25 bp, synthetic construct
  source 1..25: mol_type = other DNA
caccgcggtgcagattcgaactgcg

SEQ ID NO: 14 - DNA, 25 bp, synthetic construct
  source 1..25: mol_type = other DNA
aaaccgcagttcgaatctgcaccgc

SEQ ID NO: 15 - DNA, 25 bp, synthetic construct
  source 1..25: mol_type = other DNA
caccgttgggcggcagcagtgacgc

SEQ ID NO: 16 - DNA, 25 bp, synthetic construct
  source 1..25: mol_type = other DNA
aaacgcgtcactgctgccgcccaac

SEQ ID NO: 17 - DNA, 20 bp, synthetic construct
  source 1..20: mol_type = other DNA
catgggcaagaagaagatcg

SEQ ID NO: 18 - DNA, 20 bp, synthetic construct
  source 1..20: mol_type = other DNA
tccaaggggatatccacaga

SEQ ID NO: 19 - DNA, 22 bp, synthetic construct
  source 1..22: mol_type = other DNA
ttgcagcttacgtttactggtt

SEQ ID NO: 20 - DNA, 23 bp, synthetic construct
  source 1..23: mol_type = other DNA
ggaacttgccttattggacaact

SEQ ID NO: 21 - DNA, 23 bp, synthetic construct
  source 1..23: mol_type = other DNA
ctgtaggaagcaacagagttacc

SEQ ID NO: 22 - DNA, 21 bp, synthetic construct
  source 1..21: mol_type = other DNA
cataggtccatgcacaagtgt

SEQ ID NO: 23 - DNA, 21 bp, synthetic construct
  source 1..21: mol_type = other DNA
tggcatttgctgaacgcattt

SEQ ID NO: 24 - DNA, 22 bp, synthetic construct
  source 1..22: mol_type = other DNA
tgcagccaggtctaattgtttt

SEQ ID NO: 25 - DNA, 20 bp, synthetic construct
  source 1..20: mol_type = other DNA
ctgtgcgagtgtaccggatg

SEQ ID NO: 26 - DNA, 21 bp, synthetic construct
  source 1..21: mol_type = other DNA
atccccacatgacttcctctt

SEQ ID NO: 27 - DNA, 21 bp, synthetic construct
  source 1..21: mol_type = other DNA
cagccagatgcaatcaatgcc

SEQ ID NO: 28 - DNA, 21 bp, synthetic construct
  source 1..21: mol_type = other DNA
tggaatcctgaacccacttct

SEQ ID NO: 29 - DNA, 21 bp, synthetic construct
  source 1..21: mol_type = other DNA
gatgaccagcgcgaccttatc

SEQ ID NO: 30 - DNA, 21 bp, synthetic construct
  source 1..21: mol_type = other DNA
gtgactgtcagtttgtccagc

SEQ ID NO: 31 - DNA, 21 bp, synthetic construct
  source 1..21: mol_type = other DNA
cctggagcttcttaacagcga

SEQ ID NO: 32 - DNA, 21 bp, synthetic construct
  source 1..21: mol_type = other DNA
tgtgtcgggttctgagtagag

SEQ ID NO: 33 - DNA, 23 bp, synthetic construct
  source 1..23: mol_type = other DNA
agtccctgtgctaggatttttca

SEQ ID NO: 34 - DNA, 22 bp, synthetic construct
  source 1..22: mol_type = other DNA
acataaactcgcctgattggtc

SEQ ID NO: 35 - DNA, 19 bp, synthetic construct
  source 1..19: mol_type = other DNA
cggggttggtgagagcttc

SEQ ID NO: 36 - DNA, 21 bp, synthetic construct
  source 1..21: mol_type = other DNA
aaccacctgacttcaatgctg

SEQ ID NO: 37 - DNA, 19 bp, synthetic construct
  source 1..19: mol_type = other DNA
gtctggccgtgagacgttt

SEQ ID NO: 38 - DNA, 23 bp, synthetic construct
  source 1..23: mol_type = other DNA
accataaaaggttcgagtcttcc

SEQ ID NO: 39 - DNA, 21 bp, synthetic construct
  source 1..21: mol_type = other DNA
aaagccgtcatcgtgaagaac

SEQ ID NO: 40 - DNA, 21 bp, synthetic construct
  source 1..21: mol_type = other DNA
gatagcggtcaattccagcca

SEQ ID NO: 41 - DNA, 21 bp, synthetic construct
  source 1..21: mol_type = other DNA
agccaccctctattagcagtt

SEQ ID NO: 42 - DNA, 22 bp, synthetic construct
  source 1..22: mol_type = other DNA
gccacaagagtgtctgtttgga

SEQ ID NO: 43 - DNA, 23 bp, synthetic construct
  source 1..23: mol_type = other DNA
taaaaacctggatcggaaccaaa

SEQ ID NO: 44 - DNA, 23 bp, synthetic construct
  source 1..23: mol_type = other DNA
gcattagcttcagatttacgggt

SEQ ID NO: 45 - DNA, 21 bp, synthetic construct
  source 1..21: mol_type = other DNA
ccaagtgctgccgtcattttc

SEQ ID NO: 46 - DNA, 21 bp, synthetic construct
  source 1..21: mol_type = other DNA
tccctatggccctcattctca

SEQ ID NO: 47 - DNA, 20 bp, synthetic construct
  source 1..20: mol_type = other DNA
aacctgaaactccccaggat

SEQ ID NO: 48 - DNA, 20 bp, synthetic construct
  source 1..20: mol_type = other DNA
tcatcgaattggcactcaaa

SEQ ID NO: 49 - DNA, 20 bp, synthetic construct
  source 1..20: mol_type = other DNA
gggcctctaaaggggtcaag

SEQ ID NO: 50 - DNA, 22 bp, synthetic construct
  source 1..22: mol_type = other DNA
tcaaacttcactccacaacgtc

SEQ ID NO: 51 - DNA, 20 bp, synthetic construct
  source 1..20: mol_type = other DNA
agtatggcagcaacgtcacg

SEQ ID NO: 52 - DNA, 22 bp, synthetic construct
  source 1..22: mol_type = other DNA
tccttttcccagtacaccacta

SEQ ID NO: 53 - DNA, 21 bp, synthetic construct
  source 1..21: mol_type = other DNA
ggccgatacaaagcaggagaa

SEQ ID NO: 54 - DNA, 20 bp, synthetic construct
  source 1..20: mol_type = other DNA
ggagttcatggcacaacgga

SEQ ID NO: 55 - DNA, 22 bp, synthetic construct
  source 1..22: mol_type = other DNA
gtgacgttgacatccgtaaaga

SEQ ID NO: 56 - DNA, 19 bp, synthetic construct
  source 1..19: mol_type = other DNA
gccggactcatcgtactcc

SEQ ID NO: 57 - DNA, 20 bp, synthetic construct
  source 1..20: mol_type = other DNA
tggagatgacggagaagatg

SEQ ID NO: 58 - DNA, 18 bp, synthetic construct
  source 1..18: mol_type = other DNA
ttggatggcaaaggcagt

SEQ ID NO: 59 - DNA, 19 bp, synthetic construct
  source 1..19: mol_type = other DNA
cctgtgtgatgcaggaacc

SEQ ID NO: 60 - DNA, 18 bp, synthetic construct
  source 1..18: mol_type = other DNA
tcacctcccaggcacaga

SEQ ID NO: 61 - DNA, 20 bp, synthetic construct
  source 1..20: mol_type = other DNA
aggcataacgcactaggttt

SEQ ID NO: 62 - DNA, 20 bp, synthetic construct
  source 1..20: mol_type = other DNA
agctggagtcacagaaggag

SEQ ID NO: 63 - RNA, 4495 nt, Homo sapiens
  source 1..4495: mol_type = genomic RNA
ggagcgaggccagggtagggtgagcggcctccgaagcggagcggggctctgaggagacactttttttttcctccctccttccctcctctcctcctcccttcccttcccctctcctcccctctctcctccttcccccctcggtccgccggagcctgctggggcgagcggttggtattgcaggcgcttgctctccggggccgcccggcgggtagctggcggggggaggaggcaggaaccgcgatggcgcctcagaagcacggcggtgggggagggggcggctcggggcccagcgcggggtccgggggaggcggcttcgggggttcggcggcggtggcggcggcgacggcttcgggcggcaaatccggcggcgggagctgtggagggggtggcagttactcggcctcctcctcctcctccgcggcggcagcggcgggggctgcggtgttaccggtgaagaagccgaaaatggagcacgtccaggctgaccacgagcttttcctccaggcctttgagaagccaacacagatctatagatttcttcgaactcggaatctcatagcaccaatatttttgcacagaactcttacttacatgtctcatcgaaactccagaacaaacatcaaaaggaaaacatttaaagttgatgatatgttatcaaaagtagagaaaatgaaaggagagcaagaatctcatagcttgtcagctcatttgcagcttacgtttactggtttcttccacaaaaatgataagccatcaccaaactcagaaaatgaacaaaattctgttaccctggaagtcctgcttgtgaaagtttgccacaaaaaaagaaaggatgtaagttgtccaataaggcaagttcccacaggtaaaaagcaggtgcctttgaatcctgacctcaatcaaacaaaacccggaaatttcccgtcccttgcagtttccagtaatgaatttgaacctagtaacagccatatggtgaagtcttactcgttgctatttagagtgactcgtccaggaagaagagagtttaatggaatgattaatggagaaaccaatgaaaatattgatgtcaatgaagagcttccagccagaagaaaacgaaatcgtgaggatggggaaaagacatttgttgcacaaatgacagtatttgataaaaacaggcgcttacagcttttagatggggaatatgaagtagccatgcaggaaatggaagaatgtccaataagcaagaaaagagcaacatgggagactattcttgatgggaagaggctgcctccattcgaaacattttctcagggacctacgttgcagttcactcttcgttggacaggagagaccaatgataaatctacggctcctattgccaaacctcttgccactagaaattcagagagtctccatcaggaaaacaagcctggttcagttaaacctactcaaactattgctgttaaagaatcattgactacagatctacaaacaagaaaagaaaaggatactccaaatgaaaaccgacaaaaattaagaatattttatcagtttctctataacaacaatacaaggcaacaaactgaagcaagagatgacctgcattgcccttggtgtactctgaactgccgcaaactttatagtttactcaagcatcttaaactctgccatagcagatttatcttcaactatgtttatcatccaaaaggtgctaggatagatgtttctatcaatgagtgttatgatggctcctatgcaggaaatcctcaggatattcatcgccaacctggatttgcttttagtcgcaacggaccagttaagagaacacctatcacacatattcttgtgtgcaggccaaaacgaacaaaagcaagcatgtctgaatttcttgaatctgaagatggggaagtagaacagcaaagaacatatagtagtggccacaatcgtctgtatttccatagtgatacctgcttacctctccgtccacaagaaatggaagtagatagtgaagatgaaaaggatcctgaatggctaagagaaaaaaccattacacaaattgaagagttttctgatgttaatgaaggagagaaagaagtgatgaaactctggaatctccatgtcatgaagcatgggtttattgctgacaatcaaatgaatcatgcctgtatgctgtttgtagaaaattatggacagaaaataattaagaagaatttatgtcgaaacttcatgcttcatctagtcagcatgcatgactttaatcttattagcataatgtcaatagataaagctgttaccaagctccgtgaaatgcagcaaaaattagaaaagggggaatctgcttcccctgcaaacgaagaaataactgaagaacaaaatgggacagcaaatggatttagtgaaattaactcaaaagagaaagctttggaaacagatagtgtctcaggggtttcaaaacagagcaaaaaacaaaaactctgaaaagctctaaccccatgttatggacaaacactgaaattacattttagggaattcatcctctaagaattatgtttttgtttttaatcatatgttccaaacaggcactgttagatgaagtaaatgatttcaacaaggatatttgtatcagggttctacttcacttcattatgcagcattacatgtatatcacttttattgatgtcattaaaacattctgtactttaagcatgaaaagcaatatttcaaagtatttttaaactcaacaaatgtcatcaaatatgttgaattgatctagaaattatttcatatataaatcagaatttttttgcatttatgaacggctgtttttctactttgtaattgtgagacattttcttggggagggaaaattggaatggttcccttttttagaaattgaagtggtcttcatatgtcaactacagaaaaggaaaaaaatagaaattgaaggatttttatgaaattatattgcattactatttgcagtcaaactttgatccttgtttttgaaatcatttgtcaattcggaatgaaaaattataatgtaattttacattacataagttccttttacaattaaaaaatagcacttcttcatcttatgcctgtttgagaagatattaaattttcacattgttgacagtgaaatgctatgttggtttataagattacagaccatttgttttcatgtggataattttagtgcattgctcacccggtatgttttttttttttaacttgaacattttgcttgttttgtttttcttttttaattagataatcacacggaaaattaagctgttcatatctttaaattaggattgcaaaccaaggaaagaacgcatttgagattttaagatgtcacttataaggggagaagtgttcttaaaaagtcaaccagaaaactgttatgccttttatttgtttgcaaggatgtctttgtaatgtgtttcatgaatagaatatccaatagagataagctgacttgaatcattttgagcaattttgccctgtgttatatgtgtttcacgcacatatttgcagttggattttctccaacagaaagtggattcactactggcacattaacaagcaccaataggtttttattccaactccgagcactgtggttgagtaacatcacctcaattttttattatccttaaagatattgcattttcatattctttatttataaaggatcaatgctgctgtaaatacaggtatttttaattttaaaatttcattccaccaccatcagatgcagttccctattttgtttaatgaagggatatataagctttctaatggtgtcttcagaaatttataaaatgtaaatactgatttgactggtctttaagatgtgtttaactgtgaggctatttaacgaatagtgtggatgtgatttgtcatccagtattaagttcttagtcattgatttttgtgtttaaaaaaaaataggaaagagggaaactgcagctttcattacagattccttgattggtaagctctccaaatgatgagttctagtaaactctgatttttgcctctggatagtagatctcgagcgtttatctcgggctttaatttgctaaagctgtgcacatatgtaaaaaaaaaaaaaaaaagattattttaggggagatgtaggtgtagaattattgcttatgtcatttcttaagcagttatgctcttaatgcttaaaagaaggctagcattgtttgcacaaaaagttggtgattcccaccccaaatagtaataaaattacttctgttgagtaaactttttatgtcatcgtaaaagctgaaaaaatccctttgtttctatttataaaaaaagtgcttttctatatgtacccttgataacagattttgaagaaatcctgtaagatgataaagcatttgaatggtacagtagatgtaaaaaaaattcagtttaaaagaacatttgtttttacattaaatgtttatttgaaatcaaatgattttgtacataaagttcaataatataaaagctg

SEQ ID NO: 64 - RNA, 4426 nt, Homo sapiens
  source 1..4426: mol_type = genomic RNA
ggagcgaggccagggtagggtgagcggcctccgaagcggagcggggctctgaggagacactttttttttcctccctccttccctcctctcctcctcccttcccttcccctctcctcccctctctcctccttcccccctcggtccgccggagcctgctggggcgagcggttggtattgcaggcgcttgctctccggggccgcccggcgggtagctggcggggggaggaggcaggaaccgcgatggcgcctcagaagcacggcggtgggggagggggcggctcggggcccagcgcggggtccgggggaggcggcttcgggggttcggcggcggtggcggcggcgacggcttcgggcggcaaatccggcggcgggagctgtggagggggtggcagttactcggcctcctcctcctcctccgcggcggcagcggcgggggctgcggtgttaccggtgaagaagccgaaaatggagcacgtccaggctgaccacgagcttttcctccaggcctttgagaagccaacacagatctatagatttcttcgaactcggaatctcatagcaccaatatttttgcacagaactcttacttacatgtctcatcgaaactccagaacaaacatcaaaagcttgtcagctcatttgcagcttacgtttactggtttcttccacaaaaatgataagccatcaccaaactcagaaaatgaacaaaattctgttaccctggaagtcctgcttgtgaaagtttgccacaaaaaaagaaaggatgtaagttgtccaataaggcaagttcccacaggtaaaaagcaggtgcctttgaatcctgacctcaatcaaacaaaacccggaaatttcccgtcccttgcagtttccagtaatgaatttgaacctagtaacagccatatggtgaagtcttactcgttgctatttagagtgactcgtccaggaagaagagagtttaatggaatgattaatggagaaaccaatgaaaatattgatgtcaatgaagagcttccagccagaagaaaacgaaatcgtgaggatggggaaaagacatttgttgcacaaatgacagtatttgataaaaacaggcgcttacagcttttagatggggaatatgaagtagccatgcaggaaatggaagaatgtccaataagcaagaaaagagcaacatgggagactattcttgatgggaagaggctgcctccattcgaaacattttctcagggacctacgttgcagttcactcttcgttggacaggagagaccaatgataaatctacggctcctattgccaaacctcttgccactagaaattcagagagtctccatcaggaaaacaagcctggttcagttaaacctactcaaactattgctgttaaagaatcattgactacagatctacaaacaagaaaagaaaaggatactccaaatgaaaaccgacaaaaattaagaatattttatcagtttctctataacaacaatacaaggcaacaaactgaagcaagagatgacctgcattgcccttggtgtactctgaactgccgcaaactttatagtttactcaagcatcttaaactctgccatagcagatttatcttcaactatgtttatcatccaaaaggtgctaggatagatgtttctatcaatgagtgttatgatggctcctatgcaggaaatcctcaggatattcatcgccaacctggatttgcttttagtcgcaacggaccagttaagagaacacctatcacacatattcttgtgtgcaggccaaaacgaacaaaagcaagcatgtctgaatttcttgaatctgaagatggggaagtagaacagcaaagaacatatagtagtggccacaatcgtctgtatttccatagtgatacctgcttacctctccgtccacaagaaatggaagtagatagtgaagatgaaaaggatcctgaatggctaagagaaaaaaccattacacaaattgaagagttttctgatgttaatgaaggagagaaagaagtgatgaaactctggaatctccatgtcatgaagcatgggtttattgctgacaatcaaatgaatcatgcctgtatgctgtttgtagaaaattatggacagaaaataattaagaagaatttatgtcgaaacttcatgcttcatctagtcagcatgcatgactttaatcttattagcataatgtcaatagataaagctgttaccaagctccgtgaaatgcagcaaaaattagaaaagggggaatctgcttcccctgcaaacgaagaaataactgaagaacaaaatgggacagcaaatggatttagtgaaattaactcaaaagagaaagctttggaaacagatagtgtctcaggggtttcaaaacagagcaaaaaacaaaaactctgaaaagctctaaccccatgttatggacaaacactgaaattacattttagggaattcatcctctaagaattatgtttttgtttttaatcatatgttccaaacaggcactgttagatgaagtaaatgatttcaacaaggatatttgtatcagggttctacttcacttcattatgcagcattacatgtatatcacttttattgatgtcattaaaacattctgtactttaagcatgaaaagcaatatttcaaagtatttttaaactcaacaaatgtcatcaaatatgttgaattgatctagaaattatttcatatataaatcagaatttttttgcatttatgaacggctgtttttctactttgtaattgtgagacattttcttggggagggaaaattggaatggttcccttttttagaaattgaagtggtcttcatatgtcaactacagaaaaggaaaaaaatagaaattgaaggatttttatgaaattatattgcattactatttgcagtcaaactttgatccttgtttttgaaatcatttgtcaattcggaatgaaaaattataatgtaattttacattacataagttccttttacaattaaaaaatagcacttcttcatcttatgcctgtttgagaagatattaaattttcacattgttgacagtgaaatgctatgttggtttataagattacagaccatttgttttcatgtggataattttagtgcattgctcacccggtatgttttttttttttaacttgaacattttgcttgttttgtttttcttttttaattagataatcacacggaaaattaagctgttcatatctttaaattaggattgcaaaccaaggaaagaacgcatttgagattttaagatgtcacttataaggggagaagtgttcttaaaaagtcaaccagaaaactgttatgccttttatttgtttgcaaggatgtctttgtaatgtgtttcatgaatagaatatccaatagagataagctgacttgaatcattttgagcaattttgccctgtgttatatgtgtttcacgcacatatttgcagttggattttctccaacagaaagtggattcactactggcacattaacaagcaccaataggtttttattccaactccgagcactgtggttgagtaacatcacctcaattttttattatccttaaagatattgcattttcatattctttatttataaaggatcaatgctgctgtaaatacaggtatttttaattttaaaatttcattccaccaccatcagatgcagttccctattttgtttaatgaagggatatataagctttctaatggtgtcttcagaaatttataaaatgtaaatactgatttgactggtctttaagatgtgtttaactgtgaggctatttaacgaatagtgtggatgtgatttgtcatccagtattaagttcttagtcattgatttttgtgtttaaaaaaaaataggaaagagggaaactgcagctttcattacagattccttgattggtaagctctccaaatgatgagttctagtaaactctgatttttgcctctggatagtagatctcgagcgtttatctcgggctttaatttgctaaagctgtgcacatatgtaaaaaaaaaaaaaaaaagattattttaggggagatgtaggtgtagaattattgcttatgtcatttcttaagcagttatgctcttaatgcttaaaagaaggctagcattgtttgcacaaaaagttggtgattcccaccccaaatagtaataaaattacttctgttgagtaaactttttatgtcatcgtaaaagctgaaaaaatccctttgtttctatttataaaaaaagtgcttttctatatgtacccttgataacagattttgaagaaatcctgtaagatgataaagcatttgaatggtacagtagatgtaaaaaaaattcagtttaaaagaacatttgtttttacattaaatgtttatttgaaatcaaatgattttgtacataaagttcaataatataaaagctg

SEQ ID NO: 65 - RNA, 2088 nt, Homo sapiens
  source 1..2088: mol_type = genomic RNA
attccacagactttcgctccctagcagcgggtcggagatcgaaggaacgggccaattgcggctgaaacgtctttggaaggaggaagggggtgagggagcatccctttgagtttcgcctcttctcgaggcggtggtgggaagggagacatacttaatactgccctcttaatccaacggaccttacatcgtgtagactgccgggagggcggcgggaaaagggcaagacgggagttggggaagggaaggagccaggaagccgcgcgggagggcgcgcgcgcgcgcccctttttcagcagtgtggcggggtcgcacgcacgcccgcctcggcggctgggcgcgatttgcgacagtggggggggcggtggaggtggcggcggcagcggcaactttgcggcaagctcgggccgggcttgcttgacggcggtgtggcggaggccccgccccaggcggcaggaacctggagggaggcggaggaatatgtccgagagggaagtgtcgactgcgccggcgggaacagacatgcctgcggccaagaagcagaagctgagcagtgacgagaacagcaatccagacctctctggagacgagaatgatgacgctgtcagtatagaaagtggtacaaacactgaacgccctgatacacctacaaacacgccaaatgcacctggaaggaaaagttggggaaagggaaaatggaagtcaaagaaatgcaaatattctttcaaatgtgtaaatagtctcaaggaagatcataaccaaccattgtttggagttcagtttaactggcacagtaaagaaggagatccattagtgtttgcaactgtaggaagcaacagagttaccttgtatgaatgtcattcacaaggagaaatccggttgttgcaatcttacgtggatgctgatgctgatgaaaacttttacacttgtgcatggacctatgatagcaatacgagccatcctctgctggctgtagctggatctagaggcataattaggataataaatcctataacaatgcagtgtataaagcactatgttggccatggaaatgctatcaatgagctgaaattccatccaagagatccaaatcttctcctgtcagtaagtaaagatcatgctttacgattatggaatatccagacggacactctggtggcaatatttggaggcgtagaagggcacagagatgaagttctaagtgctgattatgatcttttgggtgaaaaaataatgtcctgtggtatggatcattctcttaaactttggaggatcaattcaaagagaatgatgaatgcaattaaggaatcttatgattataatccaaataaaactaacaggccatttatttctcagaaaatccattttcctgatttttctaccagagacatacataggaattatgttgattgtgtgcgatggttaggcgatttgatactttctaagtcttgtgaaaatgccattgtgtgctggaaacctggcaagatggaagatgatatagataaaattaaacccagtgaatctaatgtgactattcttgggcgatttgattacagccagtgtgacatttggtacatgaggttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataaaatacttttgcctaatcaaaattagagtgtgtttgttgtctgtgtaaaatagaattaatgtatcttgctagtaagggcacgtagagcatttagagttgtctttcagcattcaatcaggctgagctgaatgtagtgatgtttacattgtttacattctttgtactgtcttcctgctcagactctactgcttttaataaaaatttatttttgtaaagctgtgtgtttagttactttcattgtggtgaaaaaaagttaaaagtaataaaattatgccttatcttttta

SEQ ID NO: 66 - RNA, 2163 nt, Homo sapiens
  source 1..2163: mol_type = genomic RNA
attccacagactttcgctccctagcagcgggtcggagatcgaaggaacgggccaattgcggctgaaacgtctttggaaggaggaagggggtgagggagcatccctttgagtttcgcctcttctcgaggcggtggtgggaagggagacatacttaatactgccctcttaatccaacggaccttacatcgtgtagactgccgggagggcggcgggaaaagggcaagacgggagttggggaagggaaggagccaggaagccgcgcgggagggcgcgcgcgcgcgcccctttttcagcagtgtggcggggtcgcacgcacgcccgcctcggcggctgggcgcgatttgcgacagtggggggggcggtggaggtggcggcggcagcggcaactttgcggcaagctcgggccgggcttgcttgacggcggtgtggcggaggccccgccccaggcggcaggaacctggagggaggcggaggaatatgtccgagagggaagtgtcgactgcgccggcgggaacagacatgcctgcggccaagaagcagaagctgagcagtgacgagaacagcaatccagacctctctggagacgagaatgatgacgctgtcagtatagaaagtggtacaaacactgaacgccctgatacacctacaaacacgccaaatgcacctggaaggaaaagttggggaaagggaaaatggaagtcaaagaaatgcaaatattctttcaaatgtgtaaatagtctcaaggaagatcataaccaaccattgtttggagttcagtttaactggcacagtaaagaaggagatccattagtgtttgcaactgtaggaagcaacagagttaccttgtatgaatgtcattcacaaggagaaatccggttgttgcaatcttacgtggatgctgatgctgatgaaaacttttacacttgtgcatggacctatgatagcaatacgagccatcctctgctggctgtagctggatctagaggcataattaggataataaatcctataacaatgcagtgtataaagcactatgttggccatggaaatgctatcaatgagctgaaattccatccaagagatccaaatcttctcctgtcagtaagtaaagatcatgctttacgattatggaatatccagacggacactctggtggcaatatttggaggcgtagaagggcacagagatgaagttctaagtgctgattatgatcttttgggtgaaaaaataatgtcctgtggtatggatcattctcttaaactttggaggatcaattcaaagagaatgatgaatgcaattaaggaatcttatgattataatccaaataaaactaacaggccatttatttctcagaaaatccattttcctgatttttctaccagagacatacataggaattatgttgattgtgtgcgatggttaggcgatttgatactttctaagagtggccgtgccattttacattcccaccagcaatgtatgagagatccagtgtctccgaatcttcgccagcatttgtcttgtgaaaatgccattgtgtgctggaaacctggcaagatggaagatgatatagataaaattaaacccagtgaatctaatgtgactattcttgggcgatttgattacagccagtgtgacatttggtacatgaggttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataaaatacttttgcctaatcaaaattagagtgtgtttgttgtctgtgtaaaatagaattaatgtatcttgctagtaagggcacgtagagcatttagagttgtctttcagcattcaatcaggctgagctgaatgtagtgatgtttacattgtttacattctttgtactgtcttcctgctcagactctactgcttttaataaaaatttatttttgtaaagctgtgtgtttagttactttcattgtggtgaaaaaaagttaaaagtaataaaattatgccttatcttttta

SEQ ID NO: 67 - RNA, 1848 nt, Homo sapiens
  source 1..1848: mol_type = genomic RNA
attccacagactttcgctccctagcagcgggtcggagatcgaaggaacgggccaattgcggctgaaacgtctttggaaggaggaagggggtgagggagcatccctttgagtttcgcctcttctcgaggcggtggtgggaagggagacatacttaatactgccctcttaatccaacggaccttacatcgtgtagactgccgggagggcggcgggaaaagggcaagacgggagttggggaagggaaggagccaggaagccgcgcgggagggcgcgcgcgcgcgcccctttttcagcagtgtggcggggtcgcacgcacgcccgcctcggcggctgggcgcgatttgcgacagtggggggggcggtggaggtggcggcggcagcggcaactttgcggcaagctcgggccgggcttgcttgacggcggtgtggcggaggccccgccccaggcggcaggaacctggagggaggcggaggaatatgtccgagagggaagtgtcgactgcgccggcgggaacagacatgcctgcggccaagaagcagaagctgagcagtgacgagaacagcaatccagacctctctggagacgagaatgatgacgctgtcagtatagaaagtggtacaaacactgaacgccctgatacacctacaaacacgccaaatgcacctggaaggaaaagttggggaaagggaaaatggaagtcaaagaaatgcaaatattctttcaaatgtgtaaatagtctcaaggaagatcataaccaaccattgtttggagttcagtttaactggcacagtaaagaaggagatccattagtgtttgcaactgtaggaagcaacagagttaccttgtatgaatgtcattcacaaggagaaatccggttgttgcaatcttacgtggatgctgatgctgatgaaaacttttacacttgtgcatggacctatgatagcaatacgagccatcctctgctggctgtagctggatctagaggcataattaggataataaatcctataacaatgcagtgtataaagcactatgttggccatggaaatgctatcaatgagctgaaattccatccaagagatccaaatcttctcctgtcagtaagtaaagatcatgctttacgattatggaatatccagacggacactctggtggcaatatttggaggcgtagaagggcacagagatgaagttctaagtgcttcttgtgaaaatgccattgtgtgctggaaacctggcaagatggaagatgatatagataaaattaaacccagtgaatctaatgtgactattcttgggcgatttgattacagccagtgtgacatttggtacatgaggttttctatggatttctggcaaaagatgcttgcattgggcaatcaagttggcaaactttatgtttgggatttagaagtagaagatcctcataaagccaaatgtacaacactgactcatcataaatgtggtgctgctattcgacaaaccagttttagcagggatagcagcattcttatagctgtttgtgatgatgccagtatttggcgctgggatcgacttcgataaaatacttttgcctaatcaaaattagagtgtgtttgttgtctgtgtaaaatagaattaatgtatcttgctagtaagggcacgtagagcatttagagttgtctttcagcattcaatcaggctgagctgaatgtagtgatgtttacattgtttacattctttgtactgtcttcctgctcagactctactgcttttaataaaaatttatttttgtaaagctgtgtgtttagttactttcattgtggtgaaaaaaagttaaaagtaataaaattatgccttatcttttta

SEQ ID NO: 68 - RNA, 4542 nt, Homo sapiens
  source 1..4542: mol_type = genomic RNA
agcagcgggacccgcggctcgggatggaggattacagcaagatggaaataccaaatccccctacctccaaatgtatcacttactggaaaagaaaagtgaaatctgaatacatgcgacttcgacaacttaaacggcttcaggcaaatatgggtgcaaaggctttgtatgtggcaaattttgcaaaggttcaagaaaaaacccagatcctcaatgaagaatggaagaagcttcgtgtccaacctgttcagtcaatgaagcctgtgagtggacacccttttctcaaaaagtgtaccatagagagcattttcccgggatttgcaagccaacatatgttaatgaggtcactgaacacagttgcattggttcccatcatgtattcctggtcccctctccaacagaactttatggtagaagatgagacggttttgtgcaatattccctacatgggagatgaagtgaaagaagaagatgagacttttattgaggagctgatcaataactatgatgggaaagtccatggtgaagaagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggcaacaaaaagagttccaagaaacagttcccaaatgacatgatcttcagtgcaattgcctcaatgttccctgagaatggtgtcccagatgacatgaaggagaggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcacccttttcatgccacccctaatgtatataaacgcaagaataaagaaatcaagattgaaccagaaccatgtggcacagactgcttccttttgctggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtctttcagtttgcagtcaaagaatcacttatcctgaagctgccaacagatgagctcatgaacccctcacagaagaagaaaagaaagcacagattgtgggctgcacactgcaggaagattcagctgaagaaagataactcttccacacaagtgtacaactaccaaccctgcgaccacccagaccgcccctgtgacagcacctgcccctgcatcatgactcagaatttctgtgagaagttctgccagtgcaacccagactgtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaagcacctgctgctggccccctctgatgtggccggatggggcaccttcataaaggagtctgtgcagaagaacgaattcatttctgaatactgtggtgagctcatctctcaggatgaggctgatcgacgcggaaaggtctatgacaaatacatgtccagcttcctcttcaacctcaataatgattttgtagtggatgctactcggaaaggaaacaaaattcgatttgcaaatcattcagtgaatcccaactgttatgccaaagtggtcatggtgaatggagaccatcggattgggatctttgccaagagggcaattcaagctggcgaagagctcttctttgattacaggtacagccaagctgatgctctcaagtacgtggggatcgagagggagaccgacgtcctttagccctcccaggccccacggcagcacttatggtagcggcactgtcttggctttcgtgctcacaccactgctgctcgagtctcctgcactgtgtctcccacactgagaaaccccccaacccactccctctgtagtgaggcctctgccatgtccagagggcacaaaactgtctcaatgagaggggagacagaggcagctagggcttggtctcccaggacagagagttacagaaatgggagactgtttctctggcctcagaagaagcgagcacaggctggggtggatgacttatgcgtgatttcgtgtcggctccccaggctgtggcctcaggaatcaacttaggcagttcccaacaagcgctagcctgtaattgtagctttccacatcaagagtccttatgttattgggatgcaggcaaacctctgtggtcctaagacctggagaggacaggctaagtgaagtgtggtccctggagcctacaagtggtctgggttagaggcgagcctggcaggcagcacagactgaactcagaggtagacaggtcaccttactacctcctccctcgtggcagggctcaaactgaaagagtgtgggttctaagtacaggcattcaaggctgggggaaggaaagctacgccatccttccttagccagagagggagaaccagccagatgatagtagttaaactgctaagcttgggcccaggaggctttgagaaagccttctctgtgtactctggagatagatggagaagtgttttcagattcctgggaacagacaccagtgctccagctcctccaaagttctggcttagcagctgcaggcaagcattatgctgctattgaagaagcattaggggtatgcctggcaggtgtgagcatcctggctcgctggatttgtgggtgttttcaggccttccattccccatagaggcaaggcccaatggccagtgttgcttatcgcttcagggtaggtgggcacaggcttggactagagaggagaaagattggtgtaatctgctttcctgtctgtagtgcctgctgtttggaaagggtgagttagaatatgttccaaggttggtgaggggctaaattgcacgcgtttaggctggcaccccgtgtgcagggcacactggcagagggtatctgaagtgggagaagaagcaggtagaccacctgtcccaggctgtggtgccaccctctctggcattcatgcagagcaaagcactttaaccatttcttttaaaaggtctatagattggggtagagtttggcctaaggtctctagggtccctgcctaaatcccactcctgagggagggggaagaagagagggtgggagattctcctccagtcctgtctcatctcctgggagaggcagacgagtgagtttcacacagaagaatttcatgtgaatggggccagcaagagctgccctgtgtccatggtgggtgtgccgggctggctgggaacaaggagcagtatgttgagtagaaagggtgtgggcgggtatagattggcctgggagtgttacagtagggagcaggcttctcccttctttctgggactcagagccccgcttcttcccactccacttgttgtcccatgaaggaagaagtggggttcctcctgacccagctgcctcttacggtttggtatgggacatgcacacacactcacatgctctcactcaccacactggagggcacacacgtaccccgcacccagcaactcctgacagaaagctcctcccacccaaatgggccaggccccagcatgatcctgaaatctgcatccgccgtggtttgtattcattgtgcatatcagggataccctcaagctggactgtgggttccaaattactcatagaggagaaaaccagagaaagatgaagaggaggagttaggtctatttgaaatgccaggggctcgctgtgaggaataggtgaaaaaaaacttttcaccagcctttgagagactagactgaccccacccttccttcagtgagcagaatcactgtggtcagtctcctgtcccagcttcagttcatgaatactcctgttcctccagtttcccatcctttgtccctgctgtcccccacttttaaagatgggtctcaacccctccccaccacgtcatgatggatggggcaaggtggtggggactaggggagcctggtatacatgcggcttcattgccaataaatttcatgcactttaaagtcctgtggcttgtgacctcttaataaagtgttagaatcca

SEQ ID NO: 69 - RNA, 4513 nt, Homo sapiens
  source 1..4513: mol_type = genomic RNA
agcagcgggacccgcggctcgggatggaggctggacacctgttctgctgttgtgtcctgccattctcctgaagaacagaggcacactgtaaaacccaacacttccccttgcattctataagattacagcaagatggaaataccaaatccccctacctccaaatgtatcacttactggaaaagaaaagtgaaatctgaatacatgcgacttcgacaacttaaacggcttcaggcaaatatgggtgcaaaggctttgtatgtggcaaattttgcaaaggttcaagaaaaaacccagatcctcaatgaagaatggaagaagcttcgtgtccaacctgttcagtcaatgaagcctgtgagtggacacccttttctcaaaaaggtagaagatgagacggttttgtgcaatattccctacatgggagatgaagtgaaagaagaagatgagacttttattgaggagctgatcaataactatgatgggaaagtccatggtgaagaagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggcaacaaaaagagttccaagaaacagttcccaaatgacatgatcttcagtgcaattgcctcaatgttccctgagaatggtgtcccagatgacatgaaggagaggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcacccttttcatgccacccctaatgtatataaacgcaagaataaagaaatcaagattgaaccagaaccatgtggcacagactgcttccttttgctggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtctttcagtttgcagtcaaagaatcacttatcctgaagctgccaacagatgagctcatgaacccctcacagaagaagaaaagaaagcacagattgtgggctgcacactgcaggaagattcagctgaagaaagataactcttccacacaagtgtacaactaccaaccctgcgaccacccagaccgcccctgtgacagcacctgcccctgcatcatgactcagaatttctgtgagaagttctgccagtgcaacccagactgtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaagcacctgctgctggccccctctgatgtggccggatggggcaccttcataaaggagtctgtgcagaagaacgaattcatttctgaatactgtggtgagctcatctctcaggatgaggctgatcgacgcggaaaggtctatgacaaatacatgtccagcttcctcttcaacctcaataatgattttgtagtggatgctactcggaaaggaaacaaaattcgatttgcaaatcattcagtgaatcccaactgttatgccaaagtggtcatggtgaatggagaccatcggattgggatctttgccaagagggcaattcaagctggcgaagagctcttctttgattacaggtacagccaagctgatgctctcaagtacgtggggatcgagagggagaccgacgtcctttagccctcccaggccccacggcagcacttatggtagcggcactgtcttggctttcgtgctcacaccactgctgctcgagtctcctgcactgtgtctcccacactgagaaaccccccaacccactccctctgtagtgaggcctctgccatgtccagagggcacaaaactgtctcaatgagaggggagacagaggcagctagggcttggtctcccaggacagagagttacagaaatgggagactgtttctctggcctcagaagaagcgagcacaggctggggtggatgacttatgcgtgatttcgtgtcggctccccaggctgtggcctcaggaatcaacttaggcagttcccaacaagcgctagcctgtaattgtagctttccacatcaagagtccttatgttattgggatgcaggcaaacctctgtggtcctaagacctggagaggacaggctaagtgaagtgtggtccctggagcctacaagtggtctgggttagaggcgagcctggcaggcagcacagactgaactcagaggtagacaggtcaccttactacctcctccctcgtggcagggctcaaactgaaagagtgtgggttctaagtacaggcattcaaggctgggggaaggaaagctacgccatccttccttagccagagagggagaaccagccagatgatagtagttaaactgctaagcttgggcccaggaggctttgagaaagccttctctgtgtactctggagatagatggagaagtgttttcagattcctgggaacagacaccagtgctccagctcctccaaagttctggcttagcagctgcaggcaagcattatgctgctattgaagaagcattaggggtatgcctggcaggtgtgagcatcctggctcgctggatttgtgggtgttttcaggccttccattccccatagaggcaaggcccaatggccagtgttgcttatcgcttcagggtaggtgggcacaggcttggactagagaggagaaagattggtgtaatctgctttcctgtctgtagtgcctgctgtttggaaagggtgagttagaatatgttccaaggttggtgaggggctaaattgcacgcgtttaggctggcaccccgtgtgcagggcacactggcagagggtatctgaagtgggagaagaagcaggtagaccacctgtcccaggctgtggtgccaccctctctggcattcatgcagagcaaagcactttaaccatttcttttaaaaggtctatagattggggtagagtttggcctaaggtctctagggtccctgcctaaatcccactcctgagggagggggaagaagagagggtgggagattctcctccagtcctgtctcatctcctgggagaggcagacgagtgagtttcacacagaagaatttcatgtgaatggggccagcaagagctgccctgtgtccatggtgggtgtgccgggctggctgggaacaaggagcagtatgttgagtagaaagggtgtgggcgggtatagattggcctgggagtgttacagtagggagcaggcttctcccttctttctgggactcagagccccgcttcttcccactccacttgttgtcccatgaaggaagaagtggggttcctcctgacccagctgcctcttacggtttggtatgggacatgcacacacactcacatgctctcactcaccacactggagggcacacacgtaccccgcacccagcaactcctgacagaaagctcctcccacccaaatgggccaggccccagcatgatcctgaaatctgcatccgccgtggtttgtattcattgtgcatatcagggataccctcaagctggactgtgggttccaaattactcatagaggagaaaaccagagaaagatgaagaggaggagttaggtctatttgaaatgccaggggctcgctgtgaggaataggtgaaaaaaaacttttcaccagcctttgagagactagactgaccccacccttccttcagtgagcagaatcactgtggtcagtctcctgtcccagcttcagttcatgaatactcctgttcctccagtttcccatcctttgtccctgctgtcccccacttttaaagatgggtctcaacccctccccaccacgtcatgatggatggggcaaggtggtggggactaggggagcctggtatacatgcggcttcattgccaataaatttcatgcactttaaagtcctgtggcttgtgacctcttaataaagtgttagaatcca

SEQ ID NO: 70 - RNA, 4633 nt, Homo sapiens
  source 1..4633: mol_type = genomic RNA
agcagcgggacccgcggctcgggatggaggctggacacctgttctgctgttgtgtcctgccattctcctgaagaacagaggcacactgtaaaacccaacacttccccttgcattctataagattacagcaagatggaaataccaaatccccctacctccaaatgtatcacttactggaaaagaaaagtgaaatctgaatacatgcgacttcgacaacttaaacggcttcaggcaaatatgggtgcaaaggctttgtatgtggcaaattttgcaaaggttcaagaaaaaacccagatcctcaatgaagaatggaagaagcttcgtgtccaacctgttcagtcaatgaagcctgtgagtggacacccttttctcaaaaagtgtaccatagagagcattttcccgggatttgcaagccaacatatgttaatgaggtcactgaacacagttgcattggttcccatcatgtattcctggtcccctctccaacagaactttatggtagaagatgagacggttttgtgcaatattccctacatgggagatgaagtgaaagaagaagatgagacttttattgaggagctgatcaataactatgatgggaaagtccatggtgaagaagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggcaacaaaaagagttccaagaaacagttcccaaatgacatgatcttcagtgcaattgcctcaatgttccctgagaatggtgtcccagatgacatgaaggagaggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcacccttttcatgccacccctaatgtatataaacgcaagaataaagaaatcaagattgaaccagaaccatgtggcacagactgcttccttttgctggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtctttcagtttgcagtcaaagaatcacttatcctgaagctgccaacagatgagctcatgaacccctcacagaagaagaaaagaaagcacagattgtgggctgcacactgcaggaagattcagctgaagaaagataactcttccacacaagtgtacaactaccaaccctgcgaccacccagaccgcccctgtgacagcacctgcccctgcatcatgactcagaatttctgtgagaagttctgccagtgcaacccagactgtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaagcacctgctgctggccccctctgatgtggccggatggggcaccttcataaaggagtctgtgcagaagaacgaattcatttctgaatactgtggtgagctcatctctcaggatgaggctgatcgacgcggaaaggtctatgacaaatacatgtccagcttcctcttcaacctcaataatgattttgtagtggatgctactcggaaaggaaacaaaattcgatttgcaaatcattcagtgaatcccaactgttatgccaaagtggtcatggtgaatggagaccatcggattgggatctttgccaagagggcaattcaagctggcgaagagctcttctttgattacaggtacagccaagctgatgctctcaagtacgtggggatcgagagggagaccgacgtcctttagccctcccaggccccacggcagcacttatggtagcggcactgtcttggctttcgtgctcacaccactgctgctcgagtctcctgcactgtgtctcccacactgagaaaccccccaacccactccctctgtagtgaggcctctgccatgtccagagggcacaaaactgtctcaatgagaggggagacagaggcagctagggcttggtctcccaggacagagagttacagaaatgggagactgtttctctggcctcagaagaagcgagcacaggctggggtggatgacttatgcgtgatttcgtgtcggctccccaggctgtggcctcaggaatcaacttaggcagttcccaacaagcgctagcctgtaattgtagctttccacatcaagagtccttatgttattgggatgcaggcaaacctctgtggtcctaagacctggagaggacaggctaagtgaagtgtggtccctggagcctacaagtggtctgggttagaggcgagcctggcaggcagcacagactgaactcagaggtagacaggtcaccttactacctcctccctcgtggcagggctcaaactgaaagagtgtgggttctaagtacaggcattcaaggctgggggaaggaaagctacgccatccttccttagccagagagggagaaccagccagatgatagtagttaaactgctaagcttgggcccaggaggctttgagaaagccttctctgtgtactctggagatagatggagaagtgttttcagattcctgggaacagacaccagtgctccagctcctccaaagttctggcttagcagctgcaggcaagcattatgctgctattgaagaagcattaggggtatgcctggcaggtgtgagcatcctggctcgctggatttgtgggtgttttcaggccttccattccccatagaggcaaggcccaatggccagtgttgcttatcgcttcagggtaggtgggcacaggcttggactagagaggagaaagattggtgtaatctgctttcctgtctgtagtgcctgctgtttggaaagggtgagttagaatatgttccaaggttggtgaggggctaaattgcacgcgtttaggctggcaccccgtgtgcagggcacactggcagagggtatctgaagtgggagaagaagcaggtagaccacctgtcccaggctgtggtgccaccctctctggcattcatgcagagcaaagcactttaaccatttcttttaaaaggtctatagattggggtagagtttggcctaaggtctctagggtccctgcctaaatcccactcctgagggagggggaagaagagagggtgggagattctcctccagtcctgtctcatctcctgggagaggcagacgagtgagtttcacacagaagaatttcatgtgaatggggccagcaagagctgccctgtgtccatggtgggtgtgccgggctggctgggaacaaggagcagtatgttgagtagaaagggtgtgggcgggtatagattggcctgggagtgttacagtagggagcaggcttctcccttctttctgggactcagagccccgcttcttcccactccacttgttgtcccatgaaggaagaagtggggttcctcctgacccagctgcctcttacggtttggtatgggacatgcacacacactcacatgctctcactcaccacactggagggcacacacgtaccccgcacccagcaactcctgacagaaagctcctcccacccaaatgggccaggccccagcatgatcctgaaatctgcatccgccgtggtttgtattcattgtgcatatcagggataccctcaagctggactgtgggttccaaattactcatagaggagaaaaccagagaaagatgaagaggaggagttaggtctatttgaaatgccaggggctcgctgtgaggaataggtgaaaaaaaacttttcaccagcctttgagagactagactgaccccacccttccttcagtgagcagaatcactgtggtcagtctcctgtcccagcttcagttcatgaatactcctgttcctccagtttcccatcctttgtccctgctgtcccccacttttaaagatgggtctcaacccctccccaccacgtcatgatggatggggcaaggtggtggggactaggggagcctggtatacatgcggcttcattgccaataaatttcatgcactttaaagtcctgtggcttgtgacctcttaataaagtgttagaatcca

SEQ ID NO: 71 - RNA, 4606 nt, Homo sapiens
  source 1..4606: mol_type = genomic RNA
agcagcgggacccgcggctcgggatggaggctggacacctgttctgctgttgtgtcctgccattctcctgaagaacagaggcacactgtaaaacccaacacttccccttgcattctataagattacagcaagatggaaataccaaatccccctacctccaaatgtatcacttactggaaaagaaaagtgaaatctgaatacatgcgacttcgacaacttaaacggcttcaggcaaatatgggtgcaaaggctttgtatgtggcaaattttgcaaaggttcaagaaaaaacccagatcctcaatgaagaatggaagaagcttcgtgtccaacctgttcagtcaatgaagccttgtaccatagagagcattttcccgggatttgcaagccaacatatgttaatgaggtcactgaacacagttgcattggttcccatcatgtattcctggtcccctctccaacagaactttatggtagaagatgagacggttttgtgcaatattccctacatgggagatgaagtgaaagaagaagatgagacttttattgaggagctgatcaataactatgatgggaaagtccatggtgaagaagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggcaacaaaaagagttccaagaaacagttcccaaatgacatgatcttcagtgcaattgcctcaatgttccctgagaatggtgtcccagatgacatgaaggagaggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcacccttttcatgccacccctaatgtatataaacgcaagaataaagaaatcaagattgaaccagaaccatgtggcacagactgcttccttttgctggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtctttcagtttgcagtcaaagaatcacttatcctgaagctgccaacagatgagctcatgaacccctcacagaagaagaaaagaaagcacagattgtgggctgcacactgcaggaagattcagctgaagaaagataactcttccacacaagtgtacaactaccaaccctgcgaccacccagaccgcccctgtgacagcacctgcccctgcatcatgactcagaatttctgtgagaagttctgccagtgcaacccagactgtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaagcacctgctgctggccccctctgatgtggccggatggggcaccttcataaaggagtctgtgcagaagaacgaattcatttctgaatactgtggtgagctcatctctcaggatgaggctgatcgacgcggaaaggtctatgacaaatacatgtccagcttcctcttcaacctcaataatgattttgtagtggatgctactcggaaaggaaacaaaattcgatttgcaaatcattcagtgaatcccaactgttatgccaaagtggtcatggtgaatggagaccatcggattgggatctttgccaagagggcaattcaagctggcgaagagctcttctttgattacaggtacagccaagctgatgctctcaagtacgtggggatcgagagggagaccgacgtcctttagccctcccaggccccacggcagcacttatggtagcggcactgtcttggctttcgtgctcacaccactgctgctcgagtctcctgcactgtgtctcccacactgagaaaccccccaacccactccctctgtagtgaggcctctgccatgtccagagggcacaaaactgtctcaatgagaggggagacagaggcagctagggcttggtctcccaggacagagagttacagaaatgggagactgtttctctggcctcagaagaagcgagcacaggctggggtggatgacttatgcgtgatttcgtgtcggctccccaggctgtggcctcaggaatcaacttaggcagttcccaacaagcgctagcctgtaattgtagctttccacatcaagagtccttatgttattgggatgcaggcaaacctctgtggtcctaagacctggagaggacaggctaagtgaagtgtggtccctggagcctacaagtggtctgggttagaggcgagcctggcaggcagcacagactgaactcagaggtagacaggtcaccttactacctcctccctcgtggcagggctcaaactgaaagagtgtgggttctaagtacaggcattcaaggctgggggaaggaaagctacgccatccttccttagccagagagggagaaccagccagatgatagtagttaaactgctaagcttgggcccaggaggctttgagaaagccttctctgtgtactctggagatagatggagaagtgttttcagattcctgggaacagacaccagtgctccagctcctccaaagttctggcttagcagctgcaggcaagcattatgctgctattgaagaagcattaggggtatgcctggcaggtgtgagcatcctggctcgctggatttgtgggtgttttcaggccttccattccccatagaggcaaggcccaatggccagtgttgcttatcgcttcagggtaggtgggcacaggcttggactagagaggagaaagattggtgtaatctgctttcctgtctgtagtgcctgctgtttggaaagggtgagttagaatatgttccaaggttggtgaggggctaaattgcacgcgtttaggctggcaccccgtgtgcagggcacactggcagagggtatctgaagtgggagaagaagcaggtagaccacctgtcccaggctgtggtgccaccctctctggcattcatgcagagcaaagcactttaaccatttcttttaaaaggtctatagattggggtagagtttggcctaaggtctctagggtccctgcctaaatcccactcctgagggagggggaagaagagagggtgggagattctcctccagtcctgtctcatctcctgggagaggcagacgagtgagtttcacacagaagaatttcatgtgaatggggccagcaagagctgccctgtgtccatggtgggtgtgccgggctggctgggaacaaggagcagtatgttgagtagaaagggtgtgggcgggtatagattggcctgggagtgttacagtagggagcaggcttctcccttctttctgggactcagagccccgcttcttcccactccacttgttgtcccatgaaggaagaagtggggttcctcctgacccagctgcctcttacggtttggtatgggacatgcacacacactcacatgctctcactcaccacactggagggcacacacgtaccccgcacccagcaactcctgacagaaagctcctcccacccaaatgggccaggccccagcatgatcctgaaatctgcatccgccgtggtttgtattcattgtgcatatcagggataccctcaagctggactgtgggttccaaattactcatagaggagaaaaccagagaaagatgaagaggaggagttaggtctatttgaaatgccaggggctcgctgtgaggaataggtgaaaaaaaacttttcaccagcctttgagagactagactgaccccacccttccttcagtgagcagaatcactgtggtcagtctcctgtcccagcttcagttcatgaatactcctgttcctccagtttcccatcctttgtccctgctgtcccccacttttaaagatgggtctcaacccctccccaccacgtcatgatggatggggcaaggtggtggggactaggggagcctggtatacatgcggcttcattgccaataaatttcatgcactttaaagtcctgtggcttgtgacctcttaataaagtgttagaatcca

SEQ ID NO: 72 - RNA, 2639 nt, Homo sapiens
  source 1..2639: mol_type = genomic RNA
gtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 73 - RNA, 2612 nt, Homo sapiens
  source 1..2612: mol_type = genomic RNA
gtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttcttgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 74 - RNA, 2647 nt, Homo sapiens
  source 1..2647: mol_type = genomic RNA
ggagtagcttcgcctctgacgtttccccacgacgcaccccgaaatccccctgagctccggcggtcgcgggctgccctcgccgcctggtctggctttatgctaagtttgagggaagagtcgagctgctctgctctctattgattgtgtttctggagggcgtcctgttgaattcccacttcattgtgtacatccccttccgttccccccaaaaatctgtgccacagggttactttttgaaagcgggaggaatcgagaagcacgatcttttggaaaacttggtgaacgcctaaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttcttgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaagggtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 75 - RNA, 2654 nt, Homo sapiens
  source 1..2654: mol_type = genomic RNA
gtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatcgtaagtgcaattattcttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 76 - RNA, 2522 nt, Homo sapiens
  source 1..2522: mol_type = genomic RNA
gtttggcgctcggtccggtcgcgtccgacacccggtgggactcagaaggcagtggagccccggcggcggcggcggcggcgcgcgggggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggaggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaatttttgaagccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgttttttgattacagatacagccaggctgatgccctgaagtatgtcggcatcgaaagagaaatggaaatcccttgacatctgctacctcctcccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgtc

SEQ ID NO: 77 - DNA, 40 bp, synthetic construct
  source 1..40: mol_type = other DNA
aaattccacccacgagacccaaaccttctcctgtcagtaa

(19) United States
(12) Patent Application Publication
(10) Pub. No.: US 2024/0041998 A1
(43) Pub. Date: Feb. 8, 2024

(54) METHODS AND COMPOSITIONS FOR SENSITIZING PRC2 MUTANT TUMORS TO IMMUNE CHECKPOINT BLOCKADE THERAPY

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Ping CHI, New York, NY (US); Yu CHEN, New York, NY (US); Juan YAN, New York, NY (US)

(21) Appl. No.: 18/350,454

(22) Filed: Jul. 11, 2023

Related U.S. Application Data
(60) Provisional application No. 63/388,102, filed on Jul. 11, 2022.

(51) Int. Cl. A61K 39/00; A61K 39/395; A61P 35/00

(52) U.S. Cl. CPC A61K 39/0011; A61K 39/3955; A61P 35/00; A61K 2039/5252; A61K 2039/585

(57) ABSTRACT

The present disclosure relates generally to methods for sensitizing PRC2 mutant tumors to immune checkpoint blockade therapy in a subject in need thereof comprising administering to the subject an effective amount of inactivated modified vaccinia virus Ankara (MVA) or MVA ΔE3L.

### CROSS-REFERENCE TO RELATED APPLICATIONS

This application claims the benefit of and priority to U.S. Provisional Patent Application No. 63/388,102 filed Jul. 11, 2022, the entire contents of which is incorporated herein by reference.

### SEQUENCE LISTING

The instant application contains a Sequence Listing which has been submitted electronically in XML file format and is hereby incorporated by reference in its entirety. Said XML copy, created on Sep. 13, 2023, is named 115872-2823_SL.xml and is 115,004 bytes in size.

### TECHNICAL FIELD

The present technology relates generally to methods for sensitizing PRC2 mutant tumors to immune checkpoint blockade therapy in a subject in need thereof comprising administering to the subject an effective amount of inactivated modified vaccinia virus Ankara (MVA) or MVA ΔE3L.

### BACKGROUND

The following description of the background of the present technology is provided simply as an aid in understanding the present technology and is not admitted to describe or constitute prior art to the present technology.

The PRC2 complex consisting of core components of EZH1/2, EED, and SUZ12, establishes and maintains H3K27me2/3 in the genome and regulates chromatin structure, transcription, cellular stemness, and differentiation [1]. PRC2 is a context-dependent tumor suppressor whose core components are frequently inactivated genetically or epigenetically in various cancer types, including malignant peripheral nerve sheath tumor (MPNST) [2, 3], melanoma [2], myeloid disorders [4, 5], T-cell acute lymphocytic leukemia (ALL) [6], and early T-cell precursor ALL [7], pediatric gliomas [8-11], invasive breast cancer [12], and others. Among all cancer types, high-grade MPNST, a group of aggressive soft tissue sarcomas with no effective therapies, has the highest prevalence of complete loss of PRC2 function through biallelic inactivation of the PRC2 core components, EED or SUZ12 [2, 3, 13-15].

Therapeutically targeting PRC2-inactivation in cancer remains a challenge and requires relevant context specific cancer models for therapeutic discovery and development.

### SUMMARY OF THE PRESENT TECHNOLOGY

In one aspect, the present disclosure provides a method for selecting a cancer patient for treatment with an immune checkpoint inhibitor comprising (a) detecting the presence of at least one mutation that results in reduced expression or activity of Polycomb Repressive Complex 2 (PRC2) in a biological sample obtained from the cancer patient; and (b) administering to the cancer patient an effective amount of inactivated modified vaccinia virus Ankara (MVA) or MVA ΔE3L and an effective amount of an immune checkpoint inhibitor. The at least one mutation may comprise a mutation in SUZ12, EED, and/or EZH1/2. In some embodiments, the at least one mutation is a nonsense mutation, a missense mutation, a deletion, an inversion or a frameshift mutation. Additionally or alternatively, in some embodiments, the at least one mutation is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

In one aspect, the present disclosure provides a method for treating cancer in a patient in need thereof comprising administering to the patient an effective amount of inactivated modified vaccinia virus Ankara (MVA) or MVA ΔE3L and an effective amount of an immune checkpoint inhibitor, wherein mRNA and/or polypeptide expression and/or activity levels of PRC2 in a biological sample obtained from the patient are reduced compared to a control sample obtained from a healthy subject or a predetermined threshold. In certain embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). In other embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any and all embodiments of the methods disclosed herein, the biological sample obtained from the cancer patient comprises biopsied tumor tissue, whole blood, plasma, or serum.

In another aspect, the present disclosure provides a method for sensitizing PRC2 mutant tumors to treatment with an immune checkpoint inhibitor in a patient in need thereof comprising administering to the patient an effective amount of inactivated modified vaccinia virus Ankara (MVA) or MVA ΔE3L separately, sequentially or simultaneously with the immune checkpoint inhibitor.

In any of the preceding embodiments of the methods disclosed herein, inactivation of MVA or MVA ΔE3L occurs via heat-induced inactivation or UV radiation-induced inactivation. Examples of immune checkpoint inhibitors include, but are not limited to one or more of a PD-1/PD-L1 inhibitor, a CTLA-4 inhibitor, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, and BMS-986189.

In any and all embodiments of the methods disclosed herein, the patient suffers from or is diagnosed with malignant peripheral nerve sheath tumor (MPNST), melanoma, a myeloid disorder, T-cell acute lymphocytic leukemia (ALL), early T-cell precursor ALL, pediatric glioma, or invasive breast cancer.

In any and all embodiments of the methods disclosed herein, the immune checkpoint inhibitor and/or inactivated MVA or MVA ΔE3L is administered orally, intranasally, parenterally, intravenously, intramuscularly, intraperitoneally, subcutaneously, rectally, intrathecally, intratumorally or topically.

### BRIEF DESCRIPTION OF THE DRAWINGS

FIGS. 1A-1E: PRC2 loss is associated with deficiency of broad subclasses of tumor immune infiltrates in MPNST. FIG. 1A: Differential gene sets between PRC2-wt and PRC2-loss human MPNST tumors at transcriptome level by GSEA analysis of RNA-seq. Blank dash line: top 100 gene sets in rank. FIGS. 1B-1C: Representative immunohistochemistry (IHC) (FIG. 1B) and quantification (FIG. 1C) of H3K27me3 and CD45 in PRC2-loss (n=69) and PRC2-wt (n=39) MPNST tumors. Scale bar: 100 μm. FIGS. 1D-1E: Representative IHC (FIG. 1D) and quantification (FIG. 1E) of H3K27me3 and immune cell markers in PRC2-loss (n>27) and PRC2-wt (n>14) MPNST tumor tissue microarray. Scale bar: 100 μm. *P<0.05, **P<0.01, ***P<0.001, ****P<0.0001 by unpaired two-tailed t test in FIG. 1C and FIG. 1E. All error bars: mean±SEM.

FIGS. 2A-2D: Antigen presentation and IFNγ pathway are diminished in PRC2-loss MPNST. FIG. 2A: GSEA of human MPNST transcriptomes points to antigen presentation and IFNγ pathway defects in PRC2-loss compared to PRC2-wt tumors. FIG. 2B: Heatmap of IFNγ signaling and downstream genes in human MPNSTs. FIGS. 2C-2D: Representative IHC (FIG. 2C) and quantification (FIG. 2D) of MHC I, MEW II and B2M in PRC2-loss (n>27) and PRC2-wt (n>14) human MPNST tissue microarray. Scale bar: 100 μm. ****P<0.0001 by Chi-square test for contingency in FIG. 2D.

FIGS. 3A-3K: PRC2 loss reprograms the chromatin landscape and suppresses a subset of IFNγ-responsive genes. FIG. 3A: Immunoblots and representative IHC of indicated proteins in PRC2-isogenic human MPNST cells (left) and orthotopically (sciatic nerve pocket) transplanted MPNST tumors (right). Scale bar: 50 μm. FIG. 3B: Immunoblots of indicated proteins from different fractions of PRC2-isogenic M3 cells. Lamin B1, HIRA and GAPDH are controls. FIG. 3C: A volcano plot of chromatin accessibility changes by ATAC-seq. Red-dots represent significantly changed ATAC peak (FDRq<0.1, fold-change ≥1.5). FIG. 3D: Density plot of histone modifications by ChIP-seq, centered on significantly increased (left) and decreased ATAC-peaks (right) by SUZ12 knockout in M3 cells. Promoter: transcriptional start site (TSS)±2 kb; Non-promoters: rest of the genome other than promoter, including distal regulatory enhancer and intergenic regions. FIG. 3E: Distribution of differential H3K27ac peaks across different genomic regions in PRC2-isogenic M3 cells (FDR q<0.05, fold-change ≥2); promoter (TSS±2 kb), distal regulatory (−50 kb from TSS to transcriptional end site [TES]+5 kb) and intergenic (non-promoter, non-distal regulatory) regions, as well as at super enhancers (SE). FIG. 3F: Violin plots of mRNA baseline expression of genes associated with PRC2-loss induced significantly increased (SigUP) and decreased (SigDN) H3K27ac at their respective loci in M3 sgCon cells. FIG. 3G: The correlation of significant transcriptome and H3K27ac changes at promoter (TSS±2 kb) and non-promoter regions (FDR q<0.05, fold-change ≥2). Blue and grey dots represent peaks mapped to genes with (DiffExpGene) and without (nonDiffExpGene) significant transcriptome changes, respectively. FIG. 3H: HOMER motif analysis of PRC2 loss-associated significantly decreased and increased H3K27ac peaks in PRC2-isogenic M3 cells. e=exponents of 10. FIGS. 3I-3K: ChIP-seq and ATAC-seq profiles at the loci of indicated IFNγ-responsive genes and others in PRC2-isogenic M3 cells. Pink: H3K27ac change; Yellow: H3K27me3 change. RPL27: reference gene.

FIGS. 4A-4F: PRC2 loss dampens the IFNγ response in tumor cells through decreasing chromatin accessibility. FIG. 4A: Principal component analysis of chromatin accessibility by ATCA-seq in PRC2-wt (sgCon) and PRC2-loss (sgSUZ12) human MPNST cells with or without IFNγ stimulation. FIG. 4B: K-means clustering analysis of the chromatin accessibility changes in PRC2-isogenic M3 cells with or without IFNγ stimulation. Cells were treated with or without 10 ng/ml IFNγ for 24 hours followed by ATAT-seq. Homer de novo motif analysis enriches IRF only in cluster 2, 6 and 7. e=exponents of 10. FIGS. 4C-4D: Comparison of Go analysis between cluster 7 and cluster 2 related to FIG. 4B. FIGS. 4E-4F: IFNγ dose-dependent mRNA expression changes of IFNγ-responsive genes without (FIG. 4E) or with (FIG. 4F) PRC2 loss-associated chromatin accessibility changes by qRT-PCR. n=3. All error bars: mean±SEM.

FIGS. 5A-5G: PRC2 loss suppresses tumor immune infiltration in a murine MPNST syngeneic transplant model. FIG. 5A: Immunoblots of indicated proteins in PRC2-loss (sgEed) and PRC2-wt (sgCon) isogenic murine MPNST SKP605 cells. FIG. 5B: A schematic of the experimental and analysis plan of an orthotopic and syngeneic transplantable murine MPNST SKP605 model. FIG. 5C: Tumor volumes of PRC2-isogenic (sgEed vs. sgCon) murine SKP605 tumors on day 26 post sciatic nerve pocket implantation. n=10 tumors bilaterally grafted in C57BL/6J mice for each cohort. FIG. 5D: Immunoblots of indicated proteins of PRC2-wt and PRC2-loss murine MPNST tumors in FIG. 5C. FIGS. 5E-5F: Percentage of total immune cells CD45⁺ (FIG. 5E) and subpopulations in total live cells (FIG. 5F) in PRC2-isogenic murine SKP605 tumors in FIG. 5C, n=5 tumors for each cohort. FIG. 5G: The percentage of IFNγ⁺CD4⁺ or IFNγ⁺CD8⁺ T cells of total live cells in PRC2-isogenic SKP605 tumors. n=5 tumors for each group. *P<0.05, **P<0.01, ****P<0.0001 by unpaired two-tailed t test in FIG. 5C and FIGS. 5E-5G. All error bars: mean±SEM.

FIGS. 6A-6I: PRC2 loss leads to an antigen presentation defect and recapitulates the immune evasion phenotype in a murine mammary tumor syngeneic transplant model. FIG. 6A: A schematic of murine AT3 mammary tumor experimental plans in vivo. FIG. 6B: Growth curves of PRC2-isogeneic (sgCon vs. sgEed) AT3 syngeneic transplant tumors overtime. n=10 tumors in each group. FIG. 6C: Immunoblots of H3K27me3 in pre-graft AT3 cells and explanted AT3 tumor grafts. FIG. 6D: GSEA of transcriptomes derived from PRC2-isogenic AT3 (sgCon vs. sgEed) tumors in FIG. 6B. sgCon: n=4 tumors; sgEed: n=8 tumors from 4 sgEed-1 and 4 sgEed-2 tumors. FIG. 6E: Heatmap of expression changes for genes related to antigen presentation, IFNγ signaling response, chemokines and WNT signaling from RNA-seq of AT3 tumors. FIGS. 6F-6G: Percentage of CD45⁺ total immune cells (FIG. 6F) and subpopulations (FIG. 6G) in total live cells in PRC2-isogenic AT3 syngeneic graft tumors. n=5 tumors for each group. FIG. 6H: The percentage of IFNγ⁺ cells in total CD4⁺ and CD8⁺ T cells in PRC2-isogenic AT3 tumors. n=10 tumors for each group. FIG. 6I: Tetramer staining of OVA-specific CD8⁺ T cells in TdLNs 14 days after transplanting PRC2-isogenic AT3 cells in C57BL/6J mice. TdLNs for OVA⁺ tumors: n=4 for each group, TdLN for OVA⁻ sgCon tumors: n=3. *P<0.05, **P<0.01, ***P<0.001, ****P<0.0001 by one-way ANOVA in FIGS. 6B and 6F-611, and by unpaired two-tailed t test in I. All error bars: mean±SEM.

FIGS. 7A-7I: PRC2-loss tumors confer primary resistance to immune checkpoint blockade (ICB) therapies. FIG. 7A: A schematic of treatment plan of PRC2-loss AT3 tumors with ICB. FIG. 7B: Tumor growth curves of PRC2-isogenic AT3 tumors with ICB treatment and controls overtime. n=10 tumors for each group. FIG. 7C: Percentage of CD45⁺ tumor immune cells in all live cells in PRC2-isogneic AT3 tumors (left) or spleens (right). n=5 for each cohort. FIG. 7D: Representative examples of CD45⁺ cells (gated CD45⁺ cells in all live cells) by FACS for FIG. 7C. FIG. 7E: Percentage of T cells in PRC2-isogenic AT3 tumors treated with ICB and controls. n=5 for each cohort. FIG. 7F: Representative examples of T cells (gated TCRβ⁺ in CD45⁺ cells) by FACS for FIG. 7E. FIG. 7G: Percentage of IFNγ⁺CD4⁺ and CD8⁺ T cells in all live cells in tumors. n=5 for each cohort. FIG. 7H: Tumor growth curves of AT3 sgCon tumors with indicated treatment overtime. n=6-10 tumors per group. FIG. 7I: mRNA expression level change of gene Ifng in tumors from FIG. 711 by qRT-PCR. n=4 tumors per group. *P<0.05, **P<0.01, ***P<0.001, ****P<0.0001 by unpaired two-tailed t test in FIGS. 7B-7C, 7E and 7G-7I. All error bars: mean±SEM.

FIGS. 8A-8L: Intratumoral injection of heat-iMVA sensitizes PRC2-loss tumors to anti-PD1 and anti-CTLA4 therapy in murine cancer models. FIGS. 8A-8B: mRNA expression level change of type I IFN (FIG. 8A) and IFNγ-responsive genes (FIG. 8B) 24 hours post heat-iMVA infection in vitro by qRT-PCR. MOI=10. FIG. 8C: A schematic of treatment plan of heat-iMVA and ICBs in PRC2-loss AT3 or SKP605 (sgEed) tumors. FIGS. 8D-8E: Tumor growth curves (FIG. 8D) and Kaplan-Meier survival curves (FIG. 8E) overtime in mice with AT3 sgEed tumors under indicated treatment. n=10 per group. FIG. 8F: Percentage (upper) and representative FACS profiles (lower) of tumor infiltrating CD45⁺ immune cells in AT3 (sgEed) tumors under indicated treatments. n=5 for each cohort. FIG. 8G: Percentage of CD4⁺ and CD8⁺ T cells in all live cells and the relative Ki67⁺ subpopulation in AT3 (sgEed) tumors under indicated treatments. FIGS. 8H-8I: Percentage of FoxP3⁺ Treg cells in CD4⁺ T cells (FIG. 811) and GzmB⁺ cells in CD8⁺ T cells (FIG. 8I) in AT3 (sgEed) tumors under indicated treatment. n=5 for each cohort. FIGS. 8J-8K: Tumor growth curves for each tumor (FIG. 8J) and Kaplan-Meier survival curves (FIG. 8K) overtime in mice with SKP605 (sgEed) tumors under indicated treatment. SKP605 sgEed cells were subcutaneously grafted on the right flank of C57BL/6J mice. n=8-10 tumors per group. FIG. 8L: Tumor growth curves for each tumor subcutaneously grafted on the left flank of C57BL/6J mice. n=7-10 tumors per group. *P<0.05, **P<0.01, ***P<0.001, ****P<0.0001 by unpaired two-tailed t test in FIGS. 8D, 8G-8I between two groups; by Log-rank (Mantel-Cox) test in FIG. 8E and FIG. 8K; by one-way ANOVA in FIGS. 8G-8I among three groups. All error bars: mean±SEM.

FIGS. 9A-9F: PRC2-loss tumors have an immune-desert microenvironment in human cancer, Related to FIG. 1. FIG. 9A: A schematic of human MPNST tumor tissue sample handling and processing. FIG. 9B: Principal component analysis (PCA) of all (1′ and 2n d batch) tumor transcriptomes by RNA-seq. FIG. 9C: Hierarchical clustering of the most differentially regulated genes (FDR q<0.05, and fold-change>8) between PRC2-loss and PRC2-wt MPNST tumors in RNA-seq. FIG. 9D: Heatmap of WNT signaling pathway genes in human MPNSTs by RNA-seq. FIG. 9E: Tukey's box and whiskers plots of mRNA expression levels of immune maker genes in human MPNST by RNA-seq. TPM: transcripts per million. ***P<0.001, ****P<0.0001 by unpaired two-tailed t test. FIG. 9F: Representative IHC and quantification of the immune cell population in human PRC2-loss tumors in MSK-IMPACT. Scale bar: 50 μm. All error bars: mean±SEM.

FIGS. 10A-10J: H3K27ac change correlates the chromatin accessibility and transcription change after PRC2 loss in cancer cells, Related to FIG. 3. FIG. 10A: Genome-wide distribution of significantly decreased (left) and increased (right) chromatin accessibility sites. promoter (TSS±2 kb), distal regulatory (−50 kb from TSS to transcriptional end site [TES]+5 kb) and intergenic (non-promoter, non-distal regulatory) regions. FIG. 10B: Pie chart of the overlap of the significant ATAC-seq peaks with H3K27me3 enrichment, comparing PRC2-isogenic M3 cells. FIG. 10C: ChIP-seq and ATAC-seq profiles at the loci of selective genes with increased (left) and decreased (right) H3K27ac enrichment and chromatin accessibility in PRC2-isogenic M3 cells. FIGS. 10D-10E: Density plot of various histone modifications by CUT&RUN, centered by significantly changed ATAC peaks (FIG. 10D) and H3K27me3 peaks (FIG. 10E) in PRC2-isogenic M3 cells. FIG. 10F: IGV tracks of indicated histone modifications at the selective loci in PRC2-isogenic M3 cells by CUT&RUN. Yellow: H3K27me3 change; Pink: H3K27ac change; Grey: background region. FIG. 10G: Pie chart of the overlap of the significant H3K27ac peaks with H3K27me3 enrichment in PRC2-isogenic M3 cells. FIG. 10H: The distance of changed H3K27ac peaks to the nearest H3K27me3 and H3K36me3 peaks. FIG. 10I: mRNA expression level change of gene CCL2 72 hours after indicated inhibitor treatment in M3 cells. FIG. 10J: A cartoon model about H3K27ac redistribution or spreading after global H3K27me3 loss. ****P<0.0001 by unpaired two-tailed t test for two groups while by one-way ANOVA among 3 groups in I. All error bars: mean±SEM.

FIGS. 11A-11F: IFNγ response is diminished by PRC2 loss in human MPNST cells, Related to FIG. 4. FIG. 11A: Immunoblots of indicated proteins in PRC2-isogenic M3 cells stimulated with IFNγ (10 ng/ml) for 24 hours. FIG. 11B: mRNA expression levels of gene SUZ12 and EED by qRT-PCR in PRC2-isogenic M3 cells after stimulation with IFNγ (10 ng/ml) for 24 hours. n=4. FIG. 11C: IFNγ dose-dependent mRNA expression change of IFNγ-responsive genes by qRT-PCR. n=3. FIGS. 11D-11E: Target validation of engineered sNF96.2 cells by western blot (FIG. 11D) and flow cytometry (FIG. 11E). Dox: doxycycline. 1 μg/ml for 2 weeks. FIG. 11F: mRNA expression level change of genes in PRC2-isogenic sNF96.2 cells in FIG. 11D with or without IFNγ (10 ng/ml) for 24 hours by qRT-PCR. n=3. *P<0.05, **P<0.01, ***P<0.001, ****P<0.0001 by unpaired two-tailed t test in FIG. 11B and FIG. 11F. All error bars: mean±SEM.

FIGS. 12A-12I: PRC2 loss promotes immune evasion in a murine MPNST model amenable for orthotopic and syngeneic transplant, Related to FIG. 5. FIG. 12A: A schematic of the development of a murine MPNST SKP605 model amenable for orthotopic (sciatic nerve) and syngeneic transplant in C57BL/6J mice. FIG. 12B: Representative histology and IHC of indicated proteins in orthotopically transplanted murine MPNST tumors derived from step 5 in FIG. 12A. Scale bar: 50 μm. FIG. 12C: Left: A schematic of Eed single guide RNA location and PCR primer location for amplicon-seq; Right, percent of mutations relative to amplicon position at sgEed targeted region by CRISPR-sequencing in SKP605 cells. Figure discloses SEQ ID NO: 77. FIG. 12D: Images (left) and tumor sizes (right) of orthotopically and syngeneic transplant murine MPNST PRC2-isogenic (sgCon vs. sgEed) SKP605 on day 26 post-transplant. Diameter=(length+width+height)/3. n=10 tumors per group. FIG. 12E: mRNA expression level change of indicated genes by qRT-PCR in PRC2-isogenic SKP605 tumors in FIG. 12D. n=5 tumors per group. FIGS. 12F-12G: Percentage of CD45⁺ total immune cells (FIG. 12F) and the subpopulations (FIG. 12G) in all live cells in the spleens of mice with PRC2-isogenic tumors. n=5 spleens for each group. FIG. 12H: The percentage of functional T cells in spleen. n=5 spleens per group. FIG. 12I: Representative IHC of immune cell populations in PRC2-isogenic SKP605 tumors in FIG. 12D. Scale bar: 100 μm. *P<0.05, **P<0.01, ***P<0.001, ****P<0.0001 by unpaired two-tailed t test in FIGS. 12D-1211. All error bars: mean±SEM.

FIG. 13: Gating strategies of multi-color flow cytometry for immune profile, Related to FIG. 5. Representative example of CD45, B220, F4/80, MHCII, CD11c, CD11b, TCRβ, CD4, CD8 staining to show gating skills (gated for total immune cells, dendritic cells, T cells, macrophages, and B cells) for different immune subpopulations by FACS.

FIGS. 14A-14M: PRC2 loss promotes immune evasion in a murine breast cancer model, Related to FIG. 6. FIG. 14A, FIG. 14K: Percent of mutations relative to amplicon position at sgEed targeted region by CRISPR-sequencing in AT3 (FIG. 14A) and AT3-USA cells (FIG. 14K). FIG. 14B: Clonogenic assays of PRC2-isogenic AT3 cells in vitro. FIG. 14C: Tumor growth curves of PRC2-isogenic (sgCon vs. sgSuz12) AT3 tumors after mammary fat pad graft in C57BL/6J mice. n=10 tumors for each group. FIG. 14D: Immunoblots of indicated proteins in representative AT3 tumors in (FIG. 14C). FIG. 14E: Representative IHC of CD45⁺ immune cells in PRC2-isogenic AT3 tumors. Scale bar: 50 μm. FIGS. 14F-1411: Percentage of CD45⁺ total immune cells (FIG. 14F), its subpopulations (FIG. 14G) and functional T cells (FIG. 14H) in spleens of mice with PRC2-isogenic AT3 tumors. n=5 spleens per group. FIGS. 14I-14J: Percentage of GzmB⁺ (FIG. 14I) and TNFα⁺ (FIG. 14J) cells in CD4⁺ or CD8⁺ T cells in PRC2-isogenic AT3 tumors (upper) and spleens (lower). n=10 tumors per group; n=3-4 spleens per group. FIG. 14L: Representative immunoblots of indicated proteins in pre-graft AT3 cells and explanted tumor grafts. FIG. 14M: Absolute cell numbers of MHCI-OVA tetramer⁺ CD8⁺ T cells in TdLNs related to FIG. 6I. *P<0.05, **P<0.01, ****P<0.0001 by unpaired two-tailed t test in FIG. 14C and FIG. 14M, by one-way ANOVA in FIGS. 14F-14J. All error bars: mean±SEM.

FIGS. 15A-15E: PRC2 loss confers primary resistance to ICB in murine syngeneic transplant mammary tumor models, Related to FIG. 7. FIG. 15A: Representative IHC of CD45⁺ immune cells in PRC2-isogenic AT3 tumors related to FIG. 7B. Scale bar: 50 μm. FIG. 15B: Percentage of T cells in the spleens of mice related to FIG. 7B. n=5 for each cohort. FIG. 15C: Percentage of subpopulations of immune cells in PRC2-isogenic AT3 tumors (left) and spleens (right) of mice related to FIG. 7B. n=5 for each cohort. FIG. 15D: The immune cell population change in PBMC after depletion antibody treatment for 19 days related to FIG. 711. FIG. 15E: mRNA expression level change of genes in tumors related to FIG. 711 by qRT-PCR. *P<0.05, **P<0.01, ****P<0.0001 by unpaired two-tailed t test in FIGS. 15B-15C and 15D-15E compared to ICBs group. All error bars: mean±SEM.

FIGS. 16A-16F: Intratumoral injection of heat-iMVA combined with anti-PD1 and anti-CTLA4 inhibits the tumor growth of PRC2-loss tumors, Related to FIG. 8. FIG. 16A: Tumor growth curves of individual AT3 sgEed tumors under indicated treatment. n=10 tumors per group. FIGS. 16B-16E: Percentage of live cells in all cells (FIG. 16B); percentage of subpopulations of immune cells (FIGS. 16C-16E) in AT3 (sgEed) tumors under indicated treatment. n=5 tumors for each cohort. FIG. 16F: Tumor growth curves of SKP605 tumors under indicated treatment. n=6-8 tumors per group. *P<0.05, **P<0.01, ***P<0.001 by unpaired two-tailed t test in FIGS. 16A and 16C-16F between two groups; by one-way ANOVA in FIGS. 16C-16F among three groups. All error bars: mean±SEM.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984)A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

The present disclosure demonstrates that tumor-intrinsic PRC2-loss drives immune evasion through epigenetic reprogramming, and consequently, deficiency in antigen presentation, chemokine production, and IFNγ signaling, and primary resistance to ICB. To overcome the “cold” TME in PRC2-loss cancer, intratumoral delivery of immunogenic MVA demonstrated that the inactivated MVA can enhance anti-tumor immunity, alter the immune-desert TME, and sensitizes the PRC2-loss tumors to ICB therapy. These studies demonstrate that genetic inactivation of PRC2 can be used as a novel biomarker for resistance to ICB therapy in selective cancers and that therapeutic viruses that can enhance anti-tumor immunity to overcome the “cold” TME in PRC2-loss tumors.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3 SR), including multiplex versions or combinations thereof. Copies of a particular nucleic acid sequence generated in vitro in an amplification reaction are called “amplicons” or “amplification products.”

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression. Specifically, a gene refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

“Immune checkpoint inhibitor(s)” as used herein refers to molecules that completely or partially reduce, inhibit, interfere with or modulate the activity of one or more checkpoint proteins. Checkpoint proteins regulate T-cell activation or function. Checkpoint proteins include, but are not limited to CTLA-4 and its ligands CD80 and CD86; PD-1 and its ligands PDL1 and PDL2; LAGS, B7-H3, B7-H4, TIM3, ICOS, and BTLA (Pardoll et al. Nature Reviews Cancer 12: 252-264 (2012)).

As used herein, the terms “individual”, “patient”, or “subject” can be an individual organism, a vertebrate, a mammal, or a human. In some embodiments, the individual, patient or subject is a human.

As used herein, the term “library” refers to a collection of nucleic acid sequences, e.g., a collection of nucleic acids derived from whole genomic, subgenomic fragments, cDNA, cDNA fragments, RNA, RNA fragments, ctDNA, cfDNA, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

As used herein, a “mutation” of a gene or gene product (e.g., a marker gene or gene product) refers to the presence of an alteration or alterations within the gene or gene product, which affects the quantity or activity of the gene or gene product, as compared to the normal or wild-type gene. The genetic alteration can result in changes in the quantity, structure, and/or activity of the gene or gene product in a cancer tissue or cancer cell, as compared to its quantity, structure, and/or activity, in a normal or healthy tissue or cell (e.g., a control). For example, a mutation which is associated with cancer, or predictive of responsiveness to immune checkpoint blockade (ICB) therapy, can have an altered nucleotide sequence, amino acid sequence, chromosomal translocation, intra-chromosomal inversion, copy number, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene.

As used herein, “MVA” means “modified vaccinia Ankara” and refers to a highly attenuated strain of vaccinia derived from the Ankara strain and developed for use as a vaccine and vaccine adjuvant. The original MVA was isolated from the wild-type Ankara strain by successive passage through chicken embryonic cells, Treated thus, it lost about 15% of the genome of wild-type vaccinia including its ability to replicate efficiently in primate (including human) cells. (Mayr et al., Zentralbl Bakteriol B 167, 375-390 (1978)). The smallpox vaccination strain MVA: marker, genetic structure, experience gained with the parenteral vaccination and behavior in organisms with a debilitated defense mechanism. MVA. is considered an appropriate candidate for development as a recombinant vector for gene or vaccination delivery against infectious diseases or tumors. (5S)(Verheust et al., Vaccine 30(16), 2623-2632 (2012)). MVA has a genome of 178 kb in length and a sequence first disclosed in (59)(Antoine et al., Viral. 244(2): 365-396 (1998)). Sequences are also disclosed in Genbank: U94848.1. Clinical grade MVA is commercially and publicly available from Bavarian Nordic A S Kvistgaard, Denmark. Additionally, MVA is available from ATCC, Rockville, MD and from CMCN (Institut Pasteur Collection Nationale des Microorganismes) Paris, France.

“MVA ΔE3L” means a deletion mutant of MVA which lacks a functional E3L gene and is infective but non replicative and it is further impaired in its ability to evade the host's immune system. It has been used as a vaccine vector (by others) to transfer tumor or viral antigens. This mutant MVA E3L knockout and its preparation have been described for example in U.S. Pat. No. 7,049,145.

“Heat-inactivated” with particular reference to vaccinia viruses refers to a virus which has been further treated by exposure to heat under conditions that do not destroy its immunogenicity or its ability to enter target cells (tumor cells) but remove residual replication ability of the virus as well as factors that inhibit the host's immune response (for example, such factors as inhibit the induction of IFN Type I in infected cells). An example of such conditions is exposure to a temperature within the range of about 50 to about 60° C. for a period of time of about an hour. Other times and temperatures can be determined with routine experimentation and IFN Type I induction in infected cDC's can be compared to the Heat-inactivated virus used in experiments described herein and should be higher than that of vaccinia virus.

“UV-inactivated” with particular reference to vaccinia viruses refers to a virus which has been inactivated by exposure to UV under conditions that do not destroy its immunogenicity or its ability to enter target cells (tumor cells) but remove residual replication ability of the virus. An example of such conditions, which can be useful in the present methods, is exposure to UV using for example a 365 nm UV bulb for a period of about 30 min to about 1 hour (Tsung et al. J Virol 70, 165-171 (1996); Drillien, R. et al. J Gen Virol 85: 2167-2175 (2004)).

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, “prevention” or “preventing” of a disorder or condition refers to a compound that, in a statistical sample, reduces the occurrence of the disorder or condition in the treated sample relative to an untreated control sample, or delays the onset of one or more symptoms of the disorder or condition relative to the untreated control sample.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid.

Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment or prevention of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Methods for Detecting Polynucleotides Associated with Responsiveness to DNMT1 Inhibitors

Polynucleotides associated with responsiveness to immune checkpoint blockade (ICB) therapy may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

Nucleic Acid Amplification and/or Detection

Polynucleotides associated with responsiveness to ICB therapy can be detected by the use of nucleic acid amplification techniques that are well known in the art. The starting material may be genomic DNA, cDNA, RNA, ctDNA, cfDNA, or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281: 26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding a polypeptide selected from among SUZ12, EED, and/or EZH1/2. Exemplary nucleic acid sequences of the human orthologs of these genes are provided below:

NM_015355.4 Homo sapiens SUZ12 polycomb repressive complex 2 subunit
(SUZ12), transcript variant 1, mRNA
(SEQ ID NO: 63)
GGAGCGAGGCCAGGGTAGGGTGAGCGGCCTCCGAAGCGGAGCGGGGCTCTGAGGAGACACTTTTTTTTTC
CTCCCTCCTTCCCTCCTCTCCTCCTCCCTTCCCTTCCCCTCTCCTCCCCTCTCTCCTCCTTCCCCCCTCG
GTCCGCCGGAGCCTGCTGGGGCGAGCGGTTGGTATTGCAGGCGCTTGCTCTCCGGGGCCGCCCGGCGGGT
AGCTGGCGGGGGGAGGAGGCAGGAACCGCGATGGCGCCTCAGAAGCACGGCGGTGGGGGAGGGGGCGGCT
CGGGGCCCAGCGCGGGGTCCGGGGGAGGCGGCTTCGGGGGTTCGGCGGCGGTGGCGGCGGCGACGGCTTC
GGGCGGCAAATCCGGCGGCGGGAGCTGTGGAGGGGGTGGCAGTTACTCGGCCTCCTCCTCCTCCTCCGCG
GCGGCAGCGGCGGGGGCTGCGGTGTTACCGGTGAAGAAGCCGAAAATGGAGCACGTCCAGGCTGACCACG
AGCTTTTCCTCCAGGCCTTTGAGAAGCCAACACAGATCTATAGATTTCTTCGAACTCGGAATCTCATAGC
ACCAATATTTTTGCACAGAACTCTTACTTACATGTCTCATCGAAACTCCAGAACAAACATCAAAAGGAAA
ACATTTAAAGTTGATGATATGTTATCAAAAGTAGAGAAAATGAAAGGAGAGCAAGAATCTCATAGCTTGT
CAGCTCATTTGCAGCTTACGTTTACTGGTTTCTTCCACAAAAATGATAAGCCATCACCAAACTCAGAAAA
TGAACAAAATTCTGTTACCCTGGAAGTCCTGCTTGTGAAAGTTTGCCACAAAAAAAGAAAGGATGTAAGT
TGTCCAATAAGGCAAGTTCCCACAGGTAAAAAGCAGGTGCCTTTGAATCCTGACCTCAATCAAACAAAAC
CCGGAAATTTCCCGTCCCTTGCAGTTTCCAGTAATGAATTTGAACCTAGTAACAGCCATATGGTGAAGTC
TTACTCGTTGCTATTTAGAGTGACTCGTCCAGGAAGAAGAGAGTTTAATGGAATGATTAATGGAGAAACC
AATGAAAATATTGATGTCAATGAAGAGCTTCCAGCCAGAAGAAAACGAAATCGTGAGGATGGGGAAAAGA
CATTTGTTGCACAAATGACAGTATTTGATAAAAACAGGCGCTTACAGCTTTTAGATGGGGAATATGAAGT
AGCCATGCAGGAAATGGAAGAATGTCCAATAAGCAAGAAAAGAGCAACATGGGAGACTATTCTTGATGGG
AAGAGGCTGCCTCCATTCGAAACATTTTCTCAGGGACCTACGTTGCAGTTCACTCTTCGTTGGACAGGAG
AGACCAATGATAAATCTACGGCTCCTATTGCCAAACCTCTTGCCACTAGAAATTCAGAGAGTCTCCATCA
GGAAAACAAGCCTGGTTCAGTTAAACCTACTCAAACTATTGCTGTTAAAGAATCATTGACTACAGATCTA
CAAACAAGAAAAGAAAAGGATACTCCAAATGAAAACCGACAAAAATTAAGAATATTTTATCAGTTTCTCT
ATAACAACAATACAAGGCAACAAACTGAAGCAAGAGATGACCTGCATTGCCCTTGGTGTACTCTGAACTG
CCGCAAACTTTATAGTTTACTCAAGCATCTTAAACTCTGCCATAGCAGATTTATCTTCAACTATGTTTAT
CATCCAAAAGGTGCTAGGATAGATGTTTCTATCAATGAGTGTTATGATGGCTCCTATGCAGGAAATCCTC
AGGATATTCATCGCCAACCTGGATTTGCTTTTAGTCGCAACGGACCAGTTAAGAGAACACCTATCACACA
TATTCTTGTGTGCAGGCCAAAACGAACAAAAGCAAGCATGTCTGAATTTCTTGAATCTGAAGATGGGGAA
GTAGAACAGCAAAGAACATATAGTAGTGGCCACAATCGTCTGTATTTCCATAGTGATACCTGCTTACCTC
TCCGTCCACAAGAAATGGAAGTAGATAGTGAAGATGAAAAGGATCCTGAATGGCTAAGAGAAAAAACCAT
TACACAAATTGAAGAGTTTTCTGATGTTAATGAAGGAGAGAAAGAAGTGATGAAACTCTGGAATCTCCAT
GTCATGAAGCATGGGTTTATTGCTGACAATCAAATGAATCATGCCTGTATGCTGTTTGTAGAAAATTATG
GACAGAAAATAATTAAGAAGAATTTATGTCGAAACTTCATGCTTCATCTAGTCAGCATGCATGACTTTAA
TCTTATTAGCATAATGTCAATAGATAAAGCTGTTACCAAGCTCCGTGAAATGCAGCAAAAATTAGAAAAG
GGGGAATCTGCTTCCCCTGCAAACGAAGAAATAACTGAAGAACAAAATGGGACAGCAAATGGATTTAGTG
AAATTAACTCAAAAGAGAAAGCTTTGGAAACAGATAGTGTCTCAGGGGTTTCAAAACAGAGCAAAAAACA
AAAACTCTGAAAAGCTCTAACCCCATGTTATGGACAAACACTGAAATTACATTTTAGGGAATTCATCCTC
TAAGAATTATGTTTTTGTTTTTAATCATATGTTCCAAACAGGCACTGTTAGATGAAGTAAATGATTTCAA
CAAGGATATTTGTATCAGGGTTCTACTTCACTTCATTATGCAGCATTACATGTATATCACTTTTATTGAT
GTCATTAAAACATTCTGTACTTTAAGCATGAAAAGCAATATTTCAAAGTATTTTTAAACTCAACAAATGT
CATCAAATATGTTGAATTGATCTAGAAATTATTTCATATATAAATCAGAATTTTTTTGCATTTATGAACG
GCTGTTTTTCTACTTTGTAATTGTGAGACATTTTCTTGGGGAGGGAAAATTGGAATGGTTCCCTTTTTTA
GAAATTGAAGTGGTCTTCATATGTCAACTACAGAAAAGGAAAAAAATAGAAATTGAAGGATTTTTATGAA
ATTATATTGCATTACTATTTGCAGTCAAACTTTGATCCTTGTTTTTGAAATCATTTGTCAATTCGGAATG
AAAAATTATAATGTAATTTTACATTACATAAGTTCCTTTTACAATTAAAAAATAGCACTTCTTCATCTTA
TGCCTGTTTGAGAAGATATTAAATTTTCACATTGTTGACAGTGAAATGCTATGTTGGTTTATAAGATTAC
AGACCATTTGTTTTCATGTGGATAATTTTAGTGCATTGCTCACCCGGTATGTTTTTTTTTTTTAACTTGA
ACATTTTGCTTGTTTTGTTTTTCTTTTTTAATTAGATAATCACACGGAAAATTAAGCTGTTCATATCTTT
AAATTAGGATTGCAAACCAAGGAAAGAACGCATTTGAGATTTTAAGATGTCACTTATAAGGGGAGAAGTG
TTCTTAAAAAGTCAACCAGAAAACTGTTATGCCTTTTATTTGTTTGCAAGGATGTCTTTGTAATGTGTTT
CATGAATAGAATATCCAATAGAGATAAGCTGACTTGAATCATTTTGAGCAATTTTGCCCTGTGTTATATG
TGTTTCACGCACATATTTGCAGTTGGATTTTCTCCAACAGAAAGTGGATTCACTACTGGCACATTAACAA
GCACCAATAGGTTTTTATTCCAACTCCGAGCACTGTGGTTGAGTAACATCACCTCAATTTTTTATTATCC
TTAAAGATATTGCATTTTCATATTCTTTATTTATAAAGGATCAATGCTGCTGTAAATACAGGTATTTTTA
ATTTTAAAATTTCATTCCACCACCATCAGATGCAGTTCCCTATTTTGTTTAATGAAGGGATATATAAGCT
TTCTAATGGTGTCTTCAGAAATTTATAAAATGTAAATACTGATTTGACTGGTCTTTAAGATGTGTTTAAC
TGTGAGGCTATTTAACGAATAGTGTGGATGTGATTTGTCATCCAGTATTAAGTTCTTAGTCATTGATTTT
TGTGTTTAAAAAAAAATAGGAAAGAGGGAAACTGCAGCTTTCATTACAGATTCCTTGATTGGTAAGCTCT
CCAAATGATGAGTTCTAGTAAACTCTGATTTTTGCCTCTGGATAGTAGATCTCGAGCGTTTATCTCGGGC
TTTAATTTGCTAAAGCTGTGCACATATGTAAAAAAAAAAAAAAAAAGATTATTTTAGGGGAGATGTAGGT
GTAGAATTATTGCTTATGTCATTTCTTAAGCAGTTATGCTCTTAATGCTTAAAAGAAGGCTAGCATTGTT
TGCACAAAAAGTTGGTGATTCCCACCCCAAATAGTAATAAAATTACTTCTGTTGAGTAAACTTTTTATGT
CATCGTAAAAGCTGAAAAAATCCCTTTGTTTCTATTTATAAAAAAAGTGCTTTTCTATATGTACCCTTGA
TAACAGATTTTGAAGAAATCCTGTAAGATGATAAAGCATTTGAATGGTACAGTAGATGTAAAAAAAATTC
AGTTTAAAAGAACATTTGTTTTTACATTAAATGTTTATTTGAAATCAAATGATTTTGTACATAAAGTTCA
ATAATATAAAAGCTG
NM_001321207.2 Homo sapiens SUZ12 polycomb repressive complex 2 subunit
(SUZ12), transcript variant 2, mRNA
(SEQ ID NO: 64)
GGAGCGAGGCCAGGGTAGGGTGAGCGGCCTCCGAAGCGGAGCGGGGCTCTGAGGAGACACTTTTTTTTTC
CTCCCTCCTTCCCTCCTCTCCTCCTCCCTTCCCTTCCCCTCTCCTCCCCTCTCTCCTCCTTCCCCCCTCG
GTCCGCCGGAGCCTGCTGGGGCGAGCGGTTGGTATTGCAGGCGCTTGCTCTCCGGGGCCGCCCGGCGGGT
AGCTGGCGGGGGGAGGAGGCAGGAACCGCGATGGCGCCTCAGAAGCACGGCGGTGGGGGAGGGGGCGGCT
CGGGGCCCAGCGCGGGGTCCGGGGGAGGCGGCTTCGGGGGTTCGGCGGCGGTGGCGGCGGCGACGGCTTC
GGGCGGCAAATCCGGCGGCGGGAGCTGTGGAGGGGGTGGCAGTTACTCGGCCTCCTCCTCCTCCTCCGCG
GCGGCAGCGGCGGGGGCTGCGGTGTTACCGGTGAAGAAGCCGAAAATGGAGCACGTCCAGGCTGACCACG
AGCTTTTCCTCCAGGCCTTTGAGAAGCCAACACAGATCTATAGATTTCTTCGAACTCGGAATCTCATAGC
ACCAATATTTTTGCACAGAACTCTTACTTACATGTCTCATCGAAACTCCAGAACAAACATCAAAAGCTTG
TCAGCTCATTTGCAGCTTACGTTTACTGGTTTCTTCCACAAAAATGATAAGCCATCACCAAACTCAGAAA
ATGAACAAAATTCTGTTACCCTGGAAGTCCTGCTTGTGAAAGTTTGCCACAAAAAAAGAAAGGATGTAAG
TTGTCCAATAAGGCAAGTTCCCACAGGTAAAAAGCAGGTGCCTTTGAATCCTGACCTCAATCAAACAAAA
CCCGGAAATTTCCCGTCCCTTGCAGTTTCCAGTAATGAATTTGAACCTAGTAACAGCCATATGGTGAAGT
CTTACTCGTTGCTATTTAGAGTGACTCGTCCAGGAAGAAGAGAGTTTAATGGAATGATTAATGGAGAAAC
CAATGAAAATATTGATGTCAATGAAGAGCTTCCAGCCAGAAGAAAACGAAATCGTGAGGATGGGGAAAAG
ACATTTGTTGCACAAATGACAGTATTTGATAAAAACAGGCGCTTACAGCTTTTAGATGGGGAATATGAAG
TAGCCATGCAGGAAATGGAAGAATGTCCAATAAGCAAGAAAAGAGCAACATGGGAGACTATTCTTGATGG
GAAGAGGCTGCCTCCATTCGAAACATTTTCTCAGGGACCTACGTTGCAGTTCACTCTTCGTTGGACAGGA
GAGACCAATGATAAATCTACGGCTCCTATTGCCAAACCTCTTGCCACTAGAAATTCAGAGAGTCTCCATC
AGGAAAACAAGCCTGGTTCAGTTAAACCTACTCAAACTATTGCTGTTAAAGAATCATTGACTACAGATCT
ACAAACAAGAAAAGAAAAGGATACTCCAAATGAAAACCGACAAAAATTAAGAATATTTTATCAGTTTCTC
TATAACAACAATACAAGGCAACAAACTGAAGCAAGAGATGACCTGCATTGCCCTTGGTGTACTCTGAACT
GCCGCAAACTTTATAGTTTACTCAAGCATCTTAAACTCTGCCATAGCAGATTTATCTTCAACTATGTTTA
TCATCCAAAAGGTGCTAGGATAGATGTTTCTATCAATGAGTGTTATGATGGCTCCTATGCAGGAAATCCT
CAGGATATTCATCGCCAACCTGGATTTGCTTTTAGTCGCAACGGACCAGTTAAGAGAACACCTATCACAC
ATATTCTTGTGTGCAGGCCAAAACGAACAAAAGCAAGCATGTCTGAATTTCTTGAATCTGAAGATGGGGA
AGTAGAACAGCAAAGAACATATAGTAGTGGCCACAATCGTCTGTATTTCCATAGTGATACCTGCTTACCT
CTCCGTCCACAAGAAATGGAAGTAGATAGTGAAGATGAAAAGGATCCTGAATGGCTAAGAGAAAAAACCA
TTACACAAATTGAAGAGTTTTCTGATGTTAATGAAGGAGAGAAAGAAGTGATGAAACTCTGGAATCTCCA
TGTCATGAAGCATGGGTTTATTGCTGACAATCAAATGAATCATGCCTGTATGCTGTTTGTAGAAAATTAT
GGACAGAAAATAATTAAGAAGAATTTATGTCGAAACTTCATGCTTCATCTAGTCAGCATGCATGACTTTA
ATCTTATTAGCATAATGTCAATAGATAAAGCTGTTACCAAGCTCCGTGAAATGCAGCAAAAATTAGAAAA
GGGGGAATCTGCTTCCCCTGCAAACGAAGAAATAACTGAAGAACAAAATGGGACAGCAAATGGATTTAGT
GAAATTAACTCAAAAGAGAAAGCTTTGGAAACAGATAGTGTCTCAGGGGTTTCAAAACAGAGCAAAAAAC
AAAAACTCTGAAAAGCTCTAACCCCATGTTATGGACAAACACTGAAATTACATTTTAGGGAATTCATCCT
CTAAGAATTATGTTTTTGTTTTTAATCATATGTTCCAAACAGGCACTGTTAGATGAAGTAAATGATTTCA
ACAAGGATATTTGTATCAGGGTTCTACTTCACTTCATTATGCAGCATTACATGTATATCACTTTTATTGA
TGTCATTAAAACATTCTGTACTTTAAGCATGAAAAGCAATATTTCAAAGTATTTTTAAACTCAACAAATG
TCATCAAATATGTTGAATTGATCTAGAAATTATTTCATATATAAATCAGAATTTTTTTGCATTTATGAAC
GGCTGTTTTTCTACTTTGTAATTGTGAGACATTTTCTTGGGGAGGGAAAATTGGAATGGTTCCCTTTTTT
AGAAATTGAAGTGGTCTTCATATGTCAACTACAGAAAAGGAAAAAAATAGAAATTGAAGGATTTTTATGA
AATTATATTGCATTACTATTTGCAGTCAAACTTTGATCCTTGTTTTTGAAATCATTTGTCAATTCGGAAT
GAAAAATTATAATGTAATTTTACATTACATAAGTTCCTTTTACAATTAAAAAATAGCACTTCTTCATCTT
ATGCCTGTTTGAGAAGATATTAAATTTTCACATTGTTGACAGTGAAATGCTATGTTGGTTTATAAGATTA
CAGACCATTTGTTTTCATGTGGATAATTTTAGTGCATTGCTCACCCGGTATGTTTTTTTTTTTTAACTTG
AACATTTTGCTTGTTTTGTTTTTCTTTTTTAATTAGATAATCACACGGAAAATTAAGCTGTTCATATCTT
TAAATTAGGATTGCAAACCAAGGAAAGAACGCATTTGAGATTTTAAGATGTCACTTATAAGGGGAGAAGT
GTTCTTAAAAAGTCAACCAGAAAACTGTTATGCCTTTTATTTGTTTGCAAGGATGTCTTTGTAATGTGTT
TCATGAATAGAATATCCAATAGAGATAAGCTGACTTGAATCATTTTGAGCAATTTTGCCCTGTGTTATAT
GTGTTTCACGCACATATTTGCAGTTGGATTTTCTCCAACAGAAAGTGGATTCACTACTGGCACATTAACA
AGCACCAATAGGTTTTTATTCCAACTCCGAGCACTGTGGTTGAGTAACATCACCTCAATTTTTTATTATC
CTTAAAGATATTGCATTTTCATATTCTTTATTTATAAAGGATCAATGCTGCTGTAAATACAGGTATTTTT
AATTTTAAAATTTCATTCCACCACCATCAGATGCAGTTCCCTATTTTGTTTAATGAAGGGATATATAAGC
TTTCTAATGGTGTCTTCAGAAATTTATAAAATGTAAATACTGATTTGACTGGTCTTTAAGATGTGTTTAA
CTGTGAGGCTATTTAACGAATAGTGTGGATGTGATTTGTCATCCAGTATTAAGTTCTTAGTCATTGATTT
TTGTGTTTAAAAAAAAATAGGAAAGAGGGAAACTGCAGCTTTCATTACAGATTCCTTGATTGGTAAGCTC
TCCAAATGATGAGTTCTAGTAAACTCTGATTTTTGCCTCTGGATAGTAGATCTCGAGCGTTTATCTCGGG
CTTTAATTTGCTAAAGCTGTGCACATATGTAAAAAAAAAAAAAAAAAGATTATTTTAGGGGAGATGTAGG
TGTAGAATTATTGCTTATGTCATTTCTTAAGCAGTTATGCTCTTAATGCTTAAAAGAAGGCTAGCATTGT
TTGCACAAAAAGTTGGTGATTCCCACCCCAAATAGTAATAAAATTACTTCTGTTGAGTAAACTTTTTATG
TCATCGTAAAAGCTGAAAAAATCCCTTTGTTTCTATTTATAAAAAAAGTGCTTTTCTATATGTACCCTTG
ATAACAGATTTTGAAGAAATCCTGTAAGATGATAAAGCATTTGAATGGTACAGTAGATGTAAAAAAAATT
CAGTTTAAAAGAACATTTGTTTTTACATTAAATGTTTATTTGAAATCAAATGATTTTGTACATAAAGTTC
AATAATATAAAAGCTG
NM_003797.5 Homo sapiens embryonic ectoderm development (EED), transcript
variant 1, mRNA
(SEQ ID NO: 65)
ATTCCACAGACTTTCGCTCCCTAGCAGCGGGTCGGAGATCGAAGGAACGGGCCAATTGCGGCTGAAACGT
CTTTGGAAGGAGGAAGGGGGTGAGGGAGCATCCCTTTGAGTTTCGCCTCTTCTCGAGGCGGTGGTGGGAA
GGGAGACATACTTAATACTGCCCTCTTAATCCAACGGACCTTACATCGTGTAGACTGCCGGGAGGGCGGC
GGGAAAAGGGCAAGACGGGAGTTGGGGAAGGGAAGGAGCCAGGAAGCCGCGCGGGAGGGCGCGCGCGCGC
GCCCCTTTTTCAGCAGTGTGGCGGGGTCGCACGCACGCCCGCCTCGGCGGCTGGGCGCGATTTGCGACAG
TGGGGGGGGCGGTGGAGGTGGCGGCGGCAGCGGCAACTTTGCGGCAAGCTCGGGCCGGGCTTGCTTGACG
GCGGTGTGGCGGAGGCCCCGCCCCAGGCGGCAGGAACCTGGAGGGAGGCGGAGGAATATGTCCGAGAGGG
AAGTGTCGACTGCGCCGGCGGGAACAGACATGCCTGCGGCCAAGAAGCAGAAGCTGAGCAGTGACGAGAA
CAGCAATCCAGACCTCTCTGGAGACGAGAATGATGACGCTGTCAGTATAGAAAGTGGTACAAACACTGAA
CGCCCTGATACACCTACAAACACGCCAAATGCACCTGGAAGGAAAAGTTGGGGAAAGGGAAAATGGAAGT
CAAAGAAATGCAAATATTCTTTCAAATGTGTAAATAGTCTCAAGGAAGATCATAACCAACCATTGTTTGG
AGTTCAGTTTAACTGGCACAGTAAAGAAGGAGATCCATTAGTGTTTGCAACTGTAGGAAGCAACAGAGTT
ACCTTGTATGAATGTCATTCACAAGGAGAAATCCGGTTGTTGCAATCTTACGTGGATGCTGATGCTGATG
AAAACTTTTACACTTGTGCATGGACCTATGATAGCAATACGAGCCATCCTCTGCTGGCTGTAGCTGGATC
TAGAGGCATAATTAGGATAATAAATCCTATAACAATGCAGTGTATAAAGCACTATGTTGGCCATGGAAAT
GCTATCAATGAGCTGAAATTCCATCCAAGAGATCCAAATCTTCTCCTGTCAGTAAGTAAAGATCATGCTT
TACGATTATGGAATATCCAGACGGACACTCTGGTGGCAATATTTGGAGGCGTAGAAGGGCACAGAGATGA
AGTTCTAAGTGCTGATTATGATCTTTTGGGTGAAAAAATAATGTCCTGTGGTATGGATCATTCTCTTAAA
CTTTGGAGGATCAATTCAAAGAGAATGATGAATGCAATTAAGGAATCTTATGATTATAATCCAAATAAAA
CTAACAGGCCATTTATTTCTCAGAAAATCCATTTTCCTGATTTTTCTACCAGAGACATACATAGGAATTA
TGTTGATTGTGTGCGATGGTTAGGCGATTTGATACTTTCTAAGTCTTGTGAAAATGCCATTGTGTGCTGG
AAACCTGGCAAGATGGAAGATGATATAGATAAAATTAAACCCAGTGAATCTAATGTGACTATTCTTGGGC
GATTTGATTACAGCCAGTGTGACATTTGGTACATGAGGTTTTCTATGGATTTCTGGCAAAAGATGCTTGC
ATTGGGCAATCAAGTTGGCAAACTTTATGTTTGGGATTTAGAAGTAGAAGATCCTCATAAAGCCAAATGT
ACAACACTGACTCATCATAAATGTGGTGCTGCTATTCGACAAACCAGTTTTAGCAGGGATAGCAGCATTC
TTATAGCTGTTTGTGATGATGCCAGTATTTGGCGCTGGGATCGACTTCGATAAAATACTTTTGCCTAATC
AAAATTAGAGTGTGTTTGTTGTCTGTGTAAAATAGAATTAATGTATCTTGCTAGTAAGGGCACGTAGAGC
ATTTAGAGTTGTCTTTCAGCATTCAATCAGGCTGAGCTGAATGTAGTGATGTTTACATTGTTTACATTCT
TTGTACTGTCTTCCTGCTCAGACTCTACTGCTTTTAATAAAAATTTATTTTTGTAAAGCTGTGTGTTTAG
TTACTTTCATTGTGGTGAAAAAAAGTTAAAAGTAATAAAATTATGCCTTATCTTTTTA
NM_001308007.2 Homo sapiens embryonic ectoderm development (EED),
transcript variant 3, mRNA
(SEQ ID NO: 66)
ATTCCACAGACTTTCGCTCCCTAGCAGCGGGTCGGAGATCGAAGGAACGGGCCAATTGCGGCTGAAACGT
CTTTGGAAGGAGGAAGGGGGTGAGGGAGCATCCCTTTGAGTTTCGCCTCTTCTCGAGGCGGTGGTGGGAA
GGGAGACATACTTAATACTGCCCTCTTAATCCAACGGACCTTACATCGTGTAGACTGCCGGGAGGGCGGC
GGGAAAAGGGCAAGACGGGAGTTGGGGAAGGGAAGGAGCCAGGAAGCCGCGCGGGAGGGCGCGCGCGCGC
GCCCCTTTTTCAGCAGTGTGGCGGGGTCGCACGCACGCCCGCCTCGGCGGCTGGGCGCGATTTGCGACAG
TGGGGGGGGCGGTGGAGGTGGCGGCGGCAGCGGCAACTTTGCGGCAAGCTCGGGCCGGGCTTGCTTGACG
GCGGTGTGGCGGAGGCCCCGCCCCAGGCGGCAGGAACCTGGAGGGAGGCGGAGGAATATGTCCGAGAGGG
AAGTGTCGACTGCGCCGGCGGGAACAGACATGCCTGCGGCCAAGAAGCAGAAGCTGAGCAGTGACGAGAA
CAGCAATCCAGACCTCTCTGGAGACGAGAATGATGACGCTGTCAGTATAGAAAGTGGTACAAACACTGAA
CGCCCTGATACACCTACAAACACGCCAAATGCACCTGGAAGGAAAAGTTGGGGAAAGGGAAAATGGAAGT
CAAAGAAATGCAAATATTCTTTCAAATGTGTAAATAGTCTCAAGGAAGATCATAACCAACCATTGTTTGG
AGTTCAGTTTAACTGGCACAGTAAAGAAGGAGATCCATTAGTGTTTGCAACTGTAGGAAGCAACAGAGTT
ACCTTGTATGAATGTCATTCACAAGGAGAAATCCGGTTGTTGCAATCTTACGTGGATGCTGATGCTGATG
AAAACTTTTACACTTGTGCATGGACCTATGATAGCAATACGAGCCATCCTCTGCTGGCTGTAGCTGGATC
TAGAGGCATAATTAGGATAATAAATCCTATAACAATGCAGTGTATAAAGCACTATGTTGGCCATGGAAAT
GCTATCAATGAGCTGAAATTCCATCCAAGAGATCCAAATCTTCTCCTGTCAGTAAGTAAAGATCATGCTT
TACGATTATGGAATATCCAGACGGACACTCTGGTGGCAATATTTGGAGGCGTAGAAGGGCACAGAGATGA
AGTTCTAAGTGCTGATTATGATCTTTTGGGTGAAAAAATAATGTCCTGTGGTATGGATCATTCTCTTAAA
CTTTGGAGGATCAATTCAAAGAGAATGATGAATGCAATTAAGGAATCTTATGATTATAATCCAAATAAAA
CTAACAGGCCATTTATTTCTCAGAAAATCCATTTTCCTGATTTTTCTACCAGAGACATACATAGGAATTA
TGTTGATTGTGTGCGATGGTTAGGCGATTTGATACTTTCTAAGAGTGGCCGTGCCATTTTACATTCCCAC
CAGCAATGTATGAGAGATCCAGTGTCTCCGAATCTTCGCCAGCATTTGTCTTGTGAAAATGCCATTGTGT
GCTGGAAACCTGGCAAGATGGAAGATGATATAGATAAAATTAAACCCAGTGAATCTAATGTGACTATTCT
TGGGCGATTTGATTACAGCCAGTGTGACATTTGGTACATGAGGTTTTCTATGGATTTCTGGCAAAAGATG
CTTGCATTGGGCAATCAAGTTGGCAAACTTTATGTTTGGGATTTAGAAGTAGAAGATCCTCATAAAGCCA
AATGTACAACACTGACTCATCATAAATGTGGTGCTGCTATTCGACAAACCAGTTTTAGCAGGGATAGCAG
CATTCTTATAGCTGTTTGTGATGATGCCAGTATTTGGCGCTGGGATCGACTTCGATAAAATACTTTTGCC
TAATCAAAATTAGAGTGTGTTTGTTGTCTGTGTAAAATAGAATTAATGTATCTTGCTAGTAAGGGCACGT
AGAGCATTTAGAGTTGTCTTTCAGCATTCAATCAGGCTGAGCTGAATGTAGTGATGTTTACATTGTTTAC
ATTCTTTGTACTGTCTTCCTGCTCAGACTCTACTGCTTTTAATAAAAATTTATTTTTGTAAAGCTGTGTG
TTTAGTTACTTTCATTGTGGTGAAAAAAAGTTAAAAGTAATAAAATTATGCCTTATCTTTTTA
NM_001330334.2 Homo sapiens embryonic ectoderm development (EED),
transcript variant 4, mRNA
(SEQ ID NO: 67)
ATTCCACAGACTTTCGCTCCCTAGCAGCGGGTCGGAGATCGAAGGAACGGGCCAATTGCGGCTGAAACGT
CTTTGGAAGGAGGAAGGGGGTGAGGGAGCATCCCTTTGAGTTTCGCCTCTTCTCGAGGCGGTGGTGGGAA
GGGAGACATACTTAATACTGCCCTCTTAATCCAACGGACCTTACATCGTGTAGACTGCCGGGAGGGCGGC
GGGAAAAGGGCAAGACGGGAGTTGGGGAAGGGAAGGAGCCAGGAAGCCGCGCGGGAGGGCGCGCGCGCGC
GCCCCTTTTTCAGCAGTGTGGCGGGGTCGCACGCACGCCCGCCTCGGCGGCTGGGCGCGATTTGCGACAG
TGGGGGGGGCGGTGGAGGTGGCGGCGGCAGCGGCAACTTTGCGGCAAGCTCGGGCCGGGCTTGCTTGACG
GCGGTGTGGCGGAGGCCCCGCCCCAGGCGGCAGGAACCTGGAGGGAGGCGGAGGAATATGTCCGAGAGGG
AAGTGTCGACTGCGCCGGCGGGAACAGACATGCCTGCGGCCAAGAAGCAGAAGCTGAGCAGTGACGAGAA
CAGCAATCCAGACCTCTCTGGAGACGAGAATGATGACGCTGTCAGTATAGAAAGTGGTACAAACACTGAA
CGCCCTGATACACCTACAAACACGCCAAATGCACCTGGAAGGAAAAGTTGGGGAAAGGGAAAATGGAAGT
CAAAGAAATGCAAATATTCTTTCAAATGTGTAAATAGTCTCAAGGAAGATCATAACCAACCATTGTTTGG
AGTTCAGTTTAACTGGCACAGTAAAGAAGGAGATCCATTAGTGTTTGCAACTGTAGGAAGCAACAGAGTT
ACCTTGTATGAATGTCATTCACAAGGAGAAATCCGGTTGTTGCAATCTTACGTGGATGCTGATGCTGATG
AAAACTTTTACACTTGTGCATGGACCTATGATAGCAATACGAGCCATCCTCTGCTGGCTGTAGCTGGATC
TAGAGGCATAATTAGGATAATAAATCCTATAACAATGCAGTGTATAAAGCACTATGTTGGCCATGGAAAT
GCTATCAATGAGCTGAAATTCCATCCAAGAGATCCAAATCTTCTCCTGTCAGTAAGTAAAGATCATGCTT
TACGATTATGGAATATCCAGACGGACACTCTGGTGGCAATATTTGGAGGCGTAGAAGGGCACAGAGATGA
AGTTCTAAGTGCTTCTTGTGAAAATGCCATTGTGTGCTGGAAACCTGGCAAGATGGAAGATGATATAGAT
AAAATTAAACCCAGTGAATCTAATGTGACTATTCTTGGGCGATTTGATTACAGCCAGTGTGACATTTGGT
ACATGAGGTTTTCTATGGATTTCTGGCAAAAGATGCTTGCATTGGGCAATCAAGTTGGCAAACTTTATGT
TTGGGATTTAGAAGTAGAAGATCCTCATAAAGCCAAATGTACAACACTGACTCATCATAAATGTGGTGCT
GCTATTCGACAAACCAGTTTTAGCAGGGATAGCAGCATTCTTATAGCTGTTTGTGATGATGCCAGTATTT
GGCGCTGGGATCGACTTCGATAAAATACTTTTGCCTAATCAAAATTAGAGTGTGTTTGTTGTCTGTGTAA
AATAGAATTAATGTATCTTGCTAGTAAGGGCACGTAGAGCATTTAGAGTTGTCTTTCAGCATTCAATCAG
GCTGAGCTGAATGTAGTGATGTTTACATTGTTTACATTCTTTGTACTGTCTTCCTGCTCAGACTCTACTG
CTTTTAATAAAAATTTATTTTTGTAAAGCTGTGTGTTTAGTTACTTTCATTGTGGTGAAAAAAAGTTAAA
AGTAATAAAATTATGCCTTATCTTTTTA
NM_001321079.2 Homo sapiens enhancer of zeste 1 polycomb repressive
complex 2 subunit (EZH1), transcript variant 2, mRNA
(SEQ ID NO: 68)
AGCAGCGGGACCCGCGGCTCGGGATGGAGGATTACAGCAAGATGGAAATACCAAATCCCCCTACCTCCAA
ATGTATCACTTACTGGAAAAGAAAAGTGAAATCTGAATACATGCGACTTCGACAACTTAAACGGCTTCAG
GCAAATATGGGTGCAAAGGCTTTGTATGTGGCAAATTTTGCAAAGGTTCAAGAAAAAACCCAGATCCTCA
ATGAAGAATGGAAGAAGCTTCGTGTCCAACCTGTTCAGTCAATGAAGCCTGTGAGTGGACACCCTTTTCT
CAAAAAGTGTACCATAGAGAGCATTTTCCCGGGATTTGCAAGCCAACATATGTTAATGAGGTCACTGAAC
ACAGTTGCATTGGTTCCCATCATGTATTCCTGGTCCCCTCTCCAACAGAACTTTATGGTAGAAGATGAGA
CGGTTTTGTGCAATATTCCCTACATGGGAGATGAAGTGAAAGAAGAAGATGAGACTTTTATTGAGGAGCT
GATCAATAACTATGATGGGAAAGTCCATGGTGAAGAAGAGATGATCCCTGGATCCGTTCTGATTAGTGAT
GCTGTTTTTCTGGAGTTGGTCGATGCCCTGAATCAGTACTCAGATGAGGAGGAGGAAGGGCACAATGACA
CCTCAGATGGAAAGCAGGATGACAGCAAAGAAGATCTGCCAGTAACAAGAAAGAGAAAGCGACATGCTAT
TGAAGGCAACAAAAAGAGTTCCAAGAAACAGTTCCCAAATGACATGATCTTCAGTGCAATTGCCTCAATG
TTCCCTGAGAATGGTGTCCCAGATGACATGAAGGAGAGGTATCGAGAACTAACAGAGATGTCAGACCCCA
ATGCACTTCCCCCTCAGTGCACACCCAACATCGATGGCCCCAATGCCAAGTCTGTGCAGCGGGAGCAATC
TCTGCACTCCTTCCACACACTTTTTTGCCGGCGCTGCTTTAAATACGACTGCTTCCTTCACCCTTTTCAT
GCCACCCCTAATGTATATAAACGCAAGAATAAAGAAATCAAGATTGAACCAGAACCATGTGGCACAGACT
GCTTCCTTTTGCTGGAAGGAGCAAAGGAGTATGCCATGCTCCACAACCCCCGCTCCAAGTGCTCTGGTCG
TCGCCGGAGAAGGCACCACATAGTCAGTGCTTCCTGCTCCAATGCCTCAGCCTCTGCTGTGGCTGAGACT
AAAGAAGGAGACAGTGACAGGGACACAGGCAATGACTGGGCCTCCAGTTCTTCAGAGGCTAACTCTCGCT
GTCAGACTCCCACAAAACAGAAGGCTAGTCCAGCCCCACCTCAACTCTGCGTAGTGGAAGCACCCTCGGA
GCCTGTGGAATGGACTGGGGCTGAAGAATCTCTTTTTCGAGTCTTCCATGGCACCTACTTCAACAACTTC
TGTTCAATAGCCAGGCTTCTGGGGACCAAGACGTGCAAGCAGGTCTTTCAGTTTGCAGTCAAAGAATCAC
TTATCCTGAAGCTGCCAACAGATGAGCTCATGAACCCCTCACAGAAGAAGAAAAGAAAGCACAGATTGTG
GGCTGCACACTGCAGGAAGATTCAGCTGAAGAAAGATAACTCTTCCACACAAGTGTACAACTACCAACCC
TGCGACCACCCAGACCGCCCCTGTGACAGCACCTGCCCCTGCATCATGACTCAGAATTTCTGTGAGAAGT
TCTGCCAGTGCAACCCAGACTGTCAGAATCGTTTCCCTGGCTGTCGCTGTAAGACCCAGTGCAATACCAA
GCAATGTCCTTGCTATCTGGCAGTGCGAGAATGTGACCCTGACCTGTGTCTCACCTGTGGGGCCTCAGAG
CACTGGGACTGCAAGGTGGTTTCCTGTAAAAACTGCAGCATCCAGCGTGGACTTAAGAAGCACCTGCTGC
TGGCCCCCTCTGATGTGGCCGGATGGGGCACCTTCATAAAGGAGTCTGTGCAGAAGAACGAATTCATTTC
TGAATACTGTGGTGAGCTCATCTCTCAGGATGAGGCTGATCGACGCGGAAAGGTCTATGACAAATACATG
TCCAGCTTCCTCTTCAACCTCAATAATGATTTTGTAGTGGATGCTACTCGGAAAGGAAACAAAATTCGAT
TTGCAAATCATTCAGTGAATCCCAACTGTTATGCCAAAGTGGTCATGGTGAATGGAGACCATCGGATTGG
GATCTTTGCCAAGAGGGCAATTCAAGCTGGCGAAGAGCTCTTCTTTGATTACAGGTACAGCCAAGCTGAT
GCTCTCAAGTACGTGGGGATCGAGAGGGAGACCGACGTCCTTTAGCCCTCCCAGGCCCCACGGCAGCACT
TATGGTAGCGGCACTGTCTTGGCTTTCGTGCTCACACCACTGCTGCTCGAGTCTCCTGCACTGTGTCTCC
CACACTGAGAAACCCCCCAACCCACTCCCTCTGTAGTGAGGCCTCTGCCATGTCCAGAGGGCACAAAACT
GTCTCAATGAGAGGGGAGACAGAGGCAGCTAGGGCTTGGTCTCCCAGGACAGAGAGTTACAGAAATGGGA
GACTGTTTCTCTGGCCTCAGAAGAAGCGAGCACAGGCTGGGGTGGATGACTTATGCGTGATTTCGTGTCG
GCTCCCCAGGCTGTGGCCTCAGGAATCAACTTAGGCAGTTCCCAACAAGCGCTAGCCTGTAATTGTAGCT
TTCCACATCAAGAGTCCTTATGTTATTGGGATGCAGGCAAACCTCTGTGGTCCTAAGACCTGGAGAGGAC
AGGCTAAGTGAAGTGTGGTCCCTGGAGCCTACAAGTGGTCTGGGTTAGAGGCGAGCCTGGCAGGCAGCAC
AGACTGAACTCAGAGGTAGACAGGTCACCTTACTACCTCCTCCCTCGTGGCAGGGCTCAAACTGAAAGAG
TGTGGGTTCTAAGTACAGGCATTCAAGGCTGGGGGAAGGAAAGCTACGCCATCCTTCCTTAGCCAGAGAG
GGAGAACCAGCCAGATGATAGTAGTTAAACTGCTAAGCTTGGGCCCAGGAGGCTTTGAGAAAGCCTTCTC
TGTGTACTCTGGAGATAGATGGAGAAGTGTTTTCAGATTCCTGGGAACAGACACCAGTGCTCCAGCTCCT
CCAAAGTTCTGGCTTAGCAGCTGCAGGCAAGCATTATGCTGCTATTGAAGAAGCATTAGGGGTATGCCTG
GCAGGTGTGAGCATCCTGGCTCGCTGGATTTGTGGGTGTTTTCAGGCCTTCCATTCCCCATAGAGGCAAG
GCCCAATGGCCAGTGTTGCTTATCGCTTCAGGGTAGGTGGGCACAGGCTTGGACTAGAGAGGAGAAAGAT
TGGTGTAATCTGCTTTCCTGTCTGTAGTGCCTGCTGTTTGGAAAGGGTGAGTTAGAATATGTTCCAAGGT
TGGTGAGGGGCTAAATTGCACGCGTTTAGGCTGGCACCCCGTGTGCAGGGCACACTGGCAGAGGGTATCT
GAAGTGGGAGAAGAAGCAGGTAGACCACCTGTCCCAGGCTGTGGTGCCACCCTCTCTGGCATTCATGCAG
AGCAAAGCACTTTAACCATTTCTTTTAAAAGGTCTATAGATTGGGGTAGAGTTTGGCCTAAGGTCTCTAG
GGTCCCTGCCTAAATCCCACTCCTGAGGGAGGGGGAAGAAGAGAGGGTGGGAGATTCTCCTCCAGTCCTG
TCTCATCTCCTGGGAGAGGCAGACGAGTGAGTTTCACACAGAAGAATTTCATGTGAATGGGGCCAGCAAG
AGCTGCCCTGTGTCCATGGTGGGTGTGCCGGGCTGGCTGGGAACAAGGAGCAGTATGTTGAGTAGAAAGG
GTGTGGGCGGGTATAGATTGGCCTGGGAGTGTTACAGTAGGGAGCAGGCTTCTCCCTTCTTTCTGGGACT
CAGAGCCCCGCTTCTTCCCACTCCACTTGTTGTCCCATGAAGGAAGAAGTGGGGTTCCTCCTGACCCAGC
TGCCTCTTACGGTTTGGTATGGGACATGCACACACACTCACATGCTCTCACTCACCACACTGGAGGGCAC
ACACGTACCCCGCACCCAGCAACTCCTGACAGAAAGCTCCTCCCACCCAAATGGGCCAGGCCCCAGCATG
ATCCTGAAATCTGCATCCGCCGTGGTTTGTATTCATTGTGCATATCAGGGATACCCTCAAGCTGGACTGT
GGGTTCCAAATTACTCATAGAGGAGAAAACCAGAGAAAGATGAAGAGGAGGAGTTAGGTCTATTTGAAAT
GCCAGGGGCTCGCTGTGAGGAATAGGTGAAAAAAAACTTTTCACCAGCCTTTGAGAGACTAGACTGACCC
CACCCTTCCTTCAGTGAGCAGAATCACTGTGGTCAGTCTCCTGTCCCAGCTTCAGTTCATGAATACTCCT
GTTCCTCCAGTTTCCCATCCTTTGTCCCTGCTGTCCCCCACTTTTAAAGATGGGTCTCAACCCCTCCCCA
CCACGTCATGATGGATGGGGCAAGGTGGTGGGGACTAGGGGAGCCTGGTATACATGCGGCTTCATTGCCA
ATAAATTTCATGCACTTTAAAGTCCTGTGGCTTGTGACCTCTTAATAAAGTGTTAGAATCCA
NM_001321082.2 Homo sapiens enhancer of zeste 1 polycomb repressive
complex 2 subunit (EZH1), transcript variant 3, mRNA
(SEQ ID NO: 69)
AGCAGCGGGACCCGCGGCTCGGGATGGAGGCTGGACACCTGTTCTGCTGTTGTGTCCTGCCATTCTCCTG
AAGAACAGAGGCACACTGTAAAACCCAACACTTCCCCTTGCATTCTATAAGATTACAGCAAGATGGAAAT
ACCAAATCCCCCTACCTCCAAATGTATCACTTACTGGAAAAGAAAAGTGAAATCTGAATACATGCGACTT
CGACAACTTAAACGGCTTCAGGCAAATATGGGTGCAAAGGCTTTGTATGTGGCAAATTTTGCAAAGGTTC
AAGAAAAAACCCAGATCCTCAATGAAGAATGGAAGAAGCTTCGTGTCCAACCTGTTCAGTCAATGAAGCC
TGTGAGTGGACACCCTTTTCTCAAAAAGGTAGAAGATGAGACGGTTTTGTGCAATATTCCCTACATGGGA
GATGAAGTGAAAGAAGAAGATGAGACTTTTATTGAGGAGCTGATCAATAACTATGATGGGAAAGTCCATG
GTGAAGAAGAGATGATCCCTGGATCCGTTCTGATTAGTGATGCTGTTTTTCTGGAGTTGGTCGATGCCCT
GAATCAGTACTCAGATGAGGAGGAGGAAGGGCACAATGACACCTCAGATGGAAAGCAGGATGACAGCAAA
GAAGATCTGCCAGTAACAAGAAAGAGAAAGCGACATGCTATTGAAGGCAACAAAAAGAGTTCCAAGAAAC
AGTTCCCAAATGACATGATCTTCAGTGCAATTGCCTCAATGTTCCCTGAGAATGGTGTCCCAGATGACAT
GAAGGAGAGGTATCGAGAACTAACAGAGATGTCAGACCCCAATGCACTTCCCCCTCAGTGCACACCCAAC
ATCGATGGCCCCAATGCCAAGTCTGTGCAGCGGGAGCAATCTCTGCACTCCTTCCACACACTTTTTTGCC
GGCGCTGCTTTAAATACGACTGCTTCCTTCACCCTTTTCATGCCACCCCTAATGTATATAAACGCAAGAA
TAAAGAAATCAAGATTGAACCAGAACCATGTGGCACAGACTGCTTCCTTTTGCTGGAAGGAGCAAAGGAG
TATGCCATGCTCCACAACCCCCGCTCCAAGTGCTCTGGTCGTCGCCGGAGAAGGCACCACATAGTCAGTG
CTTCCTGCTCCAATGCCTCAGCCTCTGCTGTGGCTGAGACTAAAGAAGGAGACAGTGACAGGGACACAGG
CAATGACTGGGCCTCCAGTTCTTCAGAGGCTAACTCTCGCTGTCAGACTCCCACAAAACAGAAGGCTAGT
CCAGCCCCACCTCAACTCTGCGTAGTGGAAGCACCCTCGGAGCCTGTGGAATGGACTGGGGCTGAAGAAT
CTCTTTTTCGAGTCTTCCATGGCACCTACTTCAACAACTTCTGTTCAATAGCCAGGCTTCTGGGGACCAA
GACGTGCAAGCAGGTCTTTCAGTTTGCAGTCAAAGAATCACTTATCCTGAAGCTGCCAACAGATGAGCTC
ATGAACCCCTCACAGAAGAAGAAAAGAAAGCACAGATTGTGGGCTGCACACTGCAGGAAGATTCAGCTGA
AGAAAGATAACTCTTCCACACAAGTGTACAACTACCAACCCTGCGACCACCCAGACCGCCCCTGTGACAG
CACCTGCCCCTGCATCATGACTCAGAATTTCTGTGAGAAGTTCTGCCAGTGCAACCCAGACTGTCAGAAT
CGTTTCCCTGGCTGTCGCTGTAAGACCCAGTGCAATACCAAGCAATGTCCTTGCTATCTGGCAGTGCGAG
AATGTGACCCTGACCTGTGTCTCACCTGTGGGGCCTCAGAGCACTGGGACTGCAAGGTGGTTTCCTGTAA
AAACTGCAGCATCCAGCGTGGACTTAAGAAGCACCTGCTGCTGGCCCCCTCTGATGTGGCCGGATGGGGC
ACCTTCATAAAGGAGTCTGTGCAGAAGAACGAATTCATTTCTGAATACTGTGGTGAGCTCATCTCTCAGG
ATGAGGCTGATCGACGCGGAAAGGTCTATGACAAATACATGTCCAGCTTCCTCTTCAACCTCAATAATGA
TTTTGTAGTGGATGCTACTCGGAAAGGAAACAAAATTCGATTTGCAAATCATTCAGTGAATCCCAACTGT
TATGCCAAAGTGGTCATGGTGAATGGAGACCATCGGATTGGGATCTTTGCCAAGAGGGCAATTCAAGCTG
GCGAAGAGCTCTTCTTTGATTACAGGTACAGCCAAGCTGATGCTCTCAAGTACGTGGGGATCGAGAGGGA
GACCGACGTCCTTTAGCCCTCCCAGGCCCCACGGCAGCACTTATGGTAGCGGCACTGTCTTGGCTTTCGT
GCTCACACCACTGCTGCTCGAGTCTCCTGCACTGTGTCTCCCACACTGAGAAACCCCCCAACCCACTCCC
TCTGTAGTGAGGCCTCTGCCATGTCCAGAGGGCACAAAACTGTCTCAATGAGAGGGGAGACAGAGGCAGC
TAGGGCTTGGTCTCCCAGGACAGAGAGTTACAGAAATGGGAGACTGTTTCTCTGGCCTCAGAAGAAGCGA
GCACAGGCTGGGGTGGATGACTTATGCGTGATTTCGTGTCGGCTCCCCAGGCTGTGGCCTCAGGAATCAA
CTTAGGCAGTTCCCAACAAGCGCTAGCCTGTAATTGTAGCTTTCCACATCAAGAGTCCTTATGTTATTGG
GATGCAGGCAAACCTCTGTGGTCCTAAGACCTGGAGAGGACAGGCTAAGTGAAGTGTGGTCCCTGGAGCC
TACAAGTGGTCTGGGTTAGAGGCGAGCCTGGCAGGCAGCACAGACTGAACTCAGAGGTAGACAGGTCACC
TTACTACCTCCTCCCTCGTGGCAGGGCTCAAACTGAAAGAGTGTGGGTTCTAAGTACAGGCATTCAAGGC
TGGGGGAAGGAAAGCTACGCCATCCTTCCTTAGCCAGAGAGGGAGAACCAGCCAGATGATAGTAGTTAAA
CTGCTAAGCTTGGGCCCAGGAGGCTTTGAGAAAGCCTTCTCTGTGTACTCTGGAGATAGATGGAGAAGTG
TTTTCAGATTCCTGGGAACAGACACCAGTGCTCCAGCTCCTCCAAAGTTCTGGCTTAGCAGCTGCAGGCA
AGCATTATGCTGCTATTGAAGAAGCATTAGGGGTATGCCTGGCAGGTGTGAGCATCCTGGCTCGCTGGAT
TTGTGGGTGTTTTCAGGCCTTCCATTCCCCATAGAGGCAAGGCCCAATGGCCAGTGTTGCTTATCGCTTC
AGGGTAGGTGGGCACAGGCTTGGACTAGAGAGGAGAAAGATTGGTGTAATCTGCTTTCCTGTCTGTAGTG
CCTGCTGTTTGGAAAGGGTGAGTTAGAATATGTTCCAAGGTTGGTGAGGGGCTAAATTGCACGCGTTTAG
GCTGGCACCCCGTGTGCAGGGCACACTGGCAGAGGGTATCTGAAGTGGGAGAAGAAGCAGGTAGACCACC
TGTCCCAGGCTGTGGTGCCACCCTCTCTGGCATTCATGCAGAGCAAAGCACTTTAACCATTTCTTTTAAA
AGGTCTATAGATTGGGGTAGAGTTTGGCCTAAGGTCTCTAGGGTCCCTGCCTAAATCCCACTCCTGAGGG
AGGGGGAAGAAGAGAGGGTGGGAGATTCTCCTCCAGTCCTGTCTCATCTCCTGGGAGAGGCAGACGAGTG
AGTTTCACACAGAAGAATTTCATGTGAATGGGGCCAGCAAGAGCTGCCCTGTGTCCATGGTGGGTGTGCC
GGGCTGGCTGGGAACAAGGAGCAGTATGTTGAGTAGAAAGGGTGTGGGCGGGTATAGATTGGCCTGGGAG
TGTTACAGTAGGGAGCAGGCTTCTCCCTTCTTTCTGGGACTCAGAGCCCCGCTTCTTCCCACTCCACTTG
TTGTCCCATGAAGGAAGAAGTGGGGTTCCTCCTGACCCAGCTGCCTCTTACGGTTTGGTATGGGACATGC
ACACACACTCACATGCTCTCACTCACCACACTGGAGGGCACACACGTACCCCGCACCCAGCAACTCCTGA
CAGAAAGCTCCTCCCACCCAAATGGGCCAGGCCCCAGCATGATCCTGAAATCTGCATCCGCCGTGGTTTG
TATTCATTGTGCATATCAGGGATACCCTCAAGCTGGACTGTGGGTTCCAAATTACTCATAGAGGAGAAAA
CCAGAGAAAGATGAAGAGGAGGAGTTAGGTCTATTTGAAATGCCAGGGGCTCGCTGTGAGGAATAGGTGA
AAAAAAACTTTTCACCAGCCTTTGAGAGACTAGACTGACCCCACCCTTCCTTCAGTGAGCAGAATCACTG
TGGTCAGTCTCCTGTCCCAGCTTCAGTTCATGAATACTCCTGTTCCTCCAGTTTCCCATCCTTTGTCCCT
GCTGTCCCCCACTTTTAAAGATGGGTCTCAACCCCTCCCCACCACGTCATGATGGATGGGGCAAGGTGGT
GGGGACTAGGGGAGCCTGGTATACATGCGGCTTCATTGCCAATAAATTTCATGCACTTTAAAGTCCTGTG
GCTTGTGACCTCTTAATAAAGTGTTAGAATCCA
NM_001991.5 Homo sapiens enhancer of zeste 1 polycomb repressive complex 2
subunit (EZH1), transcript variant 1, mRNA
(SEQ ID NO: 70)
AGCAGCGGGACCCGCGGCTCGGGATGGAGGCTGGACACCTGTTCTGCTGTTGTGTCCTGCCATTCTCCTG
AAGAACAGAGGCACACTGTAAAACCCAACACTTCCCCTTGCATTCTATAAGATTACAGCAAGATGGAAAT
ACCAAATCCCCCTACCTCCAAATGTATCACTTACTGGAAAAGAAAAGTGAAATCTGAATACATGCGACTT
CGACAACTTAAACGGCTTCAGGCAAATATGGGTGCAAAGGCTTTGTATGTGGCAAATTTTGCAAAGGTTC
AAGAAAAAACCCAGATCCTCAATGAAGAATGGAAGAAGCTTCGTGTCCAACCTGTTCAGTCAATGAAGCC
TGTGAGTGGACACCCTTTTCTCAAAAAGTGTACCATAGAGAGCATTTTCCCGGGATTTGCAAGCCAACAT
ATGTTAATGAGGTCACTGAACACAGTTGCATTGGTTCCCATCATGTATTCCTGGTCCCCTCTCCAACAGA
ACTTTATGGTAGAAGATGAGACGGTTTTGTGCAATATTCCCTACATGGGAGATGAAGTGAAAGAAGAAGA
TGAGACTTTTATTGAGGAGCTGATCAATAACTATGATGGGAAAGTCCATGGTGAAGAAGAGATGATCCCT
GGATCCGTTCTGATTAGTGATGCTGTTTTTCTGGAGTTGGTCGATGCCCTGAATCAGTACTCAGATGAGG
AGGAGGAAGGGCACAATGACACCTCAGATGGAAAGCAGGATGACAGCAAAGAAGATCTGCCAGTAACAAG
AAAGAGAAAGCGACATGCTATTGAAGGCAACAAAAAGAGTTCCAAGAAACAGTTCCCAAATGACATGATC
TTCAGTGCAATTGCCTCAATGTTCCCTGAGAATGGTGTCCCAGATGACATGAAGGAGAGGTATCGAGAAC
TAACAGAGATGTCAGACCCCAATGCACTTCCCCCTCAGTGCACACCCAACATCGATGGCCCCAATGCCAA
GTCTGTGCAGCGGGAGCAATCTCTGCACTCCTTCCACACACTTTTTTGCCGGCGCTGCTTTAAATACGAC
TGCTTCCTTCACCCTTTTCATGCCACCCCTAATGTATATAAACGCAAGAATAAAGAAATCAAGATTGAAC
CAGAACCATGTGGCACAGACTGCTTCCTTTTGCTGGAAGGAGCAAAGGAGTATGCCATGCTCCACAACCC
CCGCTCCAAGTGCTCTGGTCGTCGCCGGAGAAGGCACCACATAGTCAGTGCTTCCTGCTCCAATGCCTCA
GCCTCTGCTGTGGCTGAGACTAAAGAAGGAGACAGTGACAGGGACACAGGCAATGACTGGGCCTCCAGTT
CTTCAGAGGCTAACTCTCGCTGTCAGACTCCCACAAAACAGAAGGCTAGTCCAGCCCCACCTCAACTCTG
CGTAGTGGAAGCACCCTCGGAGCCTGTGGAATGGACTGGGGCTGAAGAATCTCTTTTTCGAGTCTTCCAT
GGCACCTACTTCAACAACTTCTGTTCAATAGCCAGGCTTCTGGGGACCAAGACGTGCAAGCAGGTCTTTC
AGTTTGCAGTCAAAGAATCACTTATCCTGAAGCTGCCAACAGATGAGCTCATGAACCCCTCACAGAAGAA
GAAAAGAAAGCACAGATTGTGGGCTGCACACTGCAGGAAGATTCAGCTGAAGAAAGATAACTCTTCCACA
CAAGTGTACAACTACCAACCCTGCGACCACCCAGACCGCCCCTGTGACAGCACCTGCCCCTGCATCATGA
CTCAGAATTTCTGTGAGAAGTTCTGCCAGTGCAACCCAGACTGTCAGAATCGTTTCCCTGGCTGTCGCTG
TAAGACCCAGTGCAATACCAAGCAATGTCCTTGCTATCTGGCAGTGCGAGAATGTGACCCTGACCTGTGT
CTCACCTGTGGGGCCTCAGAGCACTGGGACTGCAAGGTGGTTTCCTGTAAAAACTGCAGCATCCAGCGTG
GACTTAAGAAGCACCTGCTGCTGGCCCCCTCTGATGTGGCCGGATGGGGCACCTTCATAAAGGAGTCTGT
GCAGAAGAACGAATTCATTTCTGAATACTGTGGTGAGCTCATCTCTCAGGATGAGGCTGATCGACGCGGA
AAGGTCTATGACAAATACATGTCCAGCTTCCTCTTCAACCTCAATAATGATTTTGTAGTGGATGCTACTC
GGAAAGGAAACAAAATTCGATTTGCAAATCATTCAGTGAATCCCAACTGTTATGCCAAAGTGGTCATGGT
GAATGGAGACCATCGGATTGGGATCTTTGCCAAGAGGGCAATTCAAGCTGGCGAAGAGCTCTTCTTTGAT
TACAGGTACAGCCAAGCTGATGCTCTCAAGTACGTGGGGATCGAGAGGGAGACCGACGTCCTTTAGCCCT
CCCAGGCCCCACGGCAGCACTTATGGTAGCGGCACTGTCTTGGCTTTCGTGCTCACACCACTGCTGCTCG
AGTCTCCTGCACTGTGTCTCCCACACTGAGAAACCCCCCAACCCACTCCCTCTGTAGTGAGGCCTCTGCC
ATGTCCAGAGGGCACAAAACTGTCTCAATGAGAGGGGAGACAGAGGCAGCTAGGGCTTGGTCTCCCAGGA
CAGAGAGTTACAGAAATGGGAGACTGTTTCTCTGGCCTCAGAAGAAGCGAGCACAGGCTGGGGTGGATGA
CTTATGCGTGATTTCGTGTCGGCTCCCCAGGCTGTGGCCTCAGGAATCAACTTAGGCAGTTCCCAACAAG
CGCTAGCCTGTAATTGTAGCTTTCCACATCAAGAGTCCTTATGTTATTGGGATGCAGGCAAACCTCTGTG
GTCCTAAGACCTGGAGAGGACAGGCTAAGTGAAGTGTGGTCCCTGGAGCCTACAAGTGGTCTGGGTTAGA
GGCGAGCCTGGCAGGCAGCACAGACTGAACTCAGAGGTAGACAGGTCACCTTACTACCTCCTCCCTCGTG
GCAGGGCTCAAACTGAAAGAGTGTGGGTTCTAAGTACAGGCATTCAAGGCTGGGGGAAGGAAAGCTACGC
CATCCTTCCTTAGCCAGAGAGGGAGAACCAGCCAGATGATAGTAGTTAAACTGCTAAGCTTGGGCCCAGG
AGGCTTTGAGAAAGCCTTCTCTGTGTACTCTGGAGATAGATGGAGAAGTGTTTTCAGATTCCTGGGAACA
GACACCAGTGCTCCAGCTCCTCCAAAGTTCTGGCTTAGCAGCTGCAGGCAAGCATTATGCTGCTATTGAA
GAAGCATTAGGGGTATGCCTGGCAGGTGTGAGCATCCTGGCTCGCTGGATTTGTGGGTGTTTTCAGGCCT
TCCATTCCCCATAGAGGCAAGGCCCAATGGCCAGTGTTGCTTATCGCTTCAGGGTAGGTGGGCACAGGCT
TGGACTAGAGAGGAGAAAGATTGGTGTAATCTGCTTTCCTGTCTGTAGTGCCTGCTGTTTGGAAAGGGTG
AGTTAGAATATGTTCCAAGGTTGGTGAGGGGCTAAATTGCACGCGTTTAGGCTGGCACCCCGTGTGCAGG
GCACACTGGCAGAGGGTATCTGAAGTGGGAGAAGAAGCAGGTAGACCACCTGTCCCAGGCTGTGGTGCCA
CCCTCTCTGGCATTCATGCAGAGCAAAGCACTTTAACCATTTCTTTTAAAAGGTCTATAGATTGGGGTAG
AGTTTGGCCTAAGGTCTCTAGGGTCCCTGCCTAAATCCCACTCCTGAGGGAGGGGGAAGAAGAGAGGGTG
GGAGATTCTCCTCCAGTCCTGTCTCATCTCCTGGGAGAGGCAGACGAGTGAGTTTCACACAGAAGAATTT
CATGTGAATGGGGCCAGCAAGAGCTGCCCTGTGTCCATGGTGGGTGTGCCGGGCTGGCTGGGAACAAGGA
GCAGTATGTTGAGTAGAAAGGGTGTGGGCGGGTATAGATTGGCCTGGGAGTGTTACAGTAGGGAGCAGGC
TTCTCCCTTCTTTCTGGGACTCAGAGCCCCGCTTCTTCCCACTCCACTTGTTGTCCCATGAAGGAAGAAG
TGGGGTTCCTCCTGACCCAGCTGCCTCTTACGGTTTGGTATGGGACATGCACACACACTCACATGCTCTC
ACTCACCACACTGGAGGGCACACACGTACCCCGCACCCAGCAACTCCTGACAGAAAGCTCCTCCCACCCA
AATGGGCCAGGCCCCAGCATGATCCTGAAATCTGCATCCGCCGTGGTTTGTATTCATTGTGCATATCAGG
GATACCCTCAAGCTGGACTGTGGGTTCCAAATTACTCATAGAGGAGAAAACCAGAGAAAGATGAAGAGGA
GGAGTTAGGTCTATTTGAAATGCCAGGGGCTCGCTGTGAGGAATAGGTGAAAAAAAACTTTTCACCAGCC
TTTGAGAGACTAGACTGACCCCACCCTTCCTTCAGTGAGCAGAATCACTGTGGTCAGTCTCCTGTCCCAG
CTTCAGTTCATGAATACTCCTGTTCCTCCAGTTTCCCATCCTTTGTCCCTGCTGTCCCCCACTTTTAAAG
ATGGGTCTCAACCCCTCCCCACCACGTCATGATGGATGGGGCAAGGTGGTGGGGACTAGGGGAGCCTGGT
ATACATGCGGCTTCATTGCCAATAAATTTCATGCACTTTAAAGTCCTGTGGCTTGTGACCTCTTAATAAA
GTGTTAGAATCCA
NM_001321081.2 Homo sapiens enhancer of zeste 1 polycomb repressive
complex 2 subunit (EZH1), transcript variant 4, mRNA
(SEQ ID NO: 71)
AGCAGCGGGACCCGCGGCTCGGGATGGAGGCTGGACACCTGTTCTGCTGTTGTGTCCTGCCATTCTCCTG
AAGAACAGAGGCACACTGTAAAACCCAACACTTCCCCTTGCATTCTATAAGATTACAGCAAGATGGAAAT
ACCAAATCCCCCTACCTCCAAATGTATCACTTACTGGAAAAGAAAAGTGAAATCTGAATACATGCGACTT
CGACAACTTAAACGGCTTCAGGCAAATATGGGTGCAAAGGCTTTGTATGTGGCAAATTTTGCAAAGGTTC
AAGAAAAAACCCAGATCCTCAATGAAGAATGGAAGAAGCTTCGTGTCCAACCTGTTCAGTCAATGAAGCC
TTGTACCATAGAGAGCATTTTCCCGGGATTTGCAAGCCAACATATGTTAATGAGGTCACTGAACACAGTT
GCATTGGTTCCCATCATGTATTCCTGGTCCCCTCTCCAACAGAACTTTATGGTAGAAGATGAGACGGTTT
TGTGCAATATTCCCTACATGGGAGATGAAGTGAAAGAAGAAGATGAGACTTTTATTGAGGAGCTGATCAA
TAACTATGATGGGAAAGTCCATGGTGAAGAAGAGATGATCCCTGGATCCGTTCTGATTAGTGATGCTGTT
TTTCTGGAGTTGGTCGATGCCCTGAATCAGTACTCAGATGAGGAGGAGGAAGGGCACAATGACACCTCAG
ATGGAAAGCAGGATGACAGCAAAGAAGATCTGCCAGTAACAAGAAAGAGAAAGCGACATGCTATTGAAGG
CAACAAAAAGAGTTCCAAGAAACAGTTCCCAAATGACATGATCTTCAGTGCAATTGCCTCAATGTTCCCT
GAGAATGGTGTCCCAGATGACATGAAGGAGAGGTATCGAGAACTAACAGAGATGTCAGACCCCAATGCAC
TTCCCCCTCAGTGCACACCCAACATCGATGGCCCCAATGCCAAGTCTGTGCAGCGGGAGCAATCTCTGCA
CTCCTTCCACACACTTTTTTGCCGGCGCTGCTTTAAATACGACTGCTTCCTTCACCCTTTTCATGCCACC
CCTAATGTATATAAACGCAAGAATAAAGAAATCAAGATTGAACCAGAACCATGTGGCACAGACTGCTTCC
TTTTGCTGGAAGGAGCAAAGGAGTATGCCATGCTCCACAACCCCCGCTCCAAGTGCTCTGGTCGTCGCCG
GAGAAGGCACCACATAGTCAGTGCTTCCTGCTCCAATGCCTCAGCCTCTGCTGTGGCTGAGACTAAAGAA
GGAGACAGTGACAGGGACACAGGCAATGACTGGGCCTCCAGTTCTTCAGAGGCTAACTCTCGCTGTCAGA
CTCCCACAAAACAGAAGGCTAGTCCAGCCCCACCTCAACTCTGCGTAGTGGAAGCACCCTCGGAGCCTGT
GGAATGGACTGGGGCTGAAGAATCTCTTTTTCGAGTCTTCCATGGCACCTACTTCAACAACTTCTGTTCA
ATAGCCAGGCTTCTGGGGACCAAGACGTGCAAGCAGGTCTTTCAGTTTGCAGTCAAAGAATCACTTATCC
TGAAGCTGCCAACAGATGAGCTCATGAACCCCTCACAGAAGAAGAAAAGAAAGCACAGATTGTGGGCTGC
ACACTGCAGGAAGATTCAGCTGAAGAAAGATAACTCTTCCACACAAGTGTACAACTACCAACCCTGCGAC
CACCCAGACCGCCCCTGTGACAGCACCTGCCCCTGCATCATGACTCAGAATTTCTGTGAGAAGTTCTGCC
AGTGCAACCCAGACTGTCAGAATCGTTTCCCTGGCTGTCGCTGTAAGACCCAGTGCAATACCAAGCAATG
TCCTTGCTATCTGGCAGTGCGAGAATGTGACCCTGACCTGTGTCTCACCTGTGGGGCCTCAGAGCACTGG
GACTGCAAGGTGGTTTCCTGTAAAAACTGCAGCATCCAGCGTGGACTTAAGAAGCACCTGCTGCTGGCCC
CCTCTGATGTGGCCGGATGGGGCACCTTCATAAAGGAGTCTGTGCAGAAGAACGAATTCATTTCTGAATA
CTGTGGTGAGCTCATCTCTCAGGATGAGGCTGATCGACGCGGAAAGGTCTATGACAAATACATGTCCAGC
TTCCTCTTCAACCTCAATAATGATTTTGTAGTGGATGCTACTCGGAAAGGAAACAAAATTCGATTTGCAA
ATCATTCAGTGAATCCCAACTGTTATGCCAAAGTGGTCATGGTGAATGGAGACCATCGGATTGGGATCTT
TGCCAAGAGGGCAATTCAAGCTGGCGAAGAGCTCTTCTTTGATTACAGGTACAGCCAAGCTGATGCTCTC
AAGTACGTGGGGATCGAGAGGGAGACCGACGTCCTTTAGCCCTCCCAGGCCCCACGGCAGCACTTATGGT
AGCGGCACTGTCTTGGCTTTCGTGCTCACACCACTGCTGCTCGAGTCTCCTGCACTGTGTCTCCCACACT
GAGAAACCCCCCAACCCACTCCCTCTGTAGTGAGGCCTCTGCCATGTCCAGAGGGCACAAAACTGTCTCA
ATGAGAGGGGAGACAGAGGCAGCTAGGGCTTGGTCTCCCAGGACAGAGAGTTACAGAAATGGGAGACTGT
TTCTCTGGCCTCAGAAGAAGCGAGCACAGGCTGGGGTGGATGACTTATGCGTGATTTCGTGTCGGCTCCC
CAGGCTGTGGCCTCAGGAATCAACTTAGGCAGTTCCCAACAAGCGCTAGCCTGTAATTGTAGCTTTCCAC
ATCAAGAGTCCTTATGTTATTGGGATGCAGGCAAACCTCTGTGGTCCTAAGACCTGGAGAGGACAGGCTA
AGTGAAGTGTGGTCCCTGGAGCCTACAAGTGGTCTGGGTTAGAGGCGAGCCTGGCAGGCAGCACAGACTG
AACTCAGAGGTAGACAGGTCACCTTACTACCTCCTCCCTCGTGGCAGGGCTCAAACTGAAAGAGTGTGGG
TTCTAAGTACAGGCATTCAAGGCTGGGGGAAGGAAAGCTACGCCATCCTTCCTTAGCCAGAGAGGGAGAA
CCAGCCAGATGATAGTAGTTAAACTGCTAAGCTTGGGCCCAGGAGGCTTTGAGAAAGCCTTCTCTGTGTA
CTCTGGAGATAGATGGAGAAGTGTTTTCAGATTCCTGGGAACAGACACCAGTGCTCCAGCTCCTCCAAAG
TTCTGGCTTAGCAGCTGCAGGCAAGCATTATGCTGCTATTGAAGAAGCATTAGGGGTATGCCTGGCAGGT
GTGAGCATCCTGGCTCGCTGGATTTGTGGGTGTTTTCAGGCCTTCCATTCCCCATAGAGGCAAGGCCCAA
TGGCCAGTGTTGCTTATCGCTTCAGGGTAGGTGGGCACAGGCTTGGACTAGAGAGGAGAAAGATTGGTGT
AATCTGCTTTCCTGTCTGTAGTGCCTGCTGTTTGGAAAGGGTGAGTTAGAATATGTTCCAAGGTTGGTGA
GGGGCTAAATTGCACGCGTTTAGGCTGGCACCCCGTGTGCAGGGCACACTGGCAGAGGGTATCTGAAGTG
GGAGAAGAAGCAGGTAGACCACCTGTCCCAGGCTGTGGTGCCACCCTCTCTGGCATTCATGCAGAGCAAA
GCACTTTAACCATTTCTTTTAAAAGGTCTATAGATTGGGGTAGAGTTTGGCCTAAGGTCTCTAGGGTCCC
TGCCTAAATCCCACTCCTGAGGGAGGGGGAAGAAGAGAGGGTGGGAGATTCTCCTCCAGTCCTGTCTCAT
CTCCTGGGAGAGGCAGACGAGTGAGTTTCACACAGAAGAATTTCATGTGAATGGGGCCAGCAAGAGCTGC
CCTGTGTCCATGGTGGGTGTGCCGGGCTGGCTGGGAACAAGGAGCAGTATGTTGAGTAGAAAGGGTGTGG
GCGGGTATAGATTGGCCTGGGAGTGTTACAGTAGGGAGCAGGCTTCTCCCTTCTTTCTGGGACTCAGAGC
CCCGCTTCTTCCCACTCCACTTGTTGTCCCATGAAGGAAGAAGTGGGGTTCCTCCTGACCCAGCTGCCTC
TTACGGTTTGGTATGGGACATGCACACACACTCACATGCTCTCACTCACCACACTGGAGGGCACACACGT
ACCCCGCACCCAGCAACTCCTGACAGAAAGCTCCTCCCACCCAAATGGGCCAGGCCCCAGCATGATCCTG
AAATCTGCATCCGCCGTGGTTTGTATTCATTGTGCATATCAGGGATACCCTCAAGCTGGACTGTGGGTTC
CAAATTACTCATAGAGGAGAAAACCAGAGAAAGATGAAGAGGAGGAGTTAGGTCTATTTGAAATGCCAGG
GGCTCGCTGTGAGGAATAGGTGAAAAAAAACTTTTCACCAGCCTTTGAGAGACTAGACTGACCCCACCCT
TCCTTCAGTGAGCAGAATCACTGTGGTCAGTCTCCTGTCCCAGCTTCAGTTCATGAATACTCCTGTTCCT
CCAGTTTCCCATCCTTTGTCCCTGCTGTCCCCCACTTTTAAAGATGGGTCTCAACCCCTCCCCACCACGT
CATGATGGATGGGGCAAGGTGGTGGGGACTAGGGGAGCCTGGTATACATGCGGCTTCATTGCCAATAAAT
TTCATGCACTTTAAAGTCCTGTGGCTTGTGACCTCTTAATAAAGTGTTAGAATCCA
NM_001203247.2 Homo sapiens enhancer of zeste 2 polycomb repressive
complex 2 subunit (EZH2), transcript variant 3, mRNA
(SEQ ID NO: 72)
GTTTGGCGCTCGGTCCGGTCGCGTCCGACACCCGGTGGGACTCAGAAGGCAGTGGAGCCCCGGCGGCGGC
GGCGGCGGCGCGCGGGGGCGACGCGCGGGAACAACGCGAGTCGGCGCGCGGGACGAAGAATAATCATGGG
CCAGACTGGGAAGAAATCTGAGAAGGGACCAGTTTGTTGGCGGAAGCGTGTAAAATCAGAGTACATGCGA
CTGAGACAGCTCAAGAGGTTCAGACGAGCTGATGAAGTAAAGAGTATGTTTAGTTCCAATCGTCAGAAAA
TTTTGGAAAGAACGGAAATCTTAAACCAAGAATGGAAACAGCGAAGGATACAGCCTGTGCACATCCTGAC
TTCTGTGAGCTCATTGCGCGGGACTAGGGAGTGTTCGGTGACCAGTGACTTGGATTTTCCAACACAAGTC
ATCCCATTAAAGACTCTGAATGCAGTTGCTTCAGTACCCATAATGTATTCTTGGTCTCCCCTACAGCAGA
ATTTTATGGTGGAAGATGAAACTGTTTTACATAACATTCCTTATATGGGAGATGAAGTTTTAGATCAGGA
TGGTACTTTCATTGAAGAACTAATAAAAAATTATGATGGGAAAGTACACGGGGATAGAGAATGTGGGTTT
ATAAATGATGAAATTTTTGTGGAGTTGGTGAATGCCCTTGGTCAATATAATGATGATGACGATGATGATG
ATGGAGACGATCCTGAAGAAAGAGAAGAAAAGCAGAAAGATCTGGAGGATCACCGAGATGATAAAGAAAG
CCGCCCACCTCGGAAATTTCCTTCTGATAAAATTTTTGAAGCCATTTCCTCAATGTTTCCAGATAAGGGC
ACAGCAGAAGAACTAAAGGAAAAATATAAAGAACTCACCGAACAGCAGCTCCCAGGCGCACTTCCTCCTG
AATGTACCCCCAACATAGATGGACCAAATGCTAAATCTGTTCAGAGAGAGCAAAGCTTACACTCCTTTCA
TACGCTTTTCTGTAGGCGATGTTTTAAATATGACTGCTTCCTACATCCTTTTCATGCAACACCCAACACT
TATAAGCGGAAGAACACAGAAACAGCTCTAGACAACAAACCTTGTGGACCACAGTGTTACCAGCATTTGG
AGGGAGCAAAGGAGTTTGCTGCTGCTCTCACCGCTGAGCGGATAAAGACCCCACCAAAACGTCCAGGAGG
CCGCAGAAGAGGACGGCTTCCCAATAACAGTAGCAGGCCCAGCACCCCCACCATTAATGTGCTGGAATCA
AAGGATACAGACAGTGATAGGGAAGCAGGGACTGAAACGGGGGGAGAGAACAATGATAAAGAAGAAGAAG
AGAAGAAAGATGAAACTTCGAGCTCCTCTGAAGCAAATTCTCGGTGTCAAACACCAATAAAGATGAAGCC
AAATATTGAACCTCCTGAGAATGTGGAGTGGAGTGGTGCTGAAGCCTCAATGTTTAGAGTCCTCATTGGC
ACTTACTATGACAATTTCTGTGCCATTGCTAGGTTAATTGGGACCAAAACATGTAGACAGGTGTATGAGT
TTAGAGTCAAAGAATCTAGCATCATAGCTCCAGCTCCCGCTGAGGATGTGGATACTCCTCCAAGGAAAAA
GAAGAGGAAACACCGGTTGTGGGCTGCACACTGCAGAAAGATACAGCTGAAAAAGGACGGCTCCTCTAAC
CATGTTTACAACTATCAACCCTGTGATCATCCACGGCAGCCTTGTGACAGTTCGTGCCCTTGTGTGATAG
CACAAAATTTTTGTGAAAAGTTTTGTCAATGTAGTTCAGAGTGTCAAAACCGCTTTCCGGGATGCCGCTG
CAAAGCACAGTGCAACACCAAGCAGTGCCCGTGCTACCTGGCTGTCCGAGAGTGTGACCCTGACCTCTGT
CTTACTTGTGGAGCCGCTGACCATTGGGACAGTAAAAATGTGTCCTGCAAGAACTGCAGTATTCAGCGGG
GCTCCAAAAAGCATCTATTGCTGGCACCATCTGACGTGGCAGGCTGGGGGATTTTTATCAAAGATCCTGT
GCAGAAAAATGAATTCATCTCAGAATACTGTGGAGAGATTATTTCTCAAGATGAAGCTGACAGAAGAGGG
AAAGTGTATGATAAATACATGTGCAGCTTTCTGTTCAACTTGAACAATGATTTTGTGGTGGATGCAACCC
GCAAGGGTAACAAAATTCGTTTTGCAAATCATTCGGTAAATCCAAACTGCTATGCAAAAGTTATGATGGT
TAACGGTGATCACAGGATAGGTATTTTTGCCAAGAGAGCCATCCAGACTGGCGAAGAGCTGTTTTTTGAT
TACAGATACAGCCAGGCTGATGCCCTGAAGTATGTCGGCATCGAAAGAGAAATGGAAATCCCTTGACATC
TGCTACCTCCTCCCCCCTCCTCTGAAACAGCTGCCTTAGCTTCAGGAACCTCGAGTACTGTGGGCAATTT
AGAAAAAGAACATGCAGTTTGAAATTCTGAATTTGCAAAGTACTGTAAGAATAATTTATAGTAATGAGTT
TAAAAATCAACTTTTTATTGCCTTCTCACCAGCTGCAAAGTGTTTTGTACCAGTGAATTTTTGCAATAAT
GCAGTATGGTACATTTTTCAACTTTGAATAAAGAATACTTGAACTTGTC
NM_001203248.2 Homo sapiens enhancer of zeste 2 polycomb repressive
complex 2 subunit (EZH2), transcript variant 4, mRNA
(SEQ ID NO: 73)
GTTTGGCGCTCGGTCCGGTCGCGTCCGACACCCGGTGGGACTCAGAAGGCAGTGGAGCCCCGGCGGCGGC
GGCGGCGGCGCGCGGGGGCGACGCGCGGGAACAACGCGAGTCGGCGCGCGGGACGAAGAATAATCATGGG
CCAGACTGGGAAGAAATCTGAGAAGGGACCAGTTTGTTGGCGGAAGCGTGTAAAATCAGAGTACATGCGA
CTGAGACAGCTCAAGAGGTTCAGACGAGCTGATGAAGTAAAGAGTATGTTTAGTTCCAATCGTCAGAAAA
TTTTGGAAAGAACGGAAATCTTAAACCAAGAATGGAAACAGCGAAGGATACAGCCTGTGCACATCCTGAC
TTCTTGTTCGGTGACCAGTGACTTGGATTTTCCAACACAAGTCATCCCATTAAAGACTCTGAATGCAGTT
GCTTCAGTACCCATAATGTATTCTTGGTCTCCCCTACAGCAGAATTTTATGGTGGAAGATGAAACTGTTT
TACATAACATTCCTTATATGGGAGATGAAGTTTTAGATCAGGATGGTACTTTCATTGAAGAACTAATAAA
AAATTATGATGGGAAAGTACACGGGGATAGAGAATGTGGGTTTATAAATGATGAAATTTTTGTGGAGTTG
GTGAATGCCCTTGGTCAATATAATGATGATGACGATGATGATGATGGAGACGATCCTGAAGAAAGAGAAG
AAAAGCAGAAAGATCTGGAGGATCACCGAGATGATAAAGAAAGCCGCCCACCTCGGAAATTTCCTTCTGA
TAAAATTTTTGAAGCCATTTCCTCAATGTTTCCAGATAAGGGCACAGCAGAAGAACTAAAGGAAAAATAT
AAAGAACTCACCGAACAGCAGCTCCCAGGCGCACTTCCTCCTGAATGTACCCCCAACATAGATGGACCAA
ATGCTAAATCTGTTCAGAGAGAGCAAAGCTTACACTCCTTTCATACGCTTTTCTGTAGGCGATGTTTTAA
ATATGACTGCTTCCTACATCCTTTTCATGCAACACCCAACACTTATAAGCGGAAGAACACAGAAACAGCT
CTAGACAACAAACCTTGTGGACCACAGTGTTACCAGCATTTGGAGGGAGCAAAGGAGTTTGCTGCTGCTC
TCACCGCTGAGCGGATAAAGACCCCACCAAAACGTCCAGGAGGCCGCAGAAGAGGACGGCTTCCCAATAA
CAGTAGCAGGCCCAGCACCCCCACCATTAATGTGCTGGAATCAAAGGATACAGACAGTGATAGGGAAGCA
GGGACTGAAACGGGGGGAGAGAACAATGATAAAGAAGAAGAAGAGAAGAAAGATGAAACTTCGAGCTCCT
CTGAAGCAAATTCTCGGTGTCAAACACCAATAAAGATGAAGCCAAATATTGAACCTCCTGAGAATGTGGA
GTGGAGTGGTGCTGAAGCCTCAATGTTTAGAGTCCTCATTGGCACTTACTATGACAATTTCTGTGCCATT
GCTAGGTTAATTGGGACCAAAACATGTAGACAGGTGTATGAGTTTAGAGTCAAAGAATCTAGCATCATAG
CTCCAGCTCCCGCTGAGGATGTGGATACTCCTCCAAGGAAAAAGAAGAGGAAACACCGGTTGTGGGCTGC
ACACTGCAGAAAGATACAGCTGAAAAAGGACGGCTCCTCTAACCATGTTTACAACTATCAACCCTGTGAT
CATCCACGGCAGCCTTGTGACAGTTCGTGCCCTTGTGTGATAGCACAAAATTTTTGTGAAAAGTTTTGTC
AATGTAGTTCAGAGTGTCAAAACCGCTTTCCGGGATGCCGCTGCAAAGCACAGTGCAACACCAAGCAGTG
CCCGTGCTACCTGGCTGTCCGAGAGTGTGACCCTGACCTCTGTCTTACTTGTGGAGCCGCTGACCATTGG
GACAGTAAAAATGTGTCCTGCAAGAACTGCAGTATTCAGCGGGGCTCCAAAAAGCATCTATTGCTGGCAC
CATCTGACGTGGCAGGCTGGGGGATTTTTATCAAAGATCCTGTGCAGAAAAATGAATTCATCTCAGAATA
CTGTGGAGAGATTATTTCTCAAGATGAAGCTGACAGAAGAGGGAAAGTGTATGATAAATACATGTGCAGC
TTTCTGTTCAACTTGAACAATGATTTTGTGGTGGATGCAACCCGCAAGGGTAACAAAATTCGTTTTGCAA
ATCATTCGGTAAATCCAAACTGCTATGCAAAAGTTATGATGGTTAACGGTGATCACAGGATAGGTATTTT
TGCCAAGAGAGCCATCCAGACTGGCGAAGAGCTGTTTTTTGATTACAGATACAGCCAGGCTGATGCCCTG
AAGTATGTCGGCATCGAAAGAGAAATGGAAATCCCTTGACATCTGCTACCTCCTCCCCCCTCCTCTGAAA
CAGCTGCCTTAGCTTCAGGAACCTCGAGTACTGTGGGCAATTTAGAAAAAGAACATGCAGTTTGAAATTC
TGAATTTGCAAAGTACTGTAAGAATAATTTATAGTAATGAGTTTAAAAATCAACTTTTTATTGCCTTCTC
ACCAGCTGCAAAGTGTTTTGTACCAGTGAATTTTTGCAATAATGCAGTATGGTACATTTTTCAACTTTGA
ATAAAGAATACTTGAACTTGTC
NM_001203249.2 Homo sapiens enhancer of zeste 2 polycomb repressive
complex 2 subunit (EZH2), transcript variant 5, mRNA
(SEQ ID NO: 74)
GGAGTAGCTTCGCCTCTGACGTTTCCCCACGACGCACCCCGAAATCCCCCTGAGCTCCGGCGGTCGCGGG
CTGCCCTCGCCGCCTGGTCTGGCTTTATGCTAAGTTTGAGGGAAGAGTCGAGCTGCTCTGCTCTCTATTG
ATTGTGTTTCTGGAGGGCGTCCTGTTGAATTCCCACTTCATTGTGTACATCCCCTTCCGTTCCCCCCAAA
AATCTGTGCCACAGGGTTACTTTTTGAAAGCGGGAGGAATCGAGAAGCACGATCTTTTGGAAAACTTGGT
GAACGCCTAAATAATCATGGGCCAGACTGGGAAGAAATCTGAGAAGGGACCAGTTTGTTGGCGGAAGCGT
GTAAAATCAGAGTACATGCGACTGAGACAGCTCAAGAGGTTCAGACGAGCTGATGAAGTAAAGAGTATGT
TTAGTTCCAATCGTCAGAAAATTTTGGAAAGAACGGAAATCTTAAACCAAGAATGGAAACAGCGAAGGAT
ACAGCCTGTGCACATCCTGACTTCTTGTTCGGTGACCAGTGACTTGGATTTTCCAACACAAGTCATCCCA
TTAAAGACTCTGAATGCAGTTGCTTCAGTACCCATAATGTATTCTTGGTCTCCCCTACAGCAGAATTTTA
TGGTGGAAGATGAAACTGTTTTACATAACATTCCTTATATGGGAGATGAAGTTTTAGATCAGGATGGTAC
TTTCATTGAAGAACTAATAAAAAATTATGATGGGAAAGTACACGGGGATAGAGAATGTGGGTTTATAAAT
GATGAAATTTTTGTGGAGTTGGTGAATGCCCTTGGTCAATATAATGATGATGACGATGATGATGATGGAG
ACGATCCTGAAGAAAGAGAAGAAAAGCAGAAAGATCTGGAGGATCACCGAGATGATAAAGAAAGCCGCCC
ACCTCGGAAATTTCCTTCTGATAAAATTTTTGAAGCCATTTCCTCAATGTTTCCAGATAAGGGCACAGCA
GAAGAACTAAAGGAAAAATATAAAGAACTCACCGAACAGCAGCTCCCAGGCGCACTTCCTCCTGAATGTA
CCCCCAACATAGATGGACCAAATGCTAAATCTGTTCAGAGAGAGCAAAGCTTACACTCCTTTCATACGCT
TTTCTGTAGGCGATGTTTTAAATATGACTGCTTCCTACATCCTTTTCATGCAACACCCAACACTTATAAG
CGGAAGAACACAGAAACAGCTCTAGACAACAAACCTTGTGGACCACAGTGTTACCAGCATTTGGAGGGAG
CAAAGGAGTTTGCTGCTGCTCTCACCGCTGAGCGGATAAAGACCCCACCAAAACGTCCAGGAGGCCGCAG
AAGAGGACGGCTTCCCAATAACAGTAGCAGGCCCAGCACCCCCACCATTAATGTGCTGGAATCAAAGGAT
ACAGACAGTGATAGGGAAGCAGGGACTGAAACGGGGGGAGAGAACAATGATAAAGAAGAAGAAGAGAAGA
AAGATGAAACTTCGAGCTCCTCTGAAGCAAATTCTCGGTGTCAAACACCAATAAAGATGAAGCCAAATAT
TGAACCTCCTGAGAATGTGGAGTGGAGTGGTGCTGAAGCCTCAATGTTTAGAGTCCTCATTGGCACTTAC
TATGACAATTTCTGTGCCATTGCTAGGTTAATTGGGACCAAAACATGTAGACAGGTGTATGAGTTTAGAG
TCAAAGAATCTAGCATCATAGCTCCAGCTCCCGCTGAGGATGTGGATACTCCTCCAAGGAAAAAGAAGAG
GAAACACCGGTTGTGGGCTGCACACTGCAGAAAGATACAGCTGAAAAAGGGTCAAAACCGCTTTCCGGGA
TGCCGCTGCAAAGCACAGTGCAACACCAAGCAGTGCCCGTGCTACCTGGCTGTCCGAGAGTGTGACCCTG
ACCTCTGTCTTACTTGTGGAGCCGCTGACCATTGGGACAGTAAAAATGTGTCCTGCAAGAACTGCAGTAT
TCAGCGGGGCTCCAAAAAGCATCTATTGCTGGCACCATCTGACGTGGCAGGCTGGGGGATTTTTATCAAA
GATCCTGTGCAGAAAAATGAATTCATCTCAGAATACTGTGGAGAGATTATTTCTCAAGATGAAGCTGACA
GAAGAGGGAAAGTGTATGATAAATACATGTGCAGCTTTCTGTTCAACTTGAACAATGATTTTGTGGTGGA
TGCAACCCGCAAGGGTAACAAAATTCGTTTTGCAAATCATTCGGTAAATCCAAACTGCTATGCAAAAGTT
ATGATGGTTAACGGTGATCACAGGATAGGTATTTTTGCCAAGAGAGCCATCCAGACTGGCGAAGAGCTGT
TTTTTGATTACAGATACAGCCAGGCTGATGCCCTGAAGTATGTCGGCATCGAAAGAGAAATGGAAATCCC
TTGACATCTGCTACCTCCTCCCCCCTCCTCTGAAACAGCTGCCTTAGCTTCAGGAACCTCGAGTACTGTG
GGCAATTTAGAAAAAGAACATGCAGTTTGAAATTCTGAATTTGCAAAGTACTGTAAGAATAATTTATAGT
AATGAGTTTAAAAATCAACTTTTTATTGCCTTCTCACCAGCTGCAAAGTGTTTTGTACCAGTGAATTTTT
GCAATAATGCAGTATGGTACATTTTTCAACTTTGAATAAAGAATACTTGAACTTGTC
NM_004456.5 Homo sapiens enhancer of zeste 2 polycomb repressive complex 2
subunit (EZH2), transcript variant 1, mRNA
(SEQ ID NO: 75)
GTTTGGCGCTCGGTCCGGTCGCGTCCGACACCCGGTGGGACTCAGAAGGCAGTGGAGCCCCGGCGGCGGC
GGCGGCGGCGCGCGGGGGCGACGCGCGGGAACAACGCGAGTCGGCGCGCGGGACGAAGAATAATCATGGG
CCAGACTGGGAAGAAATCTGAGAAGGGACCAGTTTGTTGGCGGAAGCGTGTAAAATCAGAGTACATGCGA
CTGAGACAGCTCAAGAGGTTCAGACGAGCTGATGAAGTAAAGAGTATGTTTAGTTCCAATCGTCAGAAAA
TTTTGGAAAGAACGGAAATCTTAAACCAAGAATGGAAACAGCGAAGGATACAGCCTGTGCACATCCTGAC
TTCTGTGAGCTCATTGCGCGGGACTAGGGAGTGTTCGGTGACCAGTGACTTGGATTTTCCAACACAAGTC
ATCCCATTAAAGACTCTGAATGCAGTTGCTTCAGTACCCATAATGTATTCTTGGTCTCCCCTACAGCAGA
ATTTTATGGTGGAAGATGAAACTGTTTTACATAACATTCCTTATATGGGAGATGAAGTTTTAGATCAGGA
TGGTACTTTCATTGAAGAACTAATAAAAAATTATGATGGGAAAGTACACGGGGATAGAGAATGTGGGTTT
ATAAATGATGAAATTTTTGTGGAGTTGGTGAATGCCCTTGGTCAATATAATGATGATGACGATGATGATG
ATGGAGACGATCCTGAAGAAAGAGAAGAAAAGCAGAAAGATCTGGAGGATCACCGAGATGATAAAGAAAG
CCGCCCACCTCGGAAATTTCCTTCTGATAAAATTTTTGAAGCCATTTCCTCAATGTTTCCAGATAAGGGC
ACAGCAGAAGAACTAAAGGAAAAATATAAAGAACTCACCGAACAGCAGCTCCCAGGCGCACTTCCTCCTG
AATGTACCCCCAACATAGATGGACCAAATGCTAAATCTGTTCAGAGAGAGCAAAGCTTACACTCCTTTCA
TACGCTTTTCTGTAGGCGATGTTTTAAATATGACTGCTTCCTACATCGTAAGTGCAATTATTCTTTTCAT
GCAACACCCAACACTTATAAGCGGAAGAACACAGAAACAGCTCTAGACAACAAACCTTGTGGACCACAGT
GTTACCAGCATTTGGAGGGAGCAAAGGAGTTTGCTGCTGCTCTCACCGCTGAGCGGATAAAGACCCCACC
AAAACGTCCAGGAGGCCGCAGAAGAGGACGGCTTCCCAATAACAGTAGCAGGCCCAGCACCCCCACCATT
AATGTGCTGGAATCAAAGGATACAGACAGTGATAGGGAAGCAGGGACTGAAACGGGGGGAGAGAACAATG
ATAAAGAAGAAGAAGAGAAGAAAGATGAAACTTCGAGCTCCTCTGAAGCAAATTCTCGGTGTCAAACACC
AATAAAGATGAAGCCAAATATTGAACCTCCTGAGAATGTGGAGTGGAGTGGTGCTGAAGCCTCAATGTTT
AGAGTCCTCATTGGCACTTACTATGACAATTTCTGTGCCATTGCTAGGTTAATTGGGACCAAAACATGTA
GACAGGTGTATGAGTTTAGAGTCAAAGAATCTAGCATCATAGCTCCAGCTCCCGCTGAGGATGTGGATAC
TCCTCCAAGGAAAAAGAAGAGGAAACACCGGTTGTGGGCTGCACACTGCAGAAAGATACAGCTGAAAAAG
GACGGCTCCTCTAACCATGTTTACAACTATCAACCCTGTGATCATCCACGGCAGCCTTGTGACAGTTCGT
GCCCTTGTGTGATAGCACAAAATTTTTGTGAAAAGTTTTGTCAATGTAGTTCAGAGTGTCAAAACCGCTT
TCCGGGATGCCGCTGCAAAGCACAGTGCAACACCAAGCAGTGCCCGTGCTACCTGGCTGTCCGAGAGTGT
GACCCTGACCTCTGTCTTACTTGTGGAGCCGCTGACCATTGGGACAGTAAAAATGTGTCCTGCAAGAACT
GCAGTATTCAGCGGGGCTCCAAAAAGCATCTATTGCTGGCACCATCTGACGTGGCAGGCTGGGGGATTTT
TATCAAAGATCCTGTGCAGAAAAATGAATTCATCTCAGAATACTGTGGAGAGATTATTTCTCAAGATGAA
GCTGACAGAAGAGGGAAAGTGTATGATAAATACATGTGCAGCTTTCTGTTCAACTTGAACAATGATTTTG
TGGTGGATGCAACCCGCAAGGGTAACAAAATTCGTTTTGCAAATCATTCGGTAAATCCAAACTGCTATGC
AAAAGTTATGATGGTTAACGGTGATCACAGGATAGGTATTTTTGCCAAGAGAGCCATCCAGACTGGCGAA
GAGCTGTTTTTTGATTACAGATACAGCCAGGCTGATGCCCTGAAGTATGTCGGCATCGAAAGAGAAATGG
AAATCCCTTGACATCTGCTACCTCCTCCCCCCTCCTCTGAAACAGCTGCCTTAGCTTCAGGAACCTCGAG
TACTGTGGGCAATTTAGAAAAAGAACATGCAGTTTGAAATTCTGAATTTGCAAAGTACTGTAAGAATAAT
TTATAGTAATGAGTTTAAAAATCAACTTTTTATTGCCTTCTCACCAGCTGCAAAGTGTTTTGTACCAGTG
AATTTTTGCAATAATGCAGTATGGTACATTTTTCAACTTTGAATAAAGAATACTTGAACTTGTC
NM_152998.3 Homo sapiens enhancer of zeste 2 polycomb repressive complex 2
subunit (EZH2), transcript variant 2, mRNA
(SEQ ID NO: 76)
GTTTGGCGCTCGGTCCGGTCGCGTCCGACACCCGGTGGGACTCAGAAGGCAGTGGAGCCCCGGCGGCGGC
GGCGGCGGCGCGCGGGGGCGACGCGCGGGAACAACGCGAGTCGGCGCGCGGGACGAAGAATAATCATGGG
CCAGACTGGGAAGAAATCTGAGAAGGGACCAGTTTGTTGGCGGAAGCGTGTAAAATCAGAGTACATGCGA
CTGAGACAGCTCAAGAGGTTCAGACGAGCTGATGAAGTAAAGAGTATGTTTAGTTCCAATCGTCAGAAAA
TTTTGGAAAGAACGGAAATCTTAAACCAAGAATGGAAACAGCGAAGGATACAGCCTGTGCACATCCTGAC
TTCTGTGAGCTCATTGCGCGGGACTAGGGAGGTGGAAGATGAAACTGTTTTACATAACATTCCTTATATG
GGAGATGAAGTTTTAGATCAGGATGGTACTTTCATTGAAGAACTAATAAAAAATTATGATGGGAAAGTAC
ACGGGGATAGAGAATGTGGGTTTATAAATGATGAAATTTTTGTGGAGTTGGTGAATGCCCTTGGTCAATA
TAATGATGATGACGATGATGATGATGGAGACGATCCTGAAGAAAGAGAAGAAAAGCAGAAAGATCTGGAG
GATCACCGAGATGATAAAGAAAGCCGCCCACCTCGGAAATTTCCTTCTGATAAAATTTTTGAAGCCATTT
CCTCAATGTTTCCAGATAAGGGCACAGCAGAAGAACTAAAGGAAAAATATAAAGAACTCACCGAACAGCA
GCTCCCAGGCGCACTTCCTCCTGAATGTACCCCCAACATAGATGGACCAAATGCTAAATCTGTTCAGAGA
GAGCAAAGCTTACACTCCTTTCATACGCTTTTCTGTAGGCGATGTTTTAAATATGACTGCTTCCTACATC
CTTTTCATGCAACACCCAACACTTATAAGCGGAAGAACACAGAAACAGCTCTAGACAACAAACCTTGTGG
ACCACAGTGTTACCAGCATTTGGAGGGAGCAAAGGAGTTTGCTGCTGCTCTCACCGCTGAGCGGATAAAG
ACCCCACCAAAACGTCCAGGAGGCCGCAGAAGAGGACGGCTTCCCAATAACAGTAGCAGGCCCAGCACCC
CCACCATTAATGTGCTGGAATCAAAGGATACAGACAGTGATAGGGAAGCAGGGACTGAAACGGGGGGAGA
GAACAATGATAAAGAAGAAGAAGAGAAGAAAGATGAAACTTCGAGCTCCTCTGAAGCAAATTCTCGGTGT
CAAACACCAATAAAGATGAAGCCAAATATTGAACCTCCTGAGAATGTGGAGTGGAGTGGTGCTGAAGCCT
CAATGTTTAGAGTCCTCATTGGCACTTACTATGACAATTTCTGTGCCATTGCTAGGTTAATTGGGACCAA
AACATGTAGACAGGTGTATGAGTTTAGAGTCAAAGAATCTAGCATCATAGCTCCAGCTCCCGCTGAGGAT
GTGGATACTCCTCCAAGGAAAAAGAAGAGGAAACACCGGTTGTGGGCTGCACACTGCAGAAAGATACAGC
TGAAAAAGGACGGCTCCTCTAACCATGTTTACAACTATCAACCCTGTGATCATCCACGGCAGCCTTGTGA
CAGTTCGTGCCCTTGTGTGATAGCACAAAATTTTTGTGAAAAGTTTTGTCAATGTAGTTCAGAGTGTCAA
AACCGCTTTCCGGGATGCCGCTGCAAAGCACAGTGCAACACCAAGCAGTGCCCGTGCTACCTGGCTGTCC
GAGAGTGTGACCCTGACCTCTGTCTTACTTGTGGAGCCGCTGACCATTGGGACAGTAAAAATGTGTCCTG
CAAGAACTGCAGTATTCAGCGGGGCTCCAAAAAGCATCTATTGCTGGCACCATCTGACGTGGCAGGCTGG
GGGATTTTTATCAAAGATCCTGTGCAGAAAAATGAATTCATCTCAGAATACTGTGGAGAGATTATTTCTC
AAGATGAAGCTGACAGAAGAGGGAAAGTGTATGATAAATACATGTGCAGCTTTCTGTTCAACTTGAACAA
TGATTTTGTGGTGGATGCAACCCGCAAGGGTAACAAAATTCGTTTTGCAAATCATTCGGTAAATCCAAAC
TGCTATGCAAAAGTTATGATGGTTAACGGTGATCACAGGATAGGTATTTTTGCCAAGAGAGCCATCCAGA
CTGGCGAAGAGCTGTTTTTTGATTACAGATACAGCCAGGCTGATGCCCTGAAGTATGTCGGCATCGAAAG
AGAAATGGAAATCCCTTGACATCTGCTACCTCCTCCCCCCTCCTCTGAAACAGCTGCCTTAGCTTCAGGA
ACCTCGAGTACTGTGGGCAATTTAGAAAAAGAACATGCAGTTTGAAATTCTGAATTTGCAAAGTACTGTA
AGAATAATTTATAGTAATGAGTTTAAAAATCAACTTTTTATTGCCTTCTCACCAGCTGCAAAGTGTTTTG
TACCAGTGAATTTTTGCAATAATGCAGTATGGTACATTTTTCAACTTTGAATAAAGAATACTTGAACTTG
TC

Primers or probes can be designed so that they hybridize under stringent conditions to mutant nucleotide sequences of SUZ12, EED, or EZH1/2, but not to the respective wild-type nucleotide sequences. Primers or probes can also be prepared that are complementary and specific for the wild-type nucleotide sequence of SUZ12, EED, or EZH1/2, but not to any of the corresponding mutant nucleotide sequences. In some embodiments, the mutant nucleotide sequences of SUZ12, EED, or EZH1/2 may be a frameshift mutation, a missense mutation, a deletion, an insertion, a nonsense mutation, an inversion, or a translocation, that results in the loss of expression and/or activity of SUZ12, EED, or EZH1/2 (i.e., loss of function mutations).

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

Pharmaceutical Compositions Including Immune Checkpoint Inhibitors and/or Inactivated MVA or MVA ΔE3L

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Modes of Administration and Effective Dosages

Any method known to those in the art for contacting a cell, organ or tissue with one or more immune checkpoint inhibitors and/or inactivated MVA or MVA ΔE3L disclosed herein may be employed. Suitable methods include in vitro, ex vivo, or in vivo methods. In vivo methods typically include the administration of one or more immune checkpoint inhibitors and/or inactivated MVA or MVA ΔE3L to a mammal, suitably a human. When used in vivo for therapy, the one or more immune checkpoint inhibitors and/or inactivated MVA or MVA ΔE3L described herein are administered to the subject in effective amounts (i.e., amounts that have desired therapeutic effect). The dose and dosage regimen will depend upon the degree of the disease state of the subject, the characteristics of the particular immune checkpoint inhibitor or inactivated MVA or MVA ΔE3L used, e.g., its therapeutic index, and the subject's history.

The effective amount may be determined during pre-clinical trials and clinical trials by methods familiar to physicians and clinicians. An effective amount of one or more immune checkpoint inhibitors and/or inactivated MVA or MVA ΔE3L useful in the methods may be administered to a mammal in need thereof by any of a number of well-known methods for administering pharmaceutical compounds. The immune checkpoint inhibitor or inactivated MVA or MVA ΔE3L may be administered systemically or locally.

The one or more immune checkpoint inhibitors and/or inactivated MVA or MVA ΔE3L described herein can be incorporated into pharmaceutical compositions for administration, singly or in combination, to a subject for the treatment or prevention of a disease or condition described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more immune checkpoint inhibitors and/or inactivated MVA or MVA ΔE3L disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more immune checkpoint inhibitors and/or inactivated MVA or MVA ΔE3L disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more immune checkpoint inhibitor/inactivated MVA or MVA ΔE3L concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more immune checkpoint inhibitors and/or inactivated MVA or MVA ΔE3L may be defined as a concentration of the agent at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Therapeutic Methods of the Present Technology

In one aspect, the present disclosure provides a method for selecting a cancer patient for treatment with an immune checkpoint inhibitor comprising (a) detecting the presence of at least one mutation that results in reduced expression or activity of Polycomb Repressive Complex 2 (PRC2) in a biological sample obtained from the cancer patient; and (b) administering to the cancer patient an effective amount of inactivated modified vaccinia virus Ankara (MVA) or MVA ΔE3L and an effective amount of an immune checkpoint inhibitor. The at least one mutation may comprise a mutation in SUZ12, EED, and/or EZH1/2. In some embodiments, the at least one mutation is a nonsense mutation, a missense mutation, a deletion, an inversion or a frameshift mutation. Additionally or alternatively, in some embodiments, the at least one mutation is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

In one aspect, the present disclosure provides a method for treating cancer in a patient in need thereof comprising administering to the patient an effective amount of inactivated modified vaccinia virus Ankara (MVA) or MVA ΔE3L and an effective amount of an immune checkpoint inhibitor, wherein mRNA and/or polypeptide expression and/or activity levels of PRC2 in a biological sample obtained from the patient are reduced compared to a control sample obtained from a healthy subject or a predetermined threshold. In certain embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). In other embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any and all embodiments of the methods disclosed herein, the biological sample obtained from the cancer patient comprises biopsied tumor tissue, whole blood, plasma, or serum.

In another aspect, the present disclosure provides a method for sensitizing PRC2 mutant tumors to treatment with an immune checkpoint inhibitor in a patient in need thereof comprising administering to the patient an effective amount of inactivated modified vaccinia virus Ankara (MVA) or MVA ΔE3L separately, sequentially or simultaneously with the immune checkpoint inhibitor.

In any of the preceding embodiments of the methods disclosed herein, inactivation of MVA or MVA ΔE3L occurs via heat-induced inactivation or UV radiation-induced inactivation. Examples of immune checkpoint inhibitors include, but are not limited to one or more of a PD-1/PD-L1 inhibitor, a CTLA-4 inhibitor, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, and BMS-986189.

In any and all embodiments of the methods disclosed herein, the patient suffers from or is diagnosed with malignant peripheral nerve sheath tumor (MPNST), melanoma, a myeloid disorder, T-cell acute lymphocytic leukemia (ALL), early T-cell precursor ALL, pediatric glioma, or invasive breast cancer.

Additionally or alternatively, in some embodiments of the methods disclosed herein, the immune checkpoint inhibitor can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), simultaneously with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an inactivated MVA or MVA ΔE3L to a patient with cancer (e.g., malignant peripheral nerve sheath tumor or melanoma).

In some embodiments, the immune checkpoint inhibitor and inactivated MVA or MVA ΔE3L are administered to a patient, for example, a mammal, such as a human, in a sequence and within a time interval such that the inhibitor that is administered first acts together with the inhibitor that is administered second to provide greater benefit than if each inhibitor were administered alone. For example, the immune checkpoint inhibitor and inactivated MVA or MVA ΔE3L can be administered at the same time or sequentially in any order at different points in time; however, if not administered at the same time, the immune checkpoint inhibitor and inactivated MVA or MVA ΔE3L are administered sufficiently close in time so as to provide the desired therapeutic or prophylactic effect of the combination of the two inhibitors. In one embodiment, the immune checkpoint inhibitor and inactivated MVA or MVA ΔE3L exert their effects at times which overlap. In some embodiments, the immune checkpoint inhibitor and inactivated MVA or MVA ΔE3L each are administered as separate dosage forms, in any appropriate form and by any suitable route. In other embodiments, the immune checkpoint inhibitor and inactivated MVA or MVA ΔE3L are administered simultaneously in a single dosage form.

It will be appreciated that the frequency with which any of these therapeutic agents can be administered can be once or more than once over a period of about 2 days, about 3 days, about 4 days, about 5 days, about 6 days, about 7 days, about 8 days, about 9 days, about 10 days, about 11 days, about 12 days, about 13 days, about 14 days, about 20 days, about 28 days, about a week, about 2 weeks, about 3 weeks, about 4 weeks, about a month, about every 2 months, about every 3 months, about every 4 months, about every 5 months, about every 6 months, about every 7 months, about every 8 months, about every 9 months, about every 10 months, about every 11 months, about every year, about every 2 years, about every 3 years, about every 4 years, or about every 5 years.

For example, an immune checkpoint inhibitor or inactivated MVA or MVA ΔE3L may be administered daily, weekly, biweekly, or monthly for a particular period of time. An immune checkpoint inhibitor or inactivated MVA or MVA ΔE3L may be dosed daily over a 14 day time period, or twice daily over a seven day time period. An immune checkpoint inhibitor or inactivated MVA or MVA ΔE3L may be administered daily for 7 days.

Alternatively, an immune checkpoint inhibitor or inactivated MVA or MVA ΔE3L may be administered daily, weekly, biweekly, or monthly for a particular period of time followed by a particular period of non-treatment. In some embodiments, the immune checkpoint inhibitor or inactivated MVA or MVA ΔE3L can be administered daily for 14 days followed by seven days of non-treatment, and repeated for two more cycles of daily administration for 14 days followed by seven days of non-treatment. In some embodiments, the immune checkpoint inhibitor or inactivated MVA or MVA ΔE3L can be administered twice daily for seven days followed by 14 days of non-treatment, which may be repeated for one or two more cycles of twice daily administration for seven days followed by 14 days of non-treatment.

In some embodiments, the immune checkpoint inhibitor or inactivated MVA or MVA ΔE3L is administered daily over a period of 14 days. In another embodiment, the immune checkpoint inhibitor or inactivated MVA or MVA ΔE3L is administered daily over a period of 12 days, or 11 days, or 10 days, or nine days, or eight days. In another embodiment, the immune checkpoint inhibitor or inactivated MVA or MVA ΔE3L is administered daily over a period of seven days. In another embodiment, the immune checkpoint inhibitor or inactivated MVA or MVA ΔE3L is administered daily over a period of six days, or five days, or four days, or three days.

In some embodiments, individual doses of the immune checkpoint inhibitor and the inactivated MVA or MVA ΔE3L are administered within a time interval such that the two inhibitors can work together (e.g., within 1 hour, 2 hours, 4 hours, 6 hours, 12 hours, 24 hours, 48 hours, 72 hours, 96 hours, 5 days, 6 days, 1 week, or 2 weeks). In some embodiments, the treatment period during which the therapeutic agents are administered is then followed by a non-treatment period of a particular time duration, during which the therapeutic agents are not administered to the patient. This non-treatment period can then be followed by a series of subsequent treatment and non-treatment periods of the same or different frequencies for the same or different lengths of time. In some embodiments, the treatment and non-treatment periods are alternated. It will be understood that the period of treatment in cycling therapy may continue until the patient has achieved a complete response or a partial response, at which point the treatment may be stopped. Alternatively, the period of treatment in cycling therapy may continue until the patient has achieved a complete response or a partial response, at which point the period of treatment may continue for a particular number of cycles. In some embodiments, the length of the period of treatment may be a particular number of cycles, regardless of patient response. In some other embodiments, the length of the period of treatment may continue until the patient relapses.

In some embodiments, the immune checkpoint inhibitor and the inactivated MVA or MVA ΔE3L are cyclically administered to a patient. Cycling therapy involves the administration of a first agent (e.g., a first prophylactic or therapeutic agent) for a period of time, followed by the administration of a second agent and/or third agent (e.g., a second and/or third prophylactic or therapeutic agent) for a period of time and repeating this sequential administration. Cycling therapy can reduce the development of resistance to one or more of the therapies, avoid or reduce the side effects of one of the therapies, and/or improve the efficacy of the treatment.

In some embodiments, the immune checkpoint inhibitor is administered for a particular length of time prior to administration of the inactivated MVA or MVA ΔE3L. For example, in a 21-day cycle, the immune checkpoint inhibitor may be administered on days 1 to 5, days 1 to 7, days 1 to 10, or days 1 to 14, and the inactivated MVA or MVA ΔE3L may be administered on days 6 to 21, days 8 to 21, days 11 to 21, or days 15 to 21. In other embodiments, the inactivated MVA or MVA ΔE3L is administered for a particular length of time prior to administration of the immune checkpoint inhibitor. For example, in a 21-day cycle, the inactivated MVA or MVA ΔE3L may be administered on days 1 to 5, days 1 to 7, days 1 to 10, or days 1 to 14, and the immune checkpoint inhibitor may be administered on days 6 to 21, days 8 to 21, days 11 to 21, or days 15 to 21.

In one embodiment, the administration is on a 21-day dose schedule in which a once daily dose of immune checkpoint inhibitor is administered beginning on day eight for seven days, followed by seven days of non-treatment, in combination with twice-daily administration of the inactivated MVA or MVA ΔE3L for seven days followed by 14 days of non-treatment (e.g., the immune checkpoint inhibitor is administered on days 8-14 and the inactivated MVA or MVA ΔE3L is administered on days 1-7 of the 21-day schedule). In another embodiment, the administration is on a 21-day dose schedule in which a once daily dose of inactivated MVA or MVA ΔE3L is administered beginning on day eight for seven days, followed by seven days of non-treatment, in combination with twice-daily administration of the immune checkpoint inhibitor for seven days followed by 14 days of non-treatment (e.g., the inactivated MVA or MVA ΔE3L is administered on days 8-14 and the immune checkpoint inhibitor is administered on days 1-7 of the 21-day schedule).

In some embodiments, the immune checkpoint inhibitor and inactivated MVA or MVA ΔE3L each are administered at a dose and schedule typically used for that agent during monotherapy. In other embodiments, when the immune checkpoint inhibitor and inactivated MVA or MVA ΔE3L are administered concomitantly, one or both of the agents can advantageously be administered at a lower dose than typically administered when the agent is used during monotherapy, such that the dose falls below the threshold that an adverse side effect is elicited.

The therapeutically effective amounts or suitable dosages of the immune checkpoint inhibitor and the inactivated MVA or MVA ΔE3L in combination depends upon a number of factors, including the nature of the severity of the condition to be treated, the particular inhibitor, the route of administration and the age, weight, general health, and response of the individual patient. In certain embodiments, the suitable dose level is one that achieves a therapeutic response as measured by tumor regression or other standard measures of disease progression, progression free survival, or overall survival. In other embodiments, the suitable dose level is one that achieves this therapeutic response and also minimizes any side effects associated with the administration of the therapeutic agent.

Suitable daily dosages of immune checkpoint inhibitors can generally range, in single or divided or multiple doses, from about 10% to about 120% of the maximum tolerated dose as a single agent. In certain embodiments, the suitable dosages of immune checkpoint inhibitors are from about 20% to about 100% of the maximum tolerated dose as a single agent. In other embodiments, the suitable dosages of immune checkpoint inhibitors are from about 25% to about 90% of the maximum tolerated dose as a single agent. In some embodiments, the suitable dosages of immune checkpoint inhibitors are from about 30% to about 80% of the maximum tolerated dose as a single agent. In other embodiments, the suitable dosages of immune checkpoint inhibitors are from about 40% to about 75% of the maximum tolerated dose as a single agent. In some embodiments, the suitable dosages of immune checkpoint inhibitors are from about 45% to about 60% of the maximum tolerated dose as a single agent. In other embodiments, suitable dosages of immune checkpoint inhibitors are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

Suitable daily dosages of inactivated MVA or MVA ΔE3L can generally range, in single or divided or multiple doses, from about 10% to about 120% of the maximum tolerated dose as a single agent. In certain embodiments, the suitable dosages of inactivated MVA or MVA ΔE3L are from about 20% to about 100% of the maximum tolerated dose as a single agent. In some other embodiments, the suitable dosages of inactivated MVA or MVA ΔE3L are from about 25% to about 90% of the maximum tolerated dose as a single agent. In some other embodiments, the suitable dosages of inactivated MVA or MVA ΔE3L are from about 30% to about 80% of the maximum tolerated dose as a single agent. In some other embodiments, the suitable dosages of inactivated MVA or MVA ΔE3L are from about 40% to about 75% of the maximum tolerated dose as a single agent. In some other embodiments, the suitable dosages of inactivated MVA or MVA ΔE3L are from about 45% to about 60% of the maximum tolerated dose as a single agent. In other embodiments, suitable dosages of inactivated MVA or MVA ΔE3L are about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, or about 120% of the maximum tolerated dose as a single agent.

### Kits

The present disclosure provides kits for treating PRC2 mutant cancers comprising an immune checkpoint inhibitor disclosed herein, an inactivated MVA or MVA ΔE3L disclosed herein, and instructions for treating PRC2 mutant cancers. When simultaneous administration is contemplated, the kit may comprise an immune checkpoint inhibitor and an inactivated MVA or MVA ΔE3L that has been formulated into a single pharmaceutical composition such as a tablet, or as separate pharmaceutical compositions. When the immune checkpoint inhibitor and the inactivated MVA or MVA ΔE3L are not administered simultaneously, the kit may comprise an immune checkpoint inhibitor and an inactivated MVA or MVA ΔE3L that has been formulated as separate pharmaceutical compositions either in a single package, or in separate packages.

The kits may further comprise pharmaceutically acceptable excipients, diluents, or carriers that are compatible with one or more kit components described herein. Optionally, the above described components of the kits of the present technology are packed in suitable containers and labeled for the treatment of PRC2 mutant cancers. The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way.

### Example 1: Materials and Methods

Human tumor tissue collection. Clinical samples were collected during surgical resection from patients diagnosed as MPNST according to Memorial Sloan Kettering Cancer Center (MSKCC) Institutional Review Board (IRB) protocol. All patients provided informed consent. Frozen and paraffin embedded tissue samples were banked, and tissue microarrays (TMAs) were generated. All MPNST were pathologically reviewed and confirmed by a sarcoma expert (C.R.A.) at MSKCC.

Cell lines. HEK-293T and sNF96.2 cell lines were purchased from ATCC. The human MPNST cell line M3 (NF1^{-/-}, CDKN2A/B^{-/-}) was developed from NF1-associated MPNT patient and gifted by William Gerald. The mouse MPNST cell line SKP605 (Nf1^{-/-}, Cdkn2a/b^{-/-}) was generated from skin-derived precursors according to the published protocol and confirmed with pathologist. All cells were tested to be mycoplasma free by the MycoAlertPlus MycoPlasma Detection Kit (Lonza). All these cells were cultured in DMEM supplemented with L-glutamine (2 mM), penicillin (100 U/ml), and streptomycin (100 μg/ml), and 10% heat-inactivated fetal bovine serum in 5% CO₂ incubator at 37° C.

Immunohistochemistry (IHC). Human tissue processing, embedding, sectioning and staining with hematoxylin and eosin were performed by the MSKCC Department of Pathology. IHC of human TMA tumor samples were performed by the MSKCC Human Oncology and Pathogenesis Program automatic staining facility using a Ventana BenchMark ULTRA Automated Stainer. Mouse fresh tissues were fixed with 4% PFA overnight, washed with PBS 3 time, 5 min for each time, and stored in 70% ethanol. Tissue paraffin embedding, sectioning, and H&E staining were performed by the Histoserv, Inc. IHC was performed by the Molecular Cytology Core Facility at MSKCC for immune marker CD45, or Ventana BenchMark ULTRA Automated Stainer for H3K27me3 (Millipore #07-449, 1:500, protocol #313), Ki67 (Abcam #ab15580, 1:500, protocol #312) and S100B (Abcam #ab52642, 1:2000, protocol #313). Slides were scanned by the Molecular Cytology Core Facility at MSKCC and analyzed using CaseViewer software.

Gene knockout by CRISPR/Cas9. pLCP2B plasmid was generated by removing Cas9-P2A-tRFP from pL-CRISPR.EFS.tRFP (Addgene, #57819) and replacing with Cas9-P2A-Blast in our lab. LentiCRISPR-v2 vector with puromycin was purchased from Addgene #52961. The sgRNA oligos as below were engineered into vectors followed the standard CRISPR/Cas9 knockout protocol. M3 sgCon cells were pooled from 6 single clones, while M3 sgSUZ12 cells were pooled from 9 single clones. Both SKP605 sgCon cells and sgEed cells were 4 single clones pooled. The doubling time for M3 cells is about 48 hours, while the doubling time for SKP605 is about 24 hr. The cells will be used for experiments within 1 month (15-30 passages) after pooling single clones.

pLCP2B plasmid was generated by removing Cas9-P2A-tRFP from pL-CRISPR.EFS.tRFP (Addgene, #57819) and replacing with Cas9-P2A-Blast in our lab. lentiCRISPR-v2 vector with puromycin was purchased from Addgene #52961. The sgRNA oligos as below were annealed, digested using BsmBI (New England BioLab, #R0580L) and cloned into vectors, such as pLCP2B or lentiCRISPR-v2. To make lentivirus, the transfer plasmid was co-transfected into HEK-293T cells with the packaging plasmids pVSVg (Addgene, #8454) and psPAX2 (Addgene, #12260). To delete target genes, cells were transduced with respective sgRNA and selected with 5-10 μg/ml blasticidin S HCl (Thermo Fisher, #A1113903) or 2-5 μg/ml Puromycin Dihydrochloride (Thermo Fisher, #A1113803) until all negative control cells were dead. Then M3 sgCon were M3 sgSUZ12 cells were seeded at super-low density to allow colony formation from single cells in 96-well plates. Colonies were then picked and expanded for knockout validation by immunoblotting based on H3K27me3 level. Similarly, sgNf1 and sgCdkn2a were cloned into the pLCP2B and blasticidin S HCl selected; sgCdkn2b was cloned in LentiCRISPRv2-mCherry vector (Addgene, #99154) in SKP cells. sgEed was cloned into lentiCRISPR-v2, and SKP605 cells were puromycin selected and single clone picked. M3 sgCon cells were pooled from 6 single clones, while M3 sgSUZ12 cells were pooled from 9 single clones. Both SKP605 sgCon cells and sgEed cells were 4 single clones pooled. The doubling time for M3 cells is about 48 hours, while the doubling time for SKP605 is about 24 hr. The cells will be used for experiments within 1 month (15-30 passages) after pooling single clones.

sgRNA Oligos are as following Sequence
(5′ to 3′) and their PAM are NGG:
sgCon (Control):
F,
(SEQ ID NO: 1)
caccgGCTGATCTATCGCGGTCGTC,
R,
(SEQ ID NO: 2)
aaacGACGACCGCGATAGATCAGCc;
Human sgSUZ12_VEFS:
F,
(SEQ ID NO: 3)
caccgTCCATTTCTTGTGGACGGAG,
R,
(SEQ ID NO: 4)
aaacCTCCGTCCACAAGAAATGGAc;
Mouse_sgEed-1_WD40:
F,
(SEQ ID NO: 5)
accgGAAGGTTTGGGTCTCGTGGG,
R,
(SEQ ID NO: 6)
aaacCCCACGAGACCCAAACCTTCc;
Mouse_sgEed-2_WD40:
F,
(SEQ ID NO: 7)
caccgGGAGAAGGTTTGGGTCTCGT,
R,
(SEQ ID NO: 8)
aaacACGAGACCCAAACCTTCTCCc;
Mouse_sgSuz12_VEFS:
F,
(SEQ ID NO: 9)
tcccGAATTTTCTGATGTGAATGA,
R,
(SEQ ID NO: 10)
aaacTCATTCACATCAGAAAATTC;
Mouse_Nf1:
F,
(SEQ ID NO: 11)
caccgGGGAGAACTCCCTATAGCTA,
R,
(SEQ ID NO: 12)
aaacTAGCTATAGGGAGTTCTCCCc;
Mouse_sgCdkn2a:
F,
(SEQ ID NO: 13)
caccgCGGTGCAGATTCGAACTGCG,
R,
(SEQ ID NO: 14)
aaacCGCAGTTCGAATCTGCACCGc;
Mouse_sgCdkn2b:
F,
(SEQ ID NO: 15)
caccgTTGGGCGGCAGCAGTGACGC,
R,
(SEQ ID NO: 16)
aaacGCGTCACTGCTGCCGCCCAAc

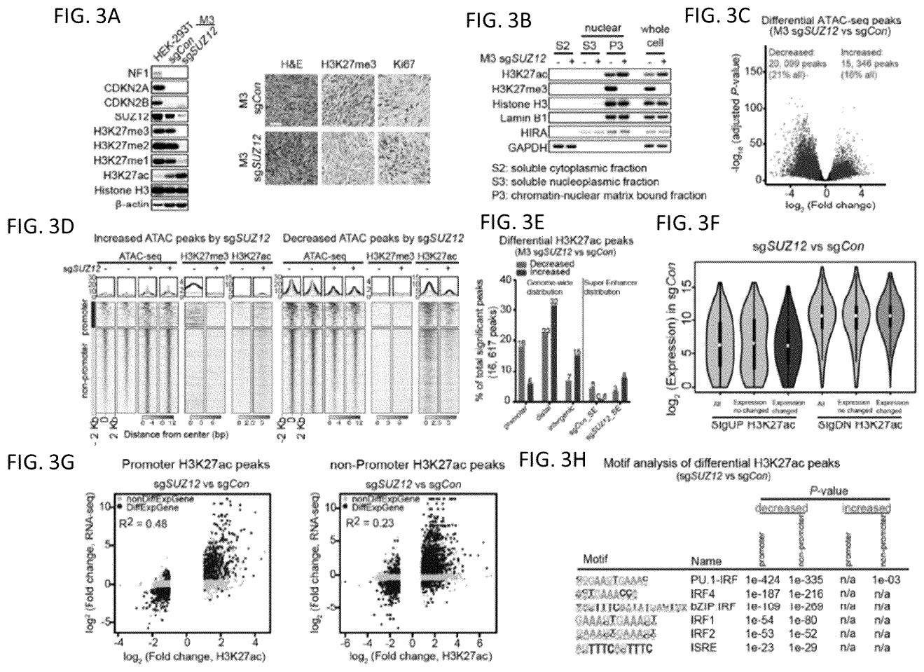

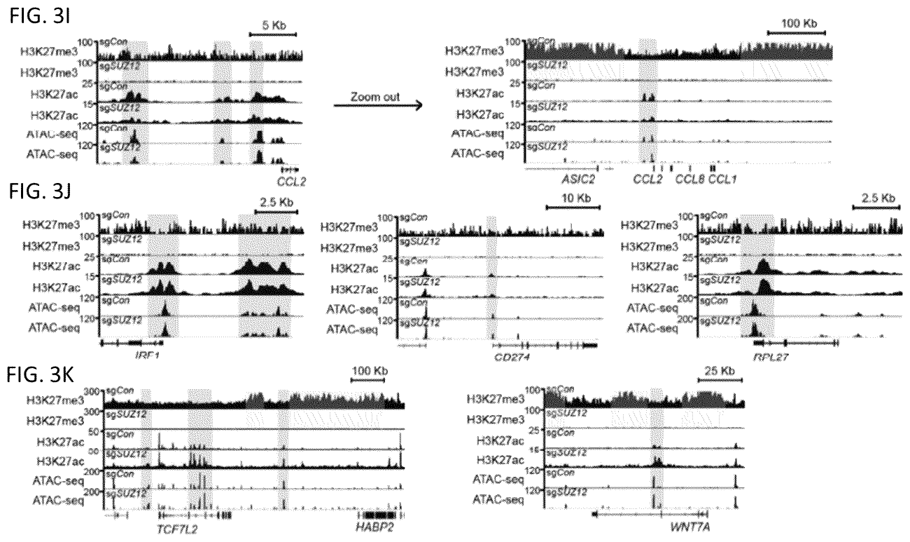

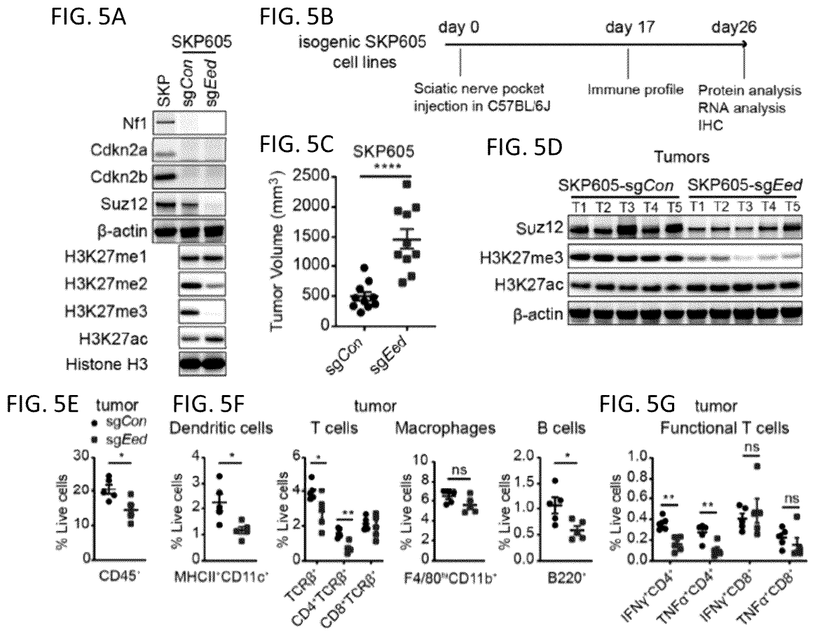

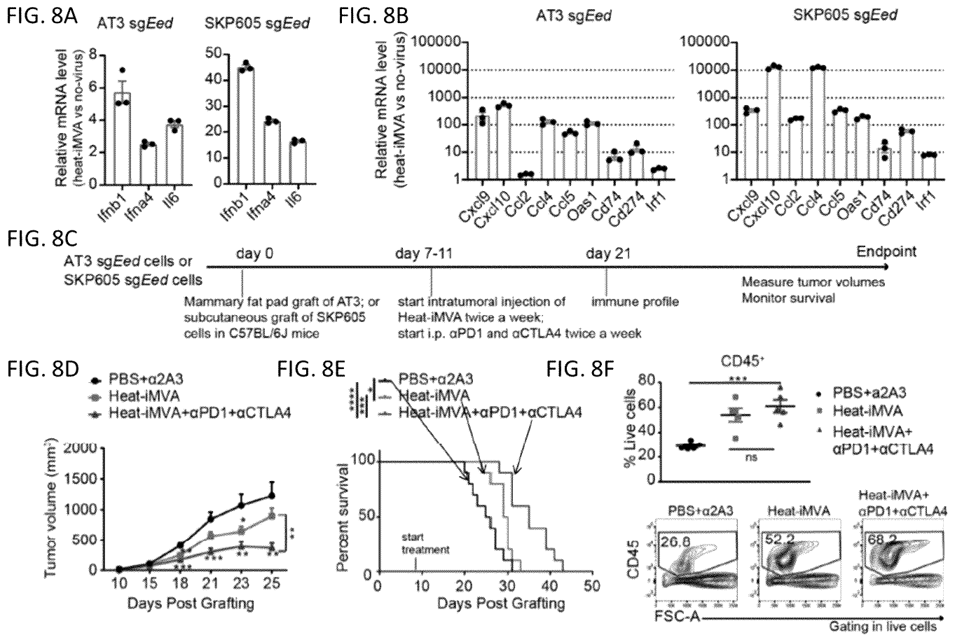

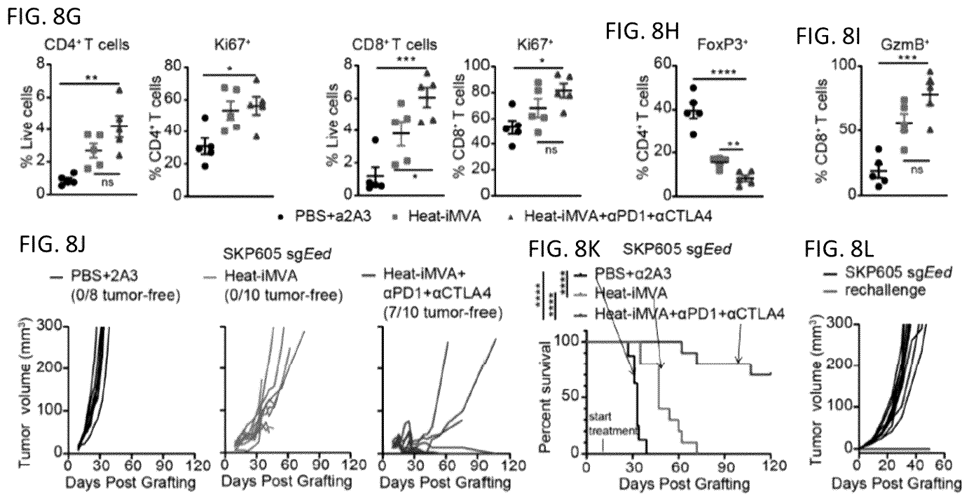

Transplant mouse model. Wild-type 6-8-week-old female C57BL/6J mice were purchased from the Jackson Laboratory (Stock No: 000664), while 6-8-week-old female NSG mice were purchased from MSKCC core facility. All the procedures related to mouse handling, care and the treatment were performed according the guidelines from the MSKCC approved by the Institutional Animal Care and Use Committee (IACUC). Animal protocols were approved by the MSKCC. Isogenic M3 cells were orthotopically transplanted into sciatic nerve pockets of NSG mice: 3 million cells in 100 μl precooling 1:1 PBS:Matrigel (Corning, #356237) in FIG. 3A. Isogenic SKP605 cells were orthotopically transplanted into sciatic nerve pockets of C57BL/6J mice: 5 million cells in 100 μl precooling 1:1 PBS:Matrigel in FIG. 5, while subcutaneously grafted on flanks of C57BL/6J mice: 1 million cells in 100 μl PBS in FIG. 8 for easier MVA treatment and tumor monitoring. Isogenic AT3 cells were orthotopically transplanted into mammary fat pads of C57BL/6J mice: 100 to 150 thousand cells in 100 μl precooling PBS. Staples were removed at least after 1 week of grafting. Tumors were measured twice a week by Vernier caliper. Tumor volume (TV)=4/3*pi*length/2*width/2*height/2. For survival study in FIG. 8, mice were euthanized when the tumor volume reached 1500 mm³ for AT3 mammary gland tumors and 300 mm³ for SKP605 subcutaneous tumors, or the mice exhibit signs of illness.

Monoclonal antibody therapy. Monoclonal antibodies anti-PD1, anti-CTLA4 and anti-2A3 were purchased from Bio X cell: InVivoMAb anti-mouse PD-1 (CD279) (#BE0146), InVivoMAb anti-mouse CTLA-4 (CD152) (#BE0164), and InVivoMAb rat IgG2a isotype control, anti-trinitrophenol (Clone: 2A3) (#BE0089). Antibodies were i.p. once every three days at a dose of 250 μg anti-PD1+200 μg anti-CTLA4 or 100 μg anti-2A3 per 100 μl PBS per mouse per treatment. The dose of ICB decreased during combination with immunogenic MVA in FIGS. 8J-8K: 125 μg anti-PD1+100 μg anti-CTLA4.

Monoclonal antibodies for depletion assay were purchased from Bio X cell: InVivoMAb anti-mouse CD4 (Clone: GK1.5) (#BE0003-1), InVivoMAb anti-mouse CD8a (Clone: 2.43) (#BE0061), InVivoMAb anti-mouse NK1.1 (Clone: PK136) (#BE0036), and InVivoMAb anti-mouse IFNγ (Clone: XMG1.2) (#BE0055). Antibodies were i.p. once every three days at a dose of 200 μg in 100 μl PBS per mouse per treatment starting 2 days before ICBs.

Viruses and intratumoral injection with viruses. The MVA virus was kindly provided by G. Sutter (University of Munich) to the Deng laboratory. MVA was propagated in BHK-21 cells (baby hamster kidney cell, American Type Culture Collection (ATCC) CCL-10) and purified through a 36% sucrose cushion. Heat-iMVA was generated by incubating purified MVA virus at 55° C. for 1 hour (Dai et al., Science Immunology 2017). At 7 to 11 days after implantation, tumors were measured and intratumorally injected with heat-iMVA (an equivalent of 4×10⁸ plaque-forming units) in 200 μl or PBS twice weekly when mice were under anesthesia. Mice were monitored daily.

T-cell stimulation. Cell Stimulation Cocktail (plus protein transport inhibitors) (500×) (Thermo Fisher, #00-4975-93) was diluted in T cell medium (RPMI 1640+10% FBS+L-glutamine (2 mM)+penicillin (100 U/ml+streptomycin (100 μg/ml)+55 μM 2-Mercaptoethanol (Thermo Fisher, #21985023)). 2 million cells were suspended in 200 μl T-cell medium with stimulation cocktail in plates, then incubated for 4 hours at 37° incubator. After incubation, cells were harvested, stained with surface markers and then intracellular markers for FACS analysis.

OVA model antigen system. pMSCV-EGFP-PGK-Luc2-2A-USA plasmid includes OT-I binding antigen and OT-II binding antigen for MHC of C57BL/6J mouse background. To make retrovirus, HEK293T cells were transfected with pMSCV-EGFP-PGK-Luc2-2A-USA, packaging plasmids pVSVg (Addgene, #8454) and pEco (Takara, #PT3749-5). Cells transduced with the retrovirus were sorted for EGFP positive by flow cytometry, and these cells stably overexpressed OVA model antigens: OT-I binding antigen and OT-II binding antigen. APC anti-mouse H-2Kb bound to SIINFEKL antibody (BioLegend, #141605) was used to detect OVA model antigen on cancer cell surface. To detect OVA-specific T cell priming, OVA+ and OVA-AT3 cells were orthotopically grafted in mammary fat pads of C57BL/6 mice. After 18 days post grafting, cells isolated from tumor draining lymph nodes (TdLNs) were incubated with 2 μg/ml OVA 257-264 in T cell medium for 24 h at 37° incubator. First, cells were stained with Fc blocking for 15 mins; Second, Fc blocking plus iTAg H-2Kb OVA Tetramer-SIINFEKL-APC (MBL, #TB-5001-2) in FACS buffer for 1 h at room temperature from light. Third, live/dead dye (TONBO, #13-0863-T100) and anti-mouse CD8a-FITC (TONBO, #35-1886-U100) were added into the staining system for 30 mins at 4°. Finally, cells were washed by FACS buffer for 3 times and ready for FACS analysis.

Protein extraction and western blotting. Sample preparation and western blot was performed as previously described and described herein. Primary antibodies for immunoblotting are as below: anti-NF1 (1:2000, Bethyl, #A300-140A), anti-CDKN2A (1:1000, DeltaBioLabs, #DB018), anti-CDKN2B (1:500, Abcam, #ab53034), anti-SUZ12 (1:1000, CST, #3737), anti-H3K27me3 (1:2000, CST, #9733), anti-H3K27me2 (1:5000, CST, #9728), anti-H3K27me1 (1:1000, Takara, #MABI0321-100I), anti-H3K27ac (1:4000, Abcam, #ab4729), anti-Histone H3 (1:2000, CST, #12648), anti-β-actin (1:5000, Proteintech, #66009-1-Ig), anti-Lamin B1 (1:2000, Proteintech, #12987-1-AP), anti-HIRA (1:1000, Active motif, #39557) and anti-GAPDH (1:3000, CST, #5174S).

After washed by precooling PBS, cells were lysed using preheated 2% SDS followed by boiling for 30 min at 95°. Protein concentrations were determined using BCA protein assay kit (Thermo Fisher, #23225). Protein samples were prepared by adding LDS loading buffer (4×) (Thermo Fisher, #NP0008) and 1 M DTT followed by boiling for 30 min at 95°. Proteins were separated by 4-12% Bis-Tris Gel (Thermo Fisher, #NP0336BOX) with IVIES SDS running buffer (20×) (Thermo Fisher, #NP0002), and transferred to the nitrocellulose membrane (BioRad, #1620115). Membranes were blocked with blocking buffer (Thermo Fisher, #UH289384) for 1 hr at room temperature and incubated with the indicated primary antibodies at 4° overnight. After washing with 1× Tris-buffered saline Tween-20 (TBST) (25 mM Tris, 150 mM NaCl, 2 mM KCl, pH 7.4, supplemented with 0.2% Tween-20) three times for 30 min, membranes were incubated with peroxidase-conjugated secondary antibodies at room temperature for 1 hr. The membranes were washed again with TBST three times and visualized with chemiluminescence using HRP substrate (Millipore, #WBKLS0500) or Pico (Thermo Fisher, #34578).

RNA isolation and qRT-PCR. Total RNA was isolated from cell lines using total RNA kit I (Omega, #R6834-02) and homogenizer mini columns (Omega, #HCR003), or tissues using Trizol (Thermo Fisher #15596026). cDNA was prepared using High-Capacity cDNA Reverse Transcription Kit (Thermo Fisher, #4368814). qRT-PCR was performed following the instruction for SYBR Green Master Mix (Thermo Fisher, #A25777) with V7 Real-Time PCR system (Applied Biosystems). Expressed values relative to control were calculated using the ΔΔCT method. Housekeeping gene RPL27 was used as reference gene for normalization.

The sequences of primers used for qPCR analysis
were listed as follows (5′-3′).
Human_RPL27:
F,
(SEQ ID NO: 17)
CATGGGCAAGAAGAAGATCG,
R
(SEQ ID NO: 18)
TCCAAGGGGATATCCACAGA;
Human SUZ12:
F,
(SEQ ID NO: 19)
TTGCAGCTTACGTTTACTGGTT,
R,
(SEQ ID NO: 20)
GGAACTTGCCTTATTGGACAACT;
Human_EED:
F,
(SEQ ID NO: 21)
CTGTAGGAAGCAACAGAGTTACC,
R,
(SEQ ID NO: 22)
CATAGGTCCATGCACAAGTGT;
Human_CD274:
F,
(SEQ ID NO: 23)
TGGCATTTGCTGAACGCATTT,
R,
(SEQ ID NO: 24)
TGCAGCCAGGTCTAATTGTTTT;
Human_IRF1:
F,
(SEQ ID NO: 25)
CTGTGCGAGTGTACCGGATG,
R,
(SEQ ID NO: 26)
ATCCCCACATGACTTCCTCTT;
Human_CCL2:
F,
(SEQ ID NO: 27)
CAGCCAGATGCAATCAATGCC,
R,
(SEQ ID NO: 28)
TGGAATCCTGAACCCACTTCT;
Human_CD74:
F,
(SEQ ID NO: 29)
GATGACCAGCGCGACCTTATC,
R,
(SEQ ID NO: 30)
GTGACTGTCAGTTTGTCCAGC;
Human_CIITA:
F,
(SEQ ID NO: 31)
CCTGGAGCTTCTTAACAGCGA,
R,
(SEQ ID NO: 32)
TGTGTCGGGTTCTGAGTAGAG;
Human_HLA-DRA:
F,
(SEQ ID NO: 33)
AGTCCCTGTGCTAGGATTTTTCA,
R,
(SEQ ID NO: 34)
ACATAAACTCGCCTGATTGGTC;
Human_HLA-DRB:
F,
(SEQ ID NO: 35)
CGGGGTTGGTGAGAGCTTC,
R,
(SEQ ID NO: 36)
AACCACCTGACTTCAATGCTG;
Human_LAP3:
F,
(SEQ ID NO: 37)
GTCTGGCCGTGAGACGTTT,
R,
(SEQ ID NO: 38)
ACCATAAAAGGTTCGAGTCTTCC;
Mouse_Rpl27:
F,
(SEQ ID NO: 39)
AAAGCCGTCATCGTGAAGAAC,
R,
(SEQ ID NO: 40)
GATAGCGGTCAATTCCAGCCA;
Mouse_Eed:
F,
(SEQ ID NO: 41)
AGCCACCCTCTATTAGCAGTT,
R,
(SEQ ID NO: 42)
GCCACAAGAGTGTCTGTTTGGA;
Mouse_Ccl2:
F,
(SEQ ID NO: 43)
TAAAAACCTGGATCGGAACCAAA,
R,
(SEQ ID NO: 44)
GCATTAGCTTCAGATTTACGGGT;
Mouse_Cxcl10:
F,
(SEQ ID NO: 45)
CCAAGTGCTGCCGTCATTTTC,
R,
(SEQ ID NO: 46)
TCCCTATGGCCCTCATTCTCA;
Mouse_Il2:
F,
(SEQ ID NO: 47)
ACCTGAAACTCCCCAGGAT,
R,
(SEQ ID NO: 48)
TCATCGAATTGGCACTCAAA;
Mouse_Oas1:
F,
(SEQ ID NO: 49)
GGGCCTCTAAAGGGGTCAAG,
R,
(SEQ ID NO: 50)
TCAAACTTCACTCCACAACGTC;
Mouse_Cd274:
F,
(SEQ ID NO: 51)
AGTATGGCAGCAACGTCACG,
R,
(SEQ ID NO: 52)
TCCTTTTCCCAGTACACCACTA;
Mouse_Irf1:
F,
(SEQ ID NO: 53)
GGCCGATACAAAGCAGGAGAA,
R,
(SEQ ID NO: 54)
GGAGTTCATGGCACAACGGA;
Mouse_Actb:
F,
(SEQ ID NO: 55)
GTGACGTTGACATCCGTAAAGA,
R,
(SEQ ID NO: 56)
GCCGGACTCATCGTACTCC;
Mouse_Ifnb1:
F,
(SEQ ID NO: 57)
TGGAGATGACGGAGAAGATG;
R,
(SEQ ID NO: 58)
TTGGATGGCAAAGGCAGT;
Mouse_Ifna4:
F,
(SEQ ID NO: 59)
CCTGTGTGATGCAGGAACC,
R,
(SEQ ID NO: 60)
TCACCTCCCAGGCACAGA;
Mouse Il6:
F,
(SEQ ID NO: 61)
AGGCATAACGCACTAGGTTT,
R,
(SEQ ID NO: 62)
AGCTGGAGTCACAGAAGGAG.

RNA-seq and analysis. Total RNA was isolated from fresh tissues or duplicate wells of indicated conditions using Trizol (Thermo Fisher #15596026). RNA-seq library construction and sequencing were performed at the integrated genomics operation (IGO) core facility at MSKCC using poly-A capture. The libraries were sequenced on an Illumina HiSeq-2500 platform with 50-bp paired-end (human MPNST tumors) or single-end (AT3 tumors) reads to obtain a minimum yield of 40 million reads per sample. The sequence data were processed and mapped to the human reference genome (hg19) or mouse reference genome (mm9) using STAR v2.3 [68]. Gene expression was quantified to transcripts-per-million (TPM) using the STAR and Log 2 transformed. GSEA was performed using JAVA GSEA 2.0 program [70].

Assay for transposase-accessible chromatin using sequencing (ATAC-seq) and analysis. Cells were washed with PBS, trypsinized from 6-well plates and harvested as a single-cell suspension in precool PBS. 100 thousand cells were sent to Center for Epigenetics Research at MSKCC for ATAC-seq performed as previously described [72]. For each sample, cell nuclei were prepared from 50,000 cells, and incubated with 2.5 μl of transposase (Illumina) in a 50-μl reaction for 30 min at 37° C. After purification of transposase-fragmented DNA, the library was amplified by PCR and subjected to paired-end 50 base-pair high-throughput sequencing on an Illumina HiSeq2500 platform. For data analysis, ATAC-seq reads were quality and adapter trimmed using Trim Galore before aligning to human genome assembly hg19 with Bowtie2 using the default parameters. Motif signatures were obtained using HOMER v4.5 (http://homer.ucsd.edu).

For data analysis, ATAC-seq reads were quality and adapter trimmed using Trim Galore before aligning to human genome assembly hg19 with Bowtie2 using the default parameters. Aligned reads with the same start position and orientation were collapsed to a single read before subsequent analysis. Density profiles were created using the BEDTools suite, with subsequent normalization to a sequencing depth of ten million reads for each library. To ascertain regions of open chromatin, MACS2 (https://github.com/taoliu/MACS) was used with a p-value setting of 0.001 against a cell line-matched input sample. A global peak atlas was created by first removing blacklisted regions then merging all peaks within 500 bp and counting reads with version 1.6.1 of featureCounts (http://subread.sourceforge.net). Differential enrichment was scored using DESeq2 for all pairwise group contrasts. Differential peaks were then merged into a union set, and k-means clustering was performed from k=4:10, stopping when redundant clusters emerged. Peak-gene associations were created by assigning all intragenic peaks to that gene, and otherwise using linear genomic distance to transcription start site. GSEA (http://software.broadinstitute.org/gsea) was performed with the pre-ranked option and default parameters, where each gene was assigned the single peak with the largest (in magnitude) log 2 fold change associated with it. Motif signatures were obtained using HOMER v4.5 (http://homer.ucsd.edu).

Chromatin Immunoprecipitation sequencing (ChIP-seq), CUT&RUN and analysis. Chromatin isolation from indicated cells and immunoprecipitation were performed as previously described [73]. The libraries were sequenced on an Illumina HiSeq2500 platform with 50-bp paired-end reads. Reads were trimmed by the software Trim Galore and then aligned to the human genome (hg19) using the Bowtie2 alignment software (v2.3.5) [74]. Duplicated reads were eliminated for subsequent analysis. Peak calling for H3K27ac was performed using software MACS2 (v2.1.1) in paired mode and comparing ChIP samples to input, using the false discovery rate of q<10⁻³. H3K27me3 peaks were called by a sliding window approach to find regions enriched with H3K27me3 compared to input reads. Spike-in was used in H3K27me3 ChIP-seq for normalization because of the global loss of H3K27me3 after PRC2 loss. We discarded peaks mapped to blacklisted genomic regions identified by the ENCODE [76] [77]. Primary antibodies for ChIP are as below: anti-H3K27me3 (CST, #9733) and anti-H3K27ac (Abcam, #ab4729).

The H3K27ac peaks were separated into promoters, distal and intergenic peaks as described previously [78]. They were also used for super enhancer (SE) analysis using the ROSE R package (option: −t 2500)[79] [80]. The peaks from controls and sgSUZ12 knockout samples were merged to generate a non-overlapping list of union peaks. ChIP-seq reads located to the merged peaks were calculated and used by the software DESeq2 to identify peaks with differentially modifications at the adjusted p-value <0.05 and fold change >2. The significantly increased or decreased peaks in the sgSUZ12 samples at promoters and non-promoter regions were subject to independent transcription factor binding motif analysis with the HOMER software (v4.7, default parameter)[81], using all peaks as background. The fold changes in these peaks were also compared to gene expression changes in the RNA-seq analysis for each of the promoter and distal peaks assigned to genes (note that one gene could have multiple peaks). CUT&RUN assay was performed using the commercial kit and protocol (CST, #86652). Primary antibodies (1 μg per assay) for CUT&RUN are as below: anti-H3K27me3 (CST, #9733), anti-H3K27ac (Abcam #ab4729), anti-H3K37me2 (Thermo Fisher Scientific, #MA5-14867), and anti-H3K36me3 (Active motif, #61021).

Data availability. RNA-seq data of human MPNST tumor tissues were deposited in dbGaP with study accession: phs000792.v1.p1. Other sequencing data were deposited in the Gene Expression Omnibus: RNA-seq data of AT3 tumors in GSE179703; ATAC-seq data of M3 cells in GSE179699; ChIP-seq data of M3 cells in GSM5420909, GSM5420912, GSM5420919 and GSM5420922; CUT&RUN data of M3 cells in GSE202555.

Statistics. All statistical analyses were performed using GraphPad Prism 7. Unless otherwise noted in the figure legend, all data were shown as mean±SEM combined with a two-tailed unpaired t-test for statistical comparisons between two groups, one-way ANOVA for more than two groups, and Log-rank (Mantel-Cox) test for survival study. Significance was defined as below: *P<0.05, **P<0.01, ***P<0.001, ****P<0.0001. All experiments shown were repeated in at least two independent experiments.

Study approval. Clinical samples were collected during surgical resection from patients diagnosed as MPNST according to Memorial Sloan Kettering Cancer Center (MSKCC) Institutional Review Board (IRB) protocol. All the procedures related to mouse handling, care and the treatment were performed according the guidelines from the MSKCC approved by the Institutional Animal Care and Use Committee (IACUC). Animal protocols were approved by the MSKCC.

Tumor infiltrate analysis by flow cytometry. Fresh tumor tissues were minced with razor blades on slides one by one, then digested with 10 ml digestion buffer separately (MPNST tumors: 200 U/ml Collagenase I+300 U/ml Collagenase IV+4 μg/ml DNase I+RPMI-1640, breast cancer tumors: 300 U/ml Collagenase III+4 μg/ml DNase I+HBSS). All these enzymes were purchased from Worthington: Collagenase I #LS004196, Collagenase III #LS004182, Collagenase IV #LS004188, and DNase I #LS002006. HBSS buffer was purchased from Thermo Fisher Scientific #14025076. Samples were incubated for 1 hour in 37° C. shaker. To get single cells, tumors were mashed through 70 micron filters. To enrich live cells, Ficoll (Thermo Fisher Scientific, #17144002) centrifugation was used with subsequent washing of the obtained cells. Spleens were minced directly and lysed in ACK lysis buffer (Thermo Fisher Scientific, #A1049201) to remove red cells. For flow cytometric analysis, about 1.5 million washed cells were resuspended in 70 μl FACS buffer (2% FBS+1 mM EDTA (0.5M, 1:500)+10 mM HEPES (1M, 1:100) in PBS) with Fc block for 15 min on ice first, then Fc block plus indicated antibodies for 30 min on ice from light. Foxp3/Transcription Factor Staining Buffer Kit (Tonbo, #TNB-0607-KIT) was used to stain intracellular markers. All FACS were performed on BD LSR Fortessa, and data were analyzed using FlowJo software. Antibodies for FACS are as below: Purified Anti-Mouse CD16/CD32 (1:500, 2.4G2) (TONBO, #70-0161-U100), Ghost Dye™ Violet 450 (1:1000, TONBO, #13-0863-T100), PE-Texas Red_CD45 (1:200, Thermo Fisher Scientific, #MCD4517), APC/Cy7_TCR β (1:200, Biolegend, #109220), PerCP-Cyanine5.5_CD8a (1:200, TONBO, #65-1886-U100), BV510_CD4 (1:200, BD, #563106), PE_B220 (1:400, TONBO, #50-0452-U100), FITC_F4/80 (1:200, TONBO, #35-4801-U100), APC-Cyanine7_CD11b (1:200, TONBO, #25-0112-U100), PE-Cyanine7_CD11c (1:200, TONBO, #60-0114-U025), APC_MHC Class II (I-A/I-E) (1:600, TONBO, #20-5321-U100), FITC_IFN gamma (1:100, TONBO, #35-7311-U100), PE-Cyanine7_TNF alpha (1:100, Thermo Fisher Scientific, #25-7423-82).

Cell colony formation assay. AT3 cells were seeded into 6-well plates at a concentration of 200 cells per well. After 9 days. colonies were fixed with fixation solution (10% methanol+10% acetic acid) at room temperature for 15 min and then stained with a solution of 1% crystal violet in methanol for 15 min.

### Example 2: Tumor-Intrinsic PRC2 Loss is Associated with Immune-Desert Tumor Microenvironment in MPNST and Other Cancer

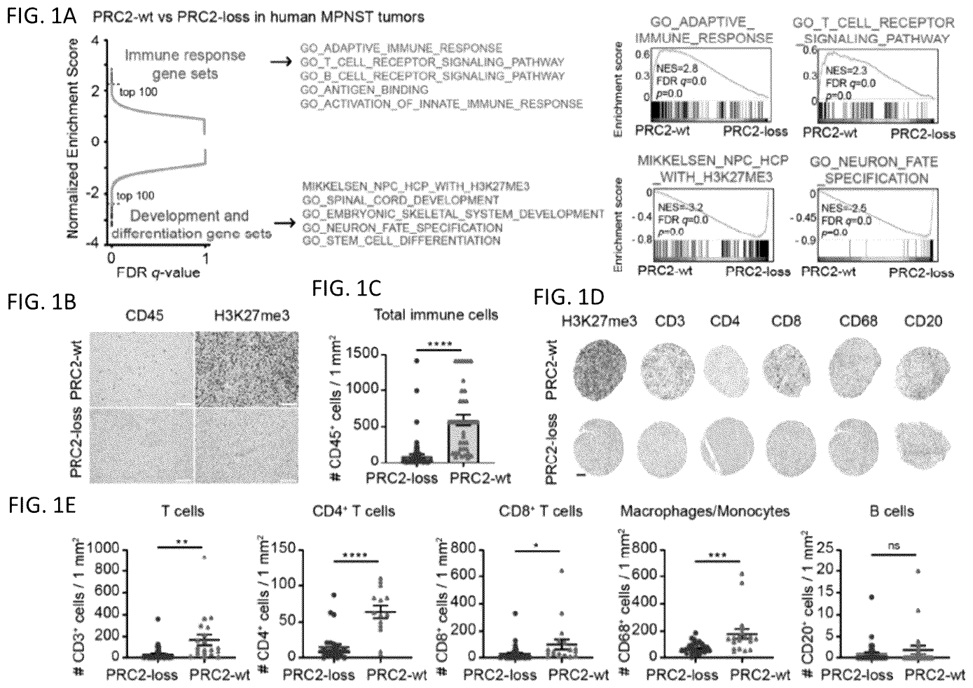

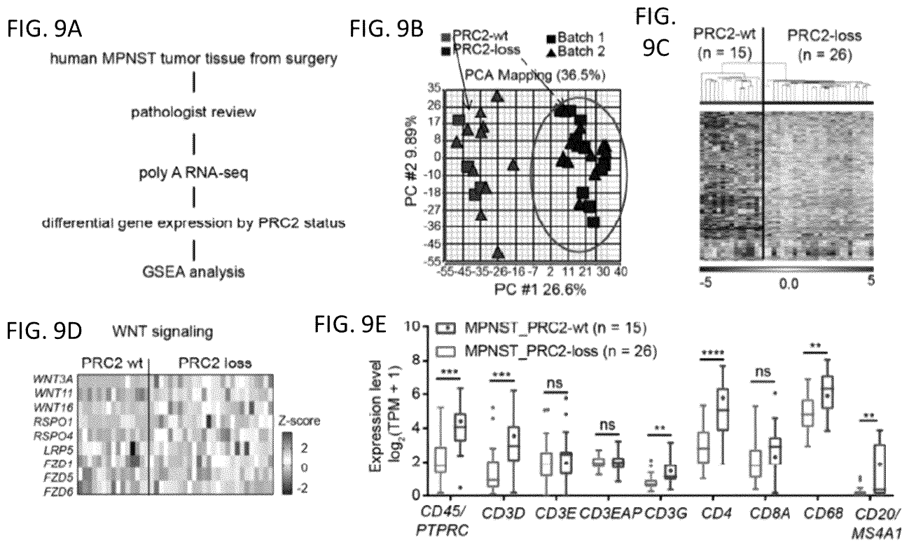

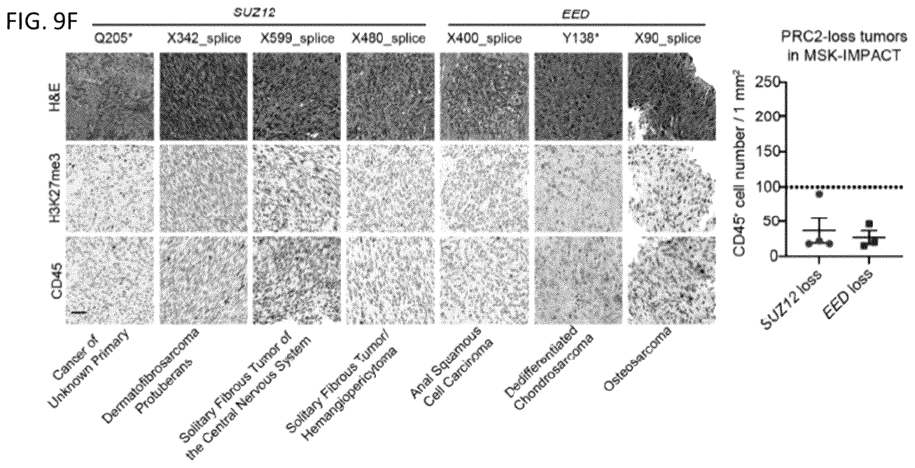

To characterize the role of PRC2 inactivation in cancer pathogenesis, we analyzed the transcriptomes of 41 histologically confirmed high-grade human MPNST tumor samples, consisting of both PRC2-wild-type (wt) and PRC2-loss samples. PRC2 loss in MPNSTs was confirmed by loss of H3K27me3 immunostaining and/or genetic inactivation of EED or SUZ12 by MSK-IMPACT (FIG. 9A and data not shown). Principal component analysis (PCA), which detects sources of variation, showed that the MPNST samples were readily separated by PRC2 status in the first principal component (PC1) (FIG. 9B). We generated a gene set composed of genes that were differentially expressed between PRC2-loss and PRC2-wt samples. Hierarchical clustering based on these genes robustly separated the PRC2-loss and PRC2-wt MPNSTs, with the majority of the most differentially expressed genes upregulated in PRC2-loss compared to PRC2-wt MPNSTs, consistent with the role of PRC2 in transcriptional repression (FIG. 9C). Consistently, gene set enrichment analysis (GSEA) showed that the most enriched pathways and gene sets in PRC2-loss MPNSTs included the PRC2 modules and H3K27me3 target genes, organ development and morphogenesis, neuron cell fate specification and WNT signaling gene sets [3, 31-33] (FIG. 1A, FIG. 9D and data not shown). A distinct smaller subset of genes was consistently downregulated in PRC2-loss compared to PRC2-wt MPNSTs. Remarkably, nearly all these genes were associated with immune function, including both innate and adaptive immune response pathways, T- and B-cell receptor signaling pathways, antigen binding and presentation (FIG. 1A, FIG. 9C).

We next performed immunostaining of several established markers of distinct immune subclasses by immunohistochemistry (IHC) in human MPNST tumor tissue, including CD45 (pan-leukocyte), CD3 (T cells, CD4⁺ and CD8⁺ subsets), CD68 (macrophages/monocytes) and CD20 (B cells). Quantification of each immune subset revealed that compared to PRC2-wt, the PRC2-loss MPNSTs were associated with significant reductions in CD45⁺ leukocytes (FIGS. 1B-1C), CD3⁺ T cells, CD4⁺ and CD8⁺ T cells, and CD68⁺ macrophages/monocytes (FIGS. 1D-1E). Although not significantly different, CD20⁺ B cells were only marginally present in both PRC2-wt and PRC2-loss MPNSTs (FIG. 1E). These data corroborate the transcriptome results of diminished tumor immune infiltrates (FIG. 9E). To further corroborate that PRC2 inactivation in various cancer context are associated with a cold TME, we identified available archival tissues with confirmed PRC2 inactivation through EED or SUZ12 loss-of-function mutations by MSK-IMPACT and loss of H3K27me3 stains by IHC (FIG. 9F and data not shown). IHC of CD45⁺ leukocytes demonstrated that these PRC2-tumors had low levels of tumor immune infiltrates, comparable to those of PRC2-loss MPNSTs (FIG. 9F and FIG. 1C). These results suggest that tumor cell-intrinsic PRC2 inactivation may exclude immune infiltrates and drive an immune-desert TME in cancer.

### Example 3: Antigen Presentation and IFNγ Signaling are Suppressed in PRC2-Loss MPNST

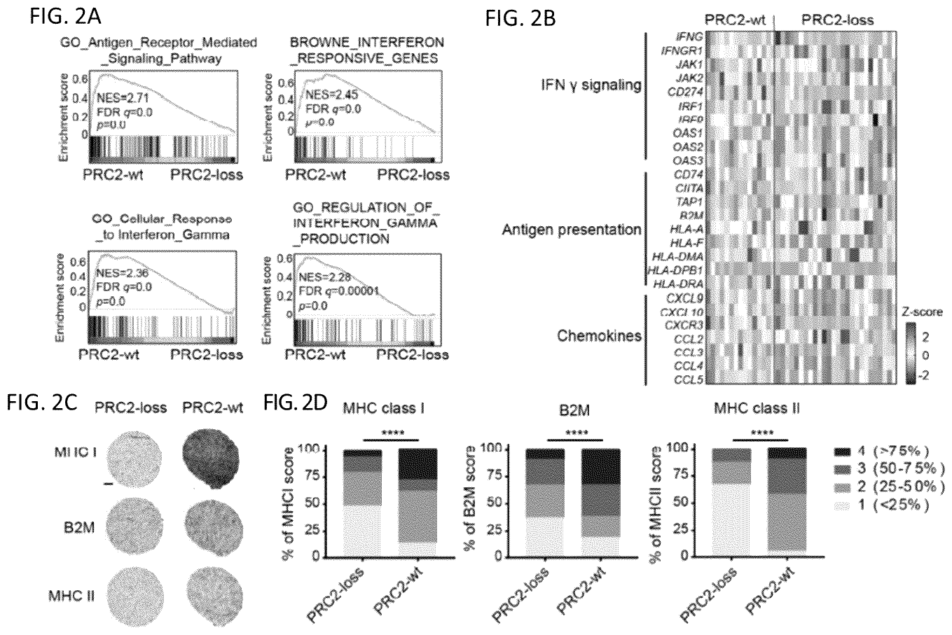

To understand how tumor-intrinsic PRC2 loss leads to an immune-desert TME, we further characterized the transcriptomes of PRC2-loss vs. PRC2-wt MPNST tumors and focused on the initiating steps of anti-tumor immune response [34]. Among the genes downregulated by PRC2 loss, antigen processing and presentation was one of the most negatively enriched gene sets by GSEA (FIG. 2A). Interferon gamma (IFNγ) is an established key regulator of antigen presentation and chemokines for immune cell recruitment [35, 36]. Consistently, IFNγ signaling was significantly impaired in PRC2-loss tumors, including cellular response to IFNγ, interferon responsive genes and regulation of IFNγ production (FIG. 2A). The expression of the tumor cell-autonomous genes, IFNGR1 and downstream signaling axis and effectors, including JAK1, JAK2, IRF1, were all significantly lower in PRC2-loss compared to PRC2-wt tumors (P<0.05) (FIG. 2B). In addition, IFNγ, usually produced by immune cells, was very low in most PRC2-loss tumors (FIG. 2B), consistent with PRC2-loss associated immune-desert phenotype. Overall, the expression of the IFNG-IFNGR1-JAK signaling axis genes was significantly reduced in PRC2-loss compared to PRC2-wt tumors. Consequently, genes relevant to antigen processing and presentation were also decreased accompanying PRC2 loss, including MHC class I (e.g., HLA-A and B2M), MHC class II (e.g., CD74 and HLA-DMA) and antigen processing genes (e.g., TAP1) (FIG. 2B), suggesting decreased tumor immunogenicity in PRC2-loss compared to PRC2-wt tumors. We further validated the decreased protein expression of MHC class I, B2M and MHC class II by IHC in PRC2-loss tumors using MPNST TMA (FIGS. 2C-2D). Furthermore, the key chemokines responsible for immune cell recruitment were significantly lower in PRC2-loss compared to PRC2-wt tumors, including CXCL9/10 (P<0.01) and CCL2/3/4/5 (P<0.05) (FIG. 2B). These data further posit that the tumor cell-intrinsic PRC2 loss-associated immune-desert phenotype is driven by impaired antigen presentation and diminished IFNγ signaling in tumors.

### Example 4: PRC2 Loss Reprograms the Chromatin Landscape and Suppresses a Subset of IFN-Responsive Genes

To examine the role of PRC2 loss in MPNST while minimizing cell line-specific confounding factors, we generated and validated PRC2-isogenic human MPNST cells using CRISPR/Cas9-mediated knockout of the PRC2 core component, SUZ12, in a PRC2-wt, NF1^{-/-}; CDKN2A^{-/-} M3 cell line derived from a human NF1-associated MPNST (FIG. 3A). SUZ12 loss led to a global reduction of the H3K27me3, H3K27me2 and H3K27me1 marks, and a reciprocal global increase of the H3K27ac mark in PRC2-isogenic human MPNST cells (sgCon vs. sgSUZ12) (FIG. 3A). The PRC2-isogenic M3 cells, when orthotopically transplanted into the sciatic nerve pockets of immunodeficient NSG mice, gave rise to high-grade MPNSTs with histological (e.g., monotonous spindle cell morphology, herringbone pattern and fascicular growth) and immunostaining (e.g., H3K27me3 and Ki67 staining) features resembling those of high-grade human MPNST (FIG. 3A). Nucleoplasmic and chromatin fractionation demonstrated that the global decrease of H3K27me3 and increase of H3K27ac occurred on chromatin in SUZ12-loss cells (FIG. 3B). These characterizations combined with the loss of H3K27me3 immunostaining in PRC2-loss M3 cell line-derived MPNST tumors validated the model system for mechanistic studies.

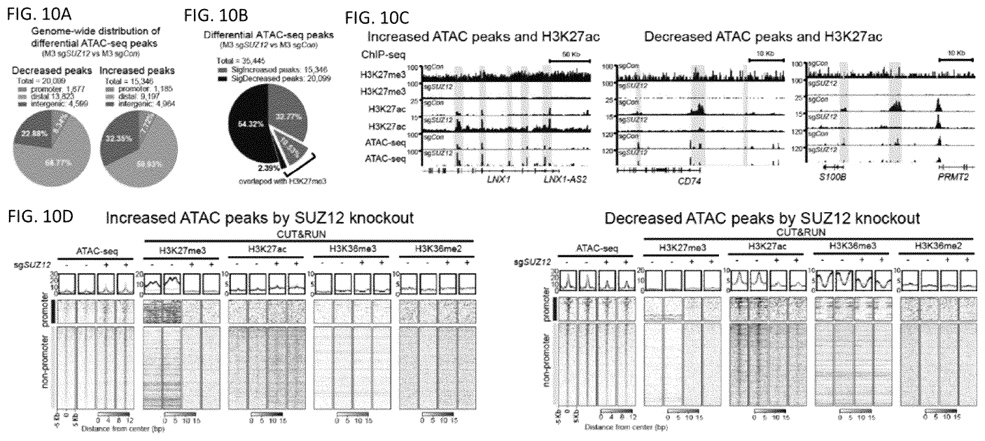

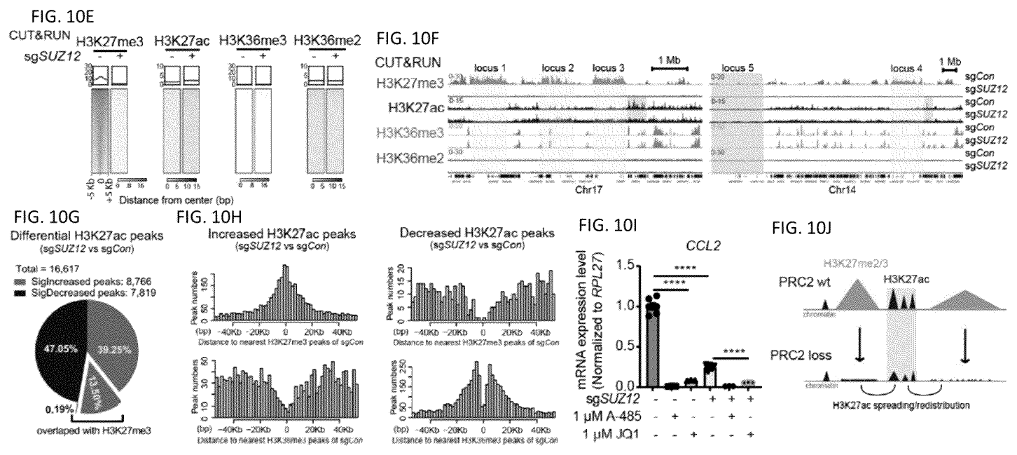

We speculated that PRC2 loss may alter the chromatin context and directly affect the transcriptional regulation of genes related to immune signaling and responses. We first examined the impact of PRC2 loss on genome-wide distribution of chromatin accessibility by ATAC-seq and PRC2 relevant chromatin marks by ChIP-seq and CUT&RUN in PRC2-isogenic M3 cells. Globally, compared to PRC2-wt, PRC2 loss led to not only significant increase, but also significant decrease of chromatin accessibility at 15,346 (16% of all ATAC peaks) and 20,099 genomic loci (21% of all ATAC peaks), respectively (FIG. 3C). The significantly changed chromatin accessibility regions (increased or decreased) were relatively similarly distributed at promoter (7.7%, 8.3%) and non-promoter regions, including distal regulatory (59.9%, 68.8%) and intergenic regions (32.4%, 22.9%) (FIG. 10A). The significantly increased ATAC peaks associated with PRC2 loss overlapped more with H3K27me3 enriched loci (10.5%) than the decreased ATAC peaks (2.4%) (FIG. 10B), consistent with the PRC2 function on chromatin compaction and transcription repression. Importantly, the integration of differential ATAC peaks with multiple histone modifications showed that the increased/decreased H3K27ac peaks were well correlated with open/closed chromatin accessibility changes at both promoter and non-promoter regions as a result of PRC2 loss (FIG. 3D and FIGS. 10C-10D). The global distribution of H3K36me2 and H3K36me3 chromatin marks that had been previously described to interact with PRC2 and H3K27me3 [37-42] were not significantly affected, especially surrounding the H3K27me3-enriched regions in PRC2-wt context (FIGS. 10D-10F).

H3K27ac is an established chromatin mark preferentially enriched at active promoters and distal regulatory enhancers [13, 43, 44]. Despite the increase of total amount of chromatin bound H3K27ac with PRC2 loss, we surprisingly found relatively balanced gains and losses in the genome when regions enriched with H3K27ac were compared (FIG. 3E). While the significantly gained H3K27ac peaks mainly localized to distantly regulatory and intergenic regions and overlapped with H3K27me3 peaks, the significantly lost H3K27ac peaks mainly localized to promoters and existing super enhancers (SE) in PRC2-wt control with minimal overlap with H3K27me3 (FIG. 3E and FIG. 10G). Consistently, the increased H3K27ac peaks in the PRC2/H3K27me loss context displayed a trend of closer distance to nearest H3K27me3 peaks than that of decreased H3K27ac peaks, while their distances to H3K36me3 peaks were reversed (FIG. 1011). Interestingly, we observed that with PRC2/H3K27me loss, a diffuse H3K27ac signal spread into the H3K27me3-enriched regions in the PRC2-wt context without enrichment of specific regions, leading to a mild overall increase of baseline H3K27ac signal (e.g., loci 1-4) (FIGS. 10E-10F). Notably, the significantly decreased H3K27ac peaks localized to genes with higher baseline expression levels compared to those of increased H3K27ac peaks (FIG. 3F). And H3K27ac changes were well correlated with transcriptome changes at both promoter and non-promoter regions (FIG. 3G). Motif analysis demonstrated significant enrichment of transcription factor binding motifs associated with immune signaling pathways and responses, e.g., interferon signaling associated IRF family and ISRE motifs, only in the decreased but not increased H3K27ac peaks associated with PRC2 loss (FIG. 3H and data not shown). In addition, a subset of IFNγ-responsive gene loci was directly affected by PRC2 loss. For example, the H3K27ac enrichment at the super enhancer locus of the monocyte chemotactic protein, CCL2, was significantly diminished by PRC2 loss (sgSUZ12) compared to controls (FIG. 3I), whereas the H3K27ac enrichment at other IFNγ-target gene loci (e.g., IRF1, CD274) was unchanged (FIG. 3J). We observed that many lost and gained super enhancer regions by PRC2 loss were preferentially flanked by broad enrichment of H3K27me3 in the genome of PRC2-wt (FIGS. 3I and 3K). Further, inhibition of the main histone acetyl transferase (CBP/P300) for H3K27ac by A-485, or of the binding of bromodomain (BRD) proteins to H3K27ac by JQ1, suppressed IFNγ-responsive CCL2 gene expression in PRC2-wt M3 cells (FIG. 10I). This data indicated that CCL2 expression in the PRC2-wt context required H3K27ac modification at the CCL2 locus and the diminished expression of CCL2 in the PRC2-loss context was a direct consequence of the decreased H3K27ac enrichment at the CCL2 locus. These data suggest that PRC2 loss affects the global distribution of H3K27ac and reprograms the genome-wide chromatin context of both the promoter and enhancer landscapes, which in turn alters signaling-dependent transcriptional responses.

### Example 5: Decreased Chromatin Accessibility for IFNγ-Responsive Loci in PRC2-Loss MPNST Cells

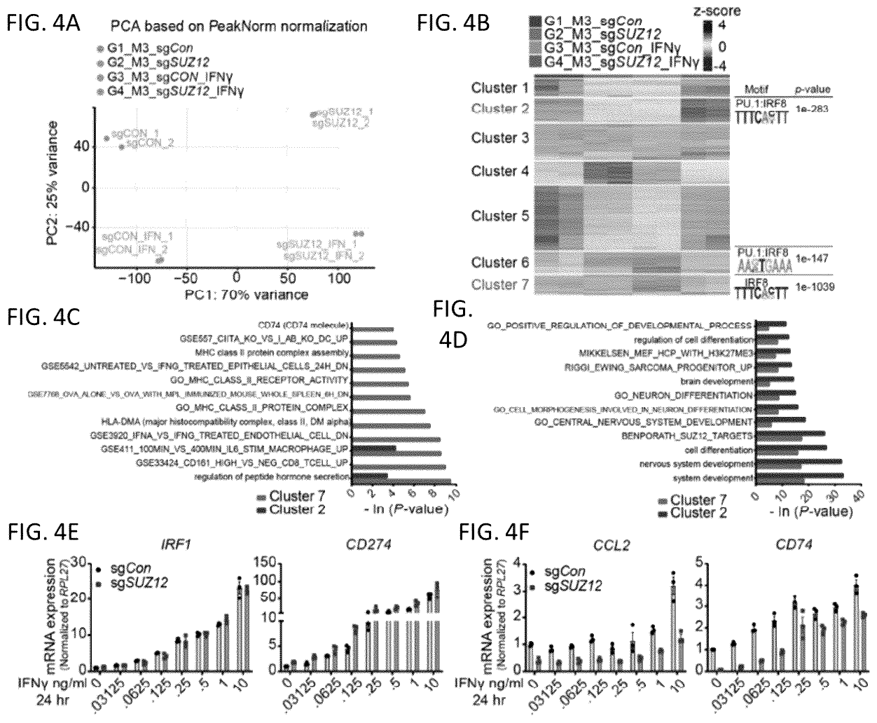

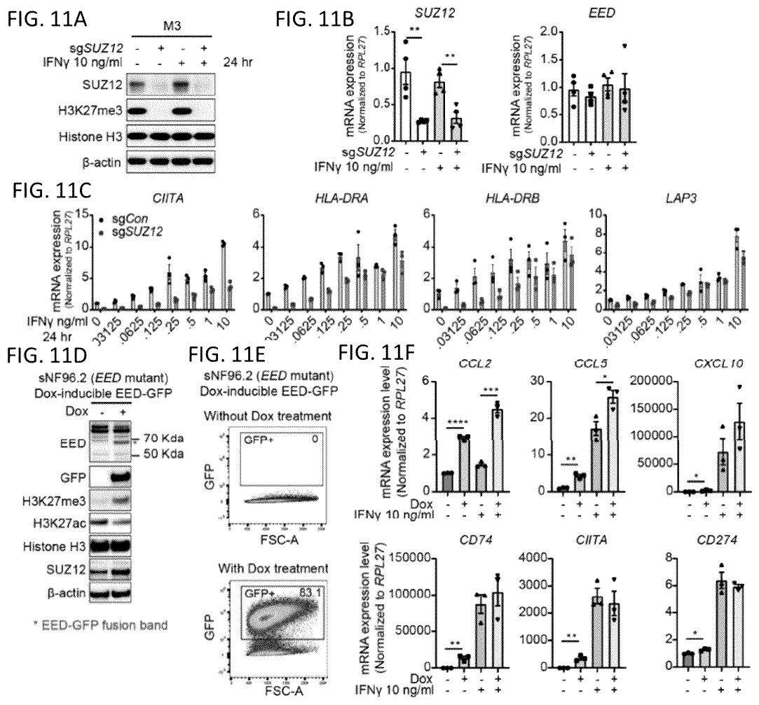

One of the fundamental functions of the chromatin context is to prime the cells for signaling dependent transcriptional response. Reasoning that altered local chromatin context mediated by PRC2 loss may change the transcriptional output, we next examined the transcriptome changes of IFNγ-responsive genes in response to IFNγ stimulation in PRC2-isogenic MPNST cells. Stimulation with 10 ng/ml exogenous IFNγ for 24 hours had no significant impact on the levels of H3K27me3 modification and SUZ12/EED mRNA expression in PRC2-isogenic M3 cells (FIGS. 11A-11B). PCA of ATAC-seq replicates under various conditions demonstrated robust clustering of replicates and separation of samples based on PRC2 status (PCI) and IFNγ stimulation (PC2) (FIG. 4A). K-means clusters 2 and 6/7 were most representative of increased chromatin accessibility in response to IFNγ stimulation in the PRC2-loss (sgSUZ12) and PRC2-wt (sgCon) contexts, respectively (FIG. 4B). Consistently, de novo motif analysis of the K-means clusters showed that only clusters 2, 6 and 7 identified IFNγ stimulation related motifs (e.g., IRF8 and PU.1:IRF8) but with differential significance. The IFNγ-related motif IRF8 is the topmost enriched motif of cluster 7 with a P=1e⁻¹⁰³⁹, consist with a primed chromatin context in response to IFNγ stimulation through IRF activation in the PRC2-wt context. In contrast, the topmost enriched motif of cluster 2 is the GCN4/AP1 motif (P=1e⁻¹²²³), whereas the classic IFNγ signaling relevant motif, IRF8/PU.1 is less significantly enriched (P=1e⁻²⁸³) (FIG. 4B and data not shown), suggesting a shift from IRF signaling to GCN4/AP1 signaling in a PRC2 loss-primed chromatin context. Moreover, GO analysis also revealed significant differences between the PRC2-loss representative cluster 2 and the PRC2-wt representative cluster 7 of chromatin accessibility changes induced by IFNγ. Cluster 7 was most enriched with immune response-related signaling pathways by GO analysis, including antigen presentation and interferon signaling (FIG. 4C), whereas cluster 2 was most enriched with development related pathways, e.g., cellular differentiation, organ development and PRC2 and H3K27me3 targets (FIG. 4D).

Nevertheless, the transcriptome change tracked the changes in local chromatin context. In response to IFNγ stimulation, the transcription of IFNγ-response genes was unchanged when the local chromatin context was unperturbed, e.g., IRF1 and CD274 (FIG. 4E and FIG. 3J); and the transcriptional activation of IFNγ-response genes was significantly blunted (FIG. 4F and FIG. 11C), when the gene loci were associated with a decrease in chromatin accessibility and H3K27ac signal as result of PRC2 loss, e.g., CCL2, CD74, CIITA and HLA-DRA (FIG. 3I and FIG. 10C). Further, the blunted transcriptional activation of these genes was most pronounced when IFNγ source was low (FIG. 4F and FIG. 11C). We also restored EED in an EED-mutant human MPNST cell line sNF96.2 and validated the restoration of PRC2 function by H3K27me3, using a doxycycline-inducible EED system (FIGS. 11D-11E). Re-expression of wild-type EED (PRC2 restoration) in sNF96.2 cells enhanced expression of IFNγ-responsive genes which were otherwise downregulated as a result of PRC2 loss, e.g., CCL2 and CD74 (FIG. 11F). These observations suggest that PRC2 loss reprograms the steady-state primed chromatin context and leads to a blunted IFNγ response in tumor cells.

### Example 6: Engineered PRC2 Loss Recapitulates the Diminished IFNγ Signaling and the Immunosuppressive TME in Both MPNST and Breast Cancer Murine Models

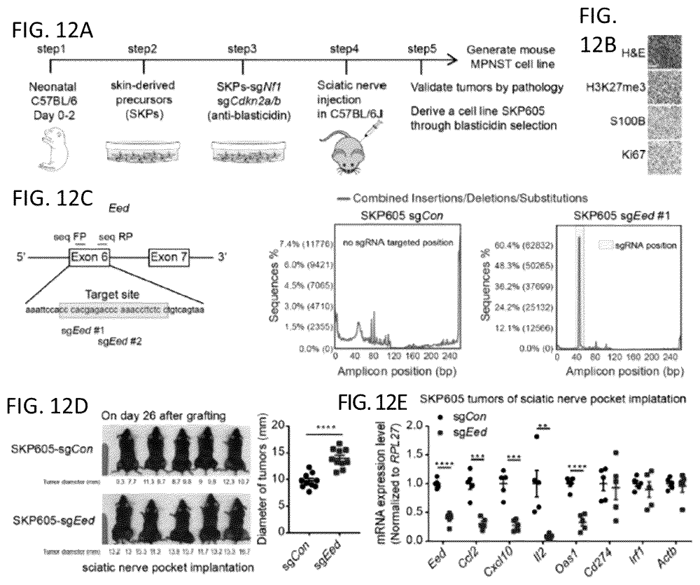

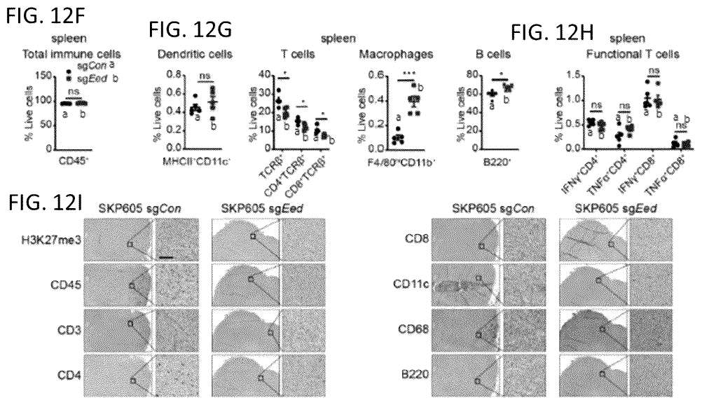

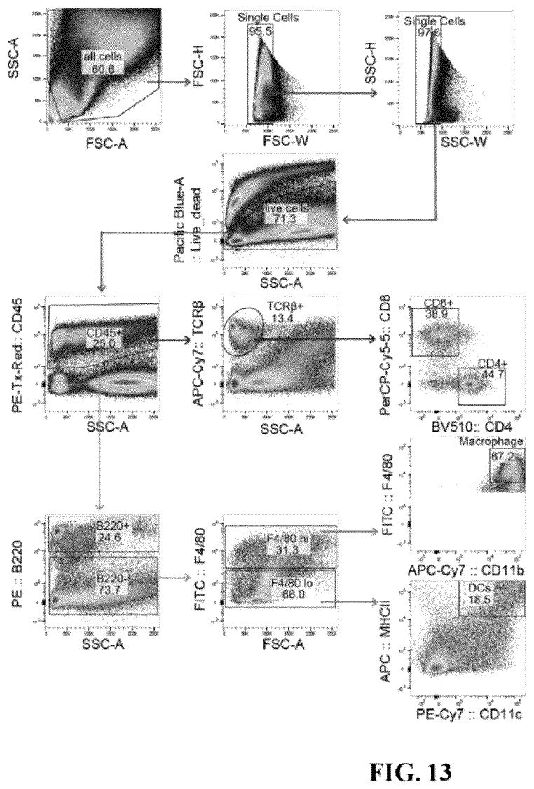

To evaluate the impact of tumor cell-intrinsic PRC2 inactivation on TME in vivo, we generated a histologically confirmed Nf1^{-/-}; Cdkn2a/b^{-/-} murine MPNST tumor-derived cell line (SKP605) from skin-derived precursors (SKPs) of C57BL/6J mice [45, 46] (FIGS. 12A-12B). Using CRISPR/Cas9-mediated knockout of PRC2 core component, Eed, we generated and validated PRC2-isogenic murine MPNST cells (SKP605, sgCon vs. sgEed) amenable for orthotopic and syngeneic transplant in immunocompetent C57BL/6J mice (FIGS. 5A-5B and FIG. 12C). The PRC2-loss (sgEed) orthotopically transplanted tumors exhibited accelerated growth in the sciatic nerve pockets of C57BL/6J mice compared to those of PRC2-wt (sgCon) tumors (FIGS. 5C-5D and FIG. 12D). The expressions of immune cell recruitment chemokines e.g., Ccl2 and Cxcl10, and lymphocyte activation cytokine, 112, were significantly decreased in PRC2-loss tumors compared to PRC2-wt (FIG. 12E), indicating suppressed immune cell infiltration. Profiling of tumor immune infiltrates by FACS demonstrated a significant reduction of CD45⁺ leukocytes in PRC2-loss tumors compared to PRC2-wt (FIG. 5E and FIG. 12F). Further, the reduction of the tumor immune infiltrates in PRC2-loss tumors was across all major subclasses of immune cells, including MHCII⁺CD11c⁺ dendritic cells (DCs), TCRβ⁺ T cells, B220⁺ B cells, and to a lesser extent F4/80^{hi}CD11b⁺ macrophages (FIG. 5F, FIG. 12G, and FIG. 13), phenocopying the TME of human PRC2-loss MPNST. Importantly, the IFN and TNFα⁺CD4⁺ T cells were both significantly reduced in PRC2-loss tumors (FIG. 5G and FIG. 1211). The immune infiltration change between PRC2-isogenic SKP605 tumors was further confirmed by IHC (FIG. 12I). These data suggest that PRC2 inactivation in MPNST causes diminished recruitment of tumor immune infiltrates and functional T cells which contributes to an immunosuppressive TME.

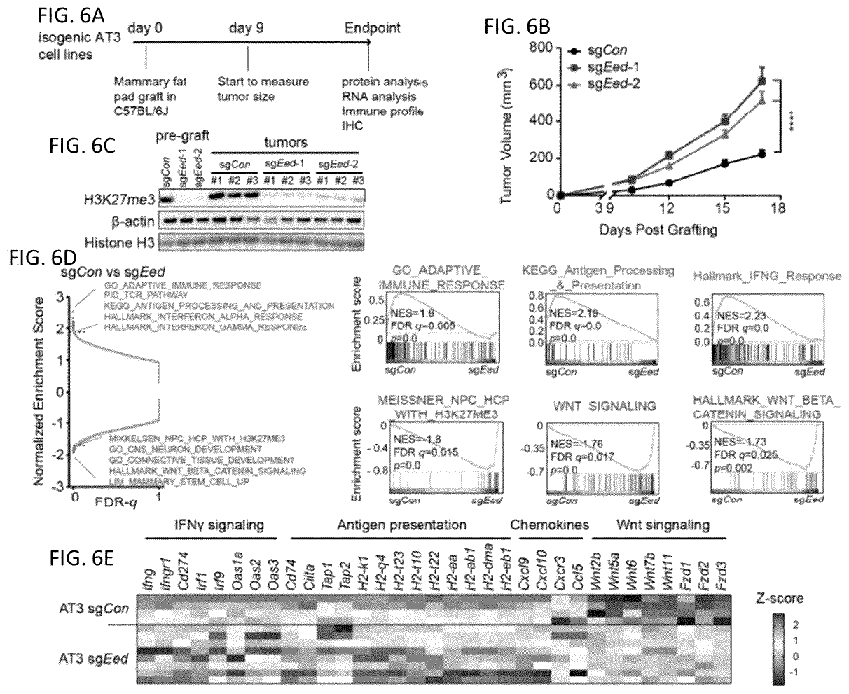

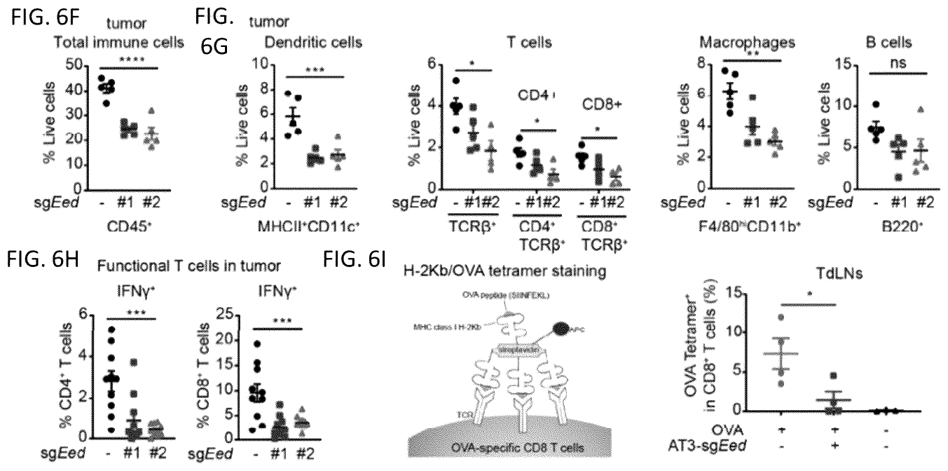

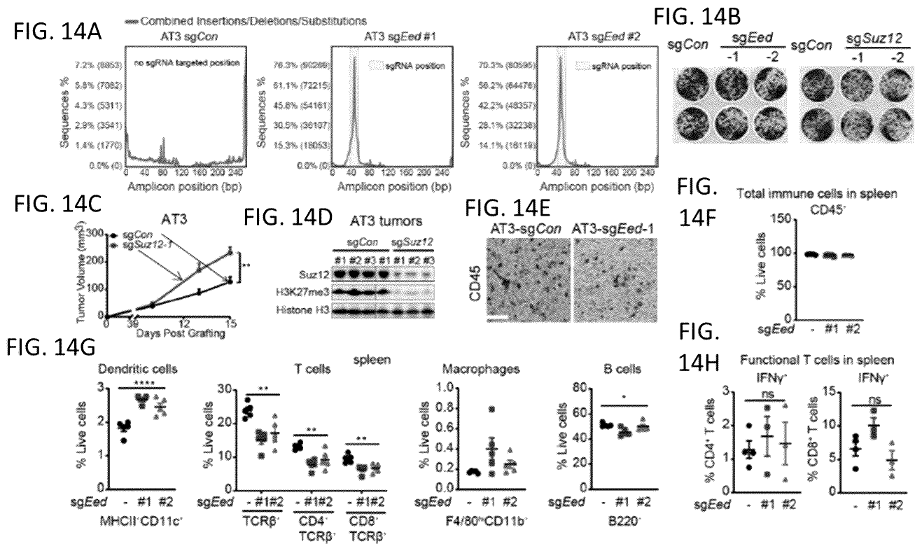

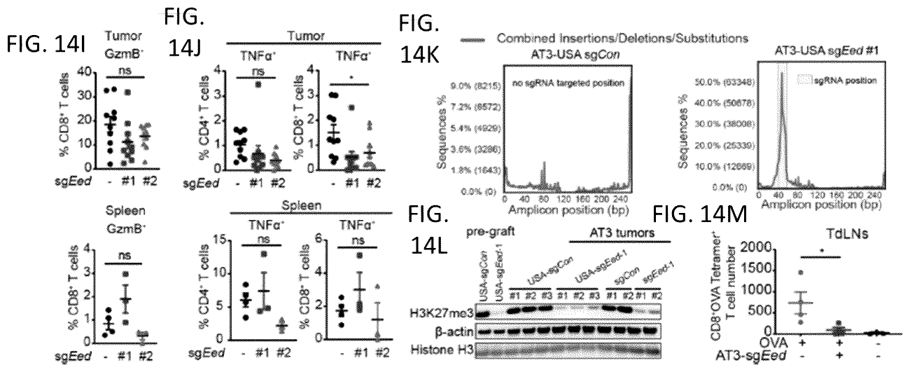

To evaluate whether PRC2 loss exerts similar effect on tumor immune microenvironment in other cancer types, we used CRISPR/Cas9-mediated knockout of PRC2 core components, Eed or Suz12, and generated PRC2-isogenic murine mammary tumor model (AT3, sgCon vs. sgEed or sgSuz12) amenable for syngeneic transplant in C57BL/6J mice (FIG. 14A). Although PRC2 loss did not affect the tumor cell growth in vitro (FIG. 14B), it accelerated orthotopically and syngeneic graft tumor growth in vivo (FIGS. 6A-6B and FIG. 14C). PRC2 downstream target H3K27me3 loss was maintained in grafted PRC2-loss tumors (FIG. 6C and FIG. 14D). Transcriptome analysis of the explanted PRC2-wt (sgCon) and PRC2-loss (sgEed) AT3 tumors by RNA-seq demonstrated that PRC2 loss led to the upregulation of various developmental pathways, including Wnt/β-catenin signaling and PRC2/H3K27me3 targets, and the downregulation of both innate and adaptive immune response pathways, including antigen processing and presentation and IFNγ response pathways (FIG. 6D and data not shown). Consistently, there was significant reduction of the expression of Ifng and Ifnγ signaling-related genes (e.g., Ifngr1, Cd274, Irf1, Irf9), antigen presentation-related genes (e.g., Tap1, Tap2), MHC class I (e.g., H2-k1, H2-q4, H2-23), and MHC class II (e.g., H2-t10 and H2-aa) genes in PRC2-loss tumors compared to PRC2-wt (FIG. 6E), indicating impaired tumor immunogenicity by tumor cell-intrinsic PRC2 inactivation. And there were less immune recruitment chemokines in PRC2-loss tumors (e.g., Cxcl9, Cxcl10, Ccl5). Therefore, engineered PRC2-loss murine mammary tumor phenocopies the transcriptome changes and recapitulates the impaired immunogenicity of PRC2-loss human MPNST, irrespective of tumor lineage.

We next analyzed the population change of infiltrating immune cells in explanted PRC2-isogenic syngeneic transplant tumors (FIG. 13). We observed significant reduction of CD45⁺ leukocytes in PRC2-loss tumors compared to PRC2-wt controls and confirmed by IHC (FIG. 6F and FIGS. 14E-14F). Similarly, the reduction of tumor immune infiltrates by PRC2 loss was across all major subclasses of immune cells, including MHCII⁺CD11c⁺ DCs, TCRβ⁺ T cells (both CD4⁺ and CD8⁺), F4/80^{hi}CD11b⁺ macrophages, and to a lesser extent B220⁺ B cells (FIG. 6G and FIG. 14G). Importantly, functional IFNγ⁺ T cells were significantly reduced in both in CD4⁺ and CD8⁺ T cell subclasses (FIG. 6H and FIG. 14H), as well as a reduction of TNFα⁺ and a trend of reduction of the Granzyme B⁺ (GzmB⁺) CD8⁺ T cells in PRC2-loss tumor (FIGS. 141-14.1). These observations indicate that PRC2 loss in tumor not only lead to diminished numbers of T cells, but also significant functional impairment of T cells. Since functional T cells are the main source of IFNγ, these data indicate that IFNγ is likely significantly diminished in PRC2-loss tumors, which can further amplify the immune evasion phenotype.

To further dissect how PRC2 loss drive an immune evasion phenotype, we specifically evaluated the T cell priming in tumor draining lymph nodes (TdLNs), a critical initial step in the development of anti-tumor immunity [34, 47]. We exogenously expressed the model antigen, ovalbumin (OVA), engineered PRC2-isogenic AT3 OVA+ cells (sgCon vs. sgEed), and orthotopically transplanted these cells in the mammary fat pads of syngeneic C57BL/6J mice, then analyzed the OVA-specific CD8⁺ T cells in the TdLNs (FIGS. 14K-14L). The MHC-I OVA tetramer-positive CD8⁺ T cells were significantly diminished in the TdLNs from mice bearing PRC2-loss OVA⁺ compared to PRC2-wt OVA+AT3 tumors (FIG. 6I and FIG. 14M). These data indicate that PRC2-loss in tumors suppresses the initial antigen cross-presentation by DCs and impairs the tumor-specific CD8⁺ T cell priming. These combined with the decreased tumor-infiltrating DCs and macrophages, as well as diminished immune recruitment chemokines, collectively contribute to the immune-desert TME. The observations from the AT3 murine mammary tumor model also indicate that the PRC2 loss-mediated immune evasion is not restricted to the MPNST context but can be generalized to other cancer contexts.

### Example 7: Engineered PRC2-Loss Tumors Confer Primary Resistance to Immune Checkpoint Blockades

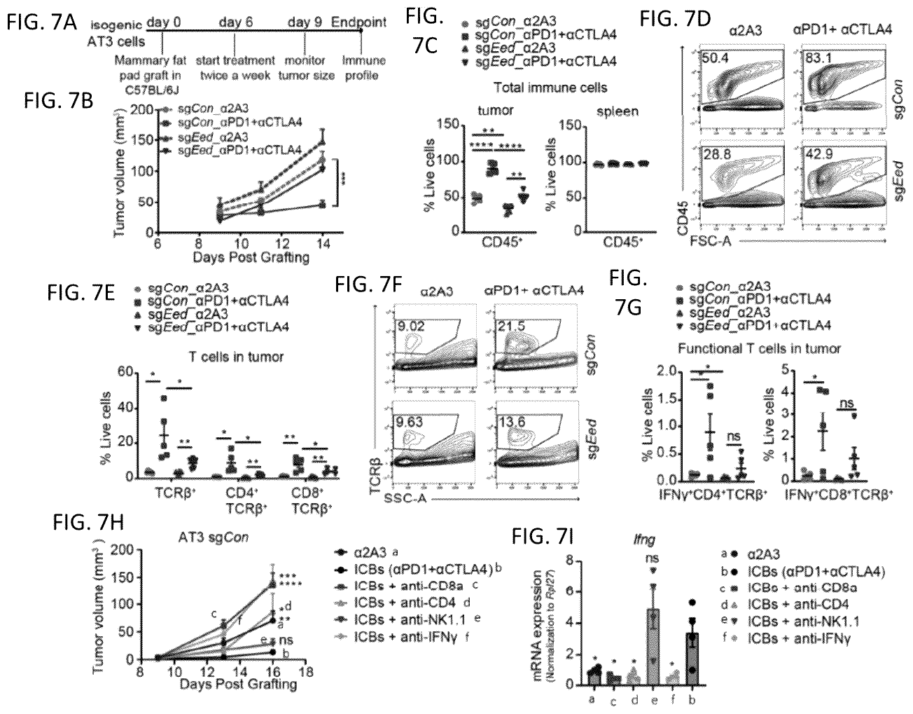

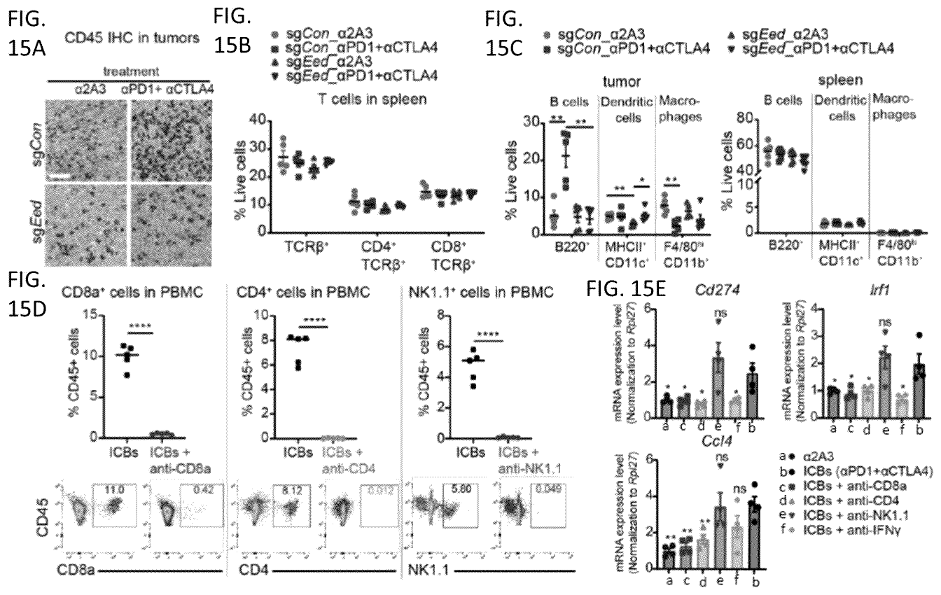

Since tumor cell-intrinsic PRC2 loss drives an immune-desert TME, we speculated that PRC2 loss might confer primary resistance to FDA-approved immune checkpoint blockade (ICB) immunotherapies, including anti-PD1 and anti-CTLA4 antibodies [48]. We evaluated the therapeutic efficacy of combining anti-PD1 and anti-CTLA4 antibodies in transplanted PRC2-isogenic AT3 tumor models (FIG. 7A). Combined ICB was effective and significantly inhibited the growth of PRC2-wt AT3 tumors; in contrast, it failed to retard the growth of PRC2-loss AT3 tumors (FIG. 7B). Moreover, the combined ICB treatment increased CD45⁺ immune infiltrates (FIGS. 7C-7D and FIG. 15A), particularly TCRβ⁺ CD4⁺ and TCRβ⁺CD8⁺ T cell infiltrates in the PRC2-wt tumors (FIGS. 7E-7F and FIGS. 15B-15C). However, the recruitment of CD45⁺ immune cells in response to combined ICB treatment, including both CD4⁺ and CD8⁺ T cells, was significantly blunted in the PRC2-loss tumors; this was accompanied by a reduction of the functional IFNγ⁺CD4 and CD8 T cells (FIGS. 7E-7G). These results demonstrated that PRC2-loss tumors were resistant to ICB therapy.

To evaluate the cellular and molecular components that mediate the ICB treatment responses in PRC2-wt AT3 (sgCon) tumors, we used depletion antibodies to selectively deplete the CD4⁺ T, the CD8⁺ T, or the NK cells, and used IFNγ-blocking antibody to block IFNγ signaling and examined the ICB treatment responses under these perturbations (FIG. 15D). We observed that the NK cell depletion did not significantly affect the ICB treatment response; the CD4⁺ T cell depletion abolished the ICB treatment response; In contrast, IFNγ and CD8⁺ T cell depletion not only diminished ICB treatment responses, but also led to accelerated tumor growth compared to untreated controls (FIG. 71I). Consistently, the expressions of Ifng and Ifnγ-response genes (e.g., Cd274, Irf1, Ccl4) were diminished by depletion of IFNγ, CD4⁺, or CD8⁺ T cells compared to ICB treatment controls (FIG. 7I and FIG. 15E). These data indicate that the IFN in the TME is critical for immune surveillance and control of tumor growth, as well as mediating ICB treatment response; and the source of IFNγ is mainly from CD8⁺ T and partially from CD4⁺ T cells.

### Example 8: Intratumoral Delivery of Immunostimulatory Heat-Inactivated MVA Sensitizes PRC2-Loss Tumors to ICB Therapy

Immunogenic modified vaccinia virus Ankara (MVA) infection of DCs can induce type I IFN by activating the cyclic GMP-AMP synthase (cGAS) and Stimulator of IFN genes (STING)-mediated cytosolic DNA-sensing pathways, e.g., heat-inactivated MVA (heat-iMVA) [49]. Intratumoral (IT) delivery of heat-iMVA can generate local and systemic anti-tumor immunity mediated by CD8⁺ T cells and Batf3-dependent CD103⁺/CD8⁺DCs [50]. We tested whether IT delivery of heat-iMVA can enhance the interferon response and innate immunity, modulate the immune-desert TME, and sensitize the PRC2-loss tumors to ICB. We observed that infecting PRC2-loss murine tumor cells (AT3 [sgEed], SKP605 [sgEed]) with heat-iMVA not only triggered type I IFN (e.g., Ifnb1 and Ifna4) (FIG. 8A), but also rescued the PRC2-loss-mediated IFNγ signaling deficiency, e.g., increased chemokines and Cd274 (Pdl1) expression (FIG. 8B).

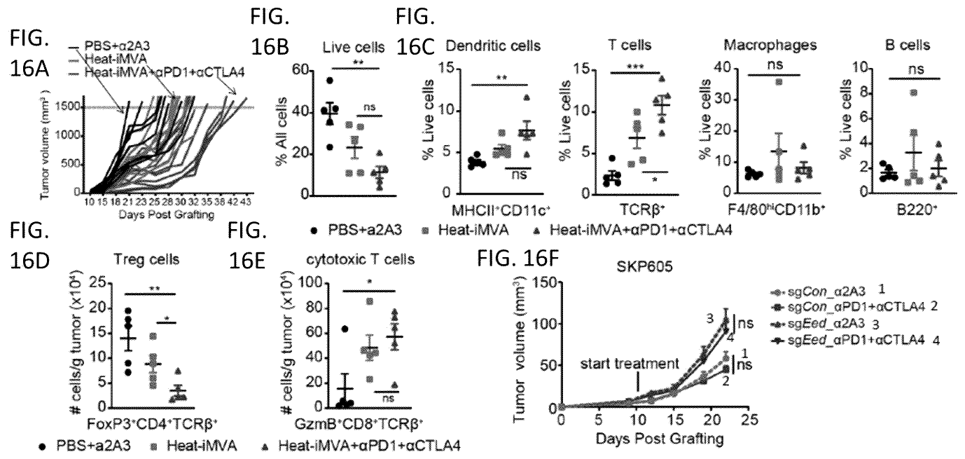

IT heat-iMVA alone in syngeneically transplanted PRC2-loss AT3 (sgEed) tumors only mildly retarded the tumor growth; however, IT heat-iMVA when combined with anti-PD1 and anti-CTLA4 ICB treatment significantly reduced tumor growth (FIGS. 8C-8D and FIG. 16A). This was accompanied by significant prolongation of survival of the heat-iMVA and ICB combination treatment group compared to ICB treatment alone or vehicle groups (FIG. 8E). Consistently, we observed significantly more cell death in PRC2-loss tumors under viral treatment (FIG. 16B), suggesting an enhanced antitumor effect. Consistently, IT heat-iMVA significantly increased CD45⁺ immune cell infiltration in PRC2-loss AT3 (sgEed) tumors compared to vehicle, which was further augmented when combined with ICBs (FIG. 8F), including TCRβ⁺ T cells and MHCII⁺CD11c⁺DCs (FIG. 16C). We did not observe significant changes in F4/80^{hi}CD11b⁺ macrophages and B220⁺ B cells in heat-iMVA injected tumors (FIG. 16C). Both CD4⁺ and CD8⁺ T cells were significantly enriched accompanied by increased proliferation (Ki67⁺) in heat-iMVA treatment group with and without combination with ICB, compared to vehicles (FIG. 8G). Moreover, heat-iMVA treatment led to significant decrease in immune suppressive FoxP3⁺ Treg cells (FIG. 8H and FIG. 16D), and significant increase in GzmB⁺CD8⁺ cytotoxic T cells compared to vehicles (FIG. 8I and FIG. 16E), especially when combined with ICB therapy.

We further investigated IT heat-iMVA in a newly established PRC2-isogenic murine MPNST model (SKP605) amenable for syngeneic transplant that resembles human MPNST (FIG. 5). In the PRC2-wt context, SKP605 (sgCon) tumors already had low levels of tumor immune infiltrates, which were further diminished with PRC2 loss (sgEed) (FIG. 5C); both PRC2-wt and PRC2-loss SKP605 were resistant to ICB treatment (FIG. 16F). Using the murine MPNST tumor model, we observed that IT heat-iMVA sensitized the PRC2-loss SKP605 tumors to ICB treatment and significantly prolonged survival (FIGS. 8J-8K). Notably, 7 of 10 mice treated with the combined IT Heat-iMVA and ICB had complete responses (CR) (FIG. 8J). Moreover, we did not observe any tumor regrowth when the 7 CR mice were rechallenged with SKP605 (sgEed) tumor cells on the opposite side of the previously treated tumor grafts (FIG. 8L), suggesting an adaptive immune response. These results demonstrate that IT delivery of immunogenic MVA therapy combined with ICB is an effective initial strategy to modify the cold TME and elicit anti-tumor effect in PRC2-loss tumors.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1 Margueron, R. and D. Reinberg, The Polycomb complex PRC2 and its mark in life. Nature, 2011. 469(7330): p. 343-9.
2. De Raedt, T., et al., PRC2 loss amplifies Ras-driven transcription and confers sensitivity to BRD4-based therapies. Nature, 2014. 514(7521): p. 247-51.
3. Lee, W., et al., PRC2 is recurrently inactivated through EED or SUZJ2 loss in malignant peripheral nerve sheath tumors. Nat Genet, 2014. 46(11): p. 1227-32.
4. Ernst, T., et al., Inactivating mutations of the histone methyltransferase gene EZH2 in myeloid disorders. Nat Genet, 2010. 42(8): p. 722-6.
5. Nikoloski, G., et al., Somatic mutations of the histone methyltransferase gene EZH2 in myelodysplastic syndromes. Nat Genet, 2010. 42(8): p. 665-7.
6. Ntziachristos, P., et al., Genetic inactivation of the polycomb repressive complex 2 in T cell acute lymphoblastic leukemia. Nat Med, 2012. 18(2): p. 298-301.
7 Zhang, J., et al., The genetic basis of early T-cell precursor acute lymphoblastic leukaemia. Nature, 2012. 481(7380): p. 157-63.
8. Harutyunyan, A. S., et al., H3K27M induces defective chromatin spread of PRC2-mediated repressive H3K27me2/me3 and is essential for glioma tumorigenesis. Nat Commun, 2019. 10(1): p. 1262.
9. Lewis, P. W., et al., Inhibition of PRC2 activity by a gain-of-function H3 mutation found in pediatric glioblastoma. Science, 2013. 340(6134): p. 857-61.
10. Lulla, R. R., A. M. Saratsis, and R. Hashizume, Mutations in chromatin machinery and pediatric high-grade glioma. Sci Adv, 2016. 2(3): p. e1501354.
11. Stafford, J. M., et al., Multiple modes of PRC2 inhibition elicit global chromatin alterations in H3K27M pediatric glioma. Sci Adv, 2018. 4(10): p. eaau5935.
12. Wassef, M., et al., Impaired PRC2 activity promotes transcriptional instability and favors breast tumorigenesis. Genes Dev, 2015. 29(24): p. 2547-62.
13. Prieto-Granada, C. N., et al., Loss of H3K27me3 Expression Is a Highly Sensitive Marker for Sporadic and Radiation-induced MPNST. Am J Surg Pathol, 2016. 40(4): p. 479-89.
14. Schaefer, I. M., C. D. Fletcher, and J. L. Hornick, Loss of H3K27 trimethylation distinguishes malignant peripheral nerve sheath tumors from histologic mimics. Mod Pathol, 2016. 29(1): p. 4-13.
15. Zhang, M., et al., Somatic mutations of SUZ12 in malignant peripheral nerve sheath tumors. Nat Genet, 2014. 46(11): p. 1170-2.
16. Chen, G., et al., Ezh2 Regulates Activation-Induced CD8(+) T Cell Cycle Progression via Repressing Cdkn2a and Cdkn1c Expression. Front Immunol, 2018. 9: p. 549.
17. Gray, S. M., et al., Polycomb Repressive Complex 2-Mediated Chromatin Repression Guides Effector CD8(+) T Cell Terminal Differentiation and Loss of Multipotency. Immunity, 2017. 46(4): p. 596-608.
18. He, S., et al., Ezh2 phosphorylation state determines its capacity to maintain CD8(+) T memory precursors for antitumor immunity. Nat Commun, 2017. 8(1): p. 2125.
19. Kakaradov, B., et al., Early transcriptional and epigenetic regulation of CD8(+) T cell differentiation revealed by single-cell RNA sequencing. Nat Immunol, 2017. 18(4): p. 422-432.
20. Russ, B. E., et al., Distinct epigenetic signatures delineate transcriptional programs during virus-specific CD8(+) T cell differentiation. Immunity, 2014. 41(5): p. 853-65.
21. Zhao, E., et al., Cancer mediates effector T cell dysfunction by targeting microRNAs and EZH2 via glycolysis restriction. Nat Immunol, 2016. 17(1): p. 95-103.
22. Zhang, Y., et al., The polycomb repressive complex 2 governs life and death of peripheral T cells. Blood, 2014. 124(5): p. 737-49.
23. Yang, X. P., et al., EZH2 is crucial for both differentiation of regulatory T cells and T effector cell expansion. Sci Rep, 2015. 5: p. 10643.
24. Tumes, D. J., et al., The polycomb protein Ezh2 regulates differentiation and plasticity of CD4(+) T helper type 1 and type 2 cells. Immunity, 2013. 39(5): p. 819-32.
25. De Simone, M., et al., Transcriptional Landscape of Human Tissue Lymphocytes Unveils Uniqueness of Tumor-Infiltrating T Regulatory Cells. Immunity, 2016. 45(5): p. 1135-1147.
26. DuPage, M., et al., The chromatin-modifying enzyme Ezh2 is critical for the maintenance of regulatory T cell identity after activation. Immunity, 2015. 42(2): p. 227-238.
27. Wang, D., et al., Targeting EZH2 Reprograms Intratumoral Regulatory T Cells to Enhance Cancer Immunity. Cell Rep, 2018. 23(11): p. 3262-3274.
28. Peng, D., et al., Epigenetic silencing of TH I-type chemokines shapes tumour immunity and immunotherapy. Nature, 2015. 527(7577): p. 249-53.
29. Burr, M. L., et al., An Evolutionarily Conserved Function of Polycomb Silences the MHC Class I Antigen Presentation Pathway and Enables Immune Evasion in Cancer. Cancer Cell, 2019. 36(4): p. 385-401 e8.
30. Won, H. H., et al., Detecting somatic genetic alterations in tumor specimens by exon capture and massively parallel sequencing. J Vis Exp, 2013(80): p. e50710.
31. Huang, X., et al., Targeting Epigenetic Crosstalk as a Therapeutic Strategy for EZH2-Aberrant Solid Tumors. Cell, 2018. 175(1): p. 186-199 e19.
32. Kim, J., et al., A Myc network accounts for similarities between embryonic stem and cancer cell transcription programs. Cell, 2010. 143(2): p. 313-24.
33. Mikkelsen, T. S., et al., Genome-wide maps of chromatin state in pluripotent and lineage-committed cells. Nature, 2007. 448(7153): p. 553-60.
34. Chen, D. S. and I. Mellman, Oncology meets immunology: the cancer-immunity cycle.

Immunity, 2013. 39(1): p. 1-10.
35. Dunn, G. P., C. M. Koebel, and R. D. Schreiber, Interferons, immunity and cancer immunoediting. Nat Rev Immunol, 2006. 6(11): p. 836-48.
36. Parker, B. S., J. Rautela, and P. J. Hertzog, Antitumour actions of interferons: implications for cancer therapy. Nat Rev Cancer, 2016. 16(3): p. 131-44.
37. Li, J., J. H. Ahn, and G. G. Wang, Understanding histone H3 lysine 36 methylation and its deregulation in disease. Cell Mol Life Sci, 2019. 76(15): p. 2899-2916.
38. Lu, C., et al., Histone H3K36 mutations promote sarcomagenesis through altered histone methylation landscape. Science, 2016. 352(6287): p. 844-9.
39. Miyazaki, H., et al., Ash11 methylates Lys36 of histone H3 independently of transcriptional elongation to counteract polycomb silencing. PLoS Genet, 2013. 9(11): p. e1003897.
40. Popovic, R., et al., Histone methyltransferase MMSET/NSD2 alters EZH2 binding and reprograms the myeloma epigenome through global and focal changes in H3K36 and H3K27 methylation. PLoS Genet, 2014. 10(9): p. e1004566.
41. Streubel, G., et al., The H3K36me2 Methyltransferase Nsd1 Demarcates PRC2-Mediated H3K27me2 and H3K27me3 Domains in Embryonic Stem Cells. Mol Cell, 2018. 70(2): p. 371-379 e5.
42. Zhao, S., C. D. Allis, and G. G. Wang, The language of chromatin modification in human cancers. Nat Rev Cancer, 2021. 21(7): p. 413-430.
43. Heintzman, N. D., et al., Distinct and predictive chromatin signatures of transcriptional promoters and enhancers in the human genome. Nat Genet, 2007. 39(3): p. 311-8.
44. Roadmap Epigenomics, C., et al., Integrative analysis of 111 reference human epigenomes. Nature, 2015. 518(7539): p. 317-30.
45. Chau, V., et al., Preclinical therapeutic efficacy of a novel pharmacologic inducer of apoptosis in malignant peripheral nerve sheath tumors. Cancer Res, 2014. 74(2): p. 586-97.
46. Le, L. Q., et al., Cell of origin and microenvironment contribution for NF1-associated dermal neurofibromas. Cell Stem Cell, 2009. 4(5): p. 453-63.
47. Spranger, S. and T. F. Gajewski, Impact of oncogenic pathways on evasion of antitumour immune responses. Nat Rev Cancer, 2018. 18(3): p. 139-147.
48. Sanmamed, M. F. and L. Chen, A Paradigm Shift in Cancer Immunotherapy: From Enhancement to Normalization. Cell, 2018. 175(2): p. 313-326.
49. Dai, P., et al., Modified vaccinia virus Ankara triggers type I IFN production in murine conventional dendritic cells via a cGAS/STING-mediated cytosolic DNA-sensing pathway. PLoS Pathog, 2014. 10(4): p. e1003989.
50. Dai, P., et al., Intratumoral delivery of inactivated modified vaccinia virus Ankara (iMVA) induces systemic antitumor immunity via STING and Batf3-dependent dendritic cells. Sci Immunol, 2017. 2(11).
51. Casey, S. C., et al., MYC regulates the antitumor immune response through CD47 and PD-L1. Science, 2016. 352(6282): p. 227-31.
52. Iannello, A., et al., p53-dependent chemokine production by senescent tumor cells supports NKG2D-dependent tumor elimination by natural killer cells. J Exp Med, 2013. 210(10): p. 2057-69.
53. Koyama, S., et al., STK11/LKB1 Deficiency Promotes Neutrophil Recruitment and Proinflammatory Cytokine Production to Suppress T-cell Activity in the Lung Tumor Microenvironment. Cancer Res, 2016. 76(5): p. 999-1008.
54. Peng, W., et al., Loss of PTEN Promotes Resistance to T Cell-Mediated Immunotherapy. Cancer Discov, 2016. 6(2): p. 202-16.
55. Rakhra, K., et al., CD4(+) T cells contribute to the remodeling of the microenvironment required for sustained tumor regression upon oncogene inactivation. Cancer Cell, 2010. 18(5): p. 485-98.
56. Sai, J., et al., PI3K Inhibition Reduces Mammary Tumor Growth and Facilitates Antitumor Immunity and Anti-PD1 Responses. Clin Cancer Res, 2017. 23(13): p. 3371-3384.
57. Seiwert, T. Y., et al., Integrative and comparative genomic analysis of HPV positive and HPV-negative head and neck squamous cell carcinomas. Clin Cancer Res, 2015. 21(3): p. 632-41.
58. Spranger, S., R. Bao, and T. F. Gajewski, Melanoma-intrinsic beta-catenin signalling prevents anti-tumour immunity. Nature, 2015. 523(7559): p. 231-5.
59. Stine, Z. E., et al., MYC, Metabolism, and Cancer. Cancer Discov, 2015. 5(10): p. 1024-39.
60. Wojcik, J. B., et al., Epigenomic Reordering Induced by Polycomb Loss Drives Oncogenesis but Leads to Therapeutic Vulnerabilities in Malignant Peripheral Nerve Sheath Tumors. Cancer Res, 2019. 79(13): p. 3205-3219.
61. Spranger, S., et al., Tumor Residing Batf3 Dendritic Cells Are Required for Effector T Cell Trafficking and Adoptive T Cell Therapy. Cancer Cell, 2017. 31(5): p. 711-723 e4.
62. Miao, D., et al., Genomic correlates of response to immune checkpoint therapies in clear cell renal cell carcinoma. Science, 2018. 359(6377): p. 801-806.
63. Pan, D., et al., A major chromatin regulator determines resistance of tumor cells to T cell-mediated killing. Science, 2018. 359(6377): p. 770-775.
64. Bommareddy, P. K., M. Shettigar, and H. L. Kaufman, Integrating oncolytic viruses in combination cancer immunotherapy. Nat Rev Immunol, 2018. 18(8): p. 498-513.
65. Russell, S. J. and G. N. Barber, Oncolytic Viruses as Antigen Agnostic Cancer Vaccines. Cancer Cell, 2018. 33(4): p. 599-605.
66. Pittman, P. R., et al., Phase 3 Efficacy Trial of Modified Vaccinia Ankara as a Vaccine against Smallpox. N Engl J Med, 2019. 381(20): p. 1897-1908.
67. Volz, A. and G. Sutter, Modified Vaccinia Virus Ankara: History, Value in Basic Research, and Current Perspectives for Vaccine Development. Adv Virus Res, 2017. 97: p. 187-243.
68. Dobin, A., et al., STAR: ultrafast universal RNA-seq aligner. Bioinformatics, 2013. 29(1): p. 15-21.
69. Trapnell, C., et al., Transcript assembly and quantification by RNA-Seq reveals unannotated transcripts and isoform switching during cell differentiation. Nat Biotechnol, 2010. 28(5): p. 511-5.
70. Subramanian, A., et al., Gene set enrichment analysis: a knowledge-based approach for interpreting genome-wide expression profiles. Proc Natl Acad Sci USA, 2005. 102(43): p. 15545-50.
71. Buenrostro, J. D., et al., Transposition of native chromatin for fast and sensitive epigenomic profiling of open chromatin, DNA-binding proteins and nucleosome position. Nat Methods, 2013. 10(12): p. 1213-8.
72. Buenrostro, J. D., et al., ATAC-seq: A Method for Assaying Chromatin Accessibility Genome-Wide. Curr Protoc Mol Biol, 2015. 109: p. 21 29 1-21 29 9.
73. Chi, P., et al., ETV1 is a lineage survival factor that cooperates with KIT in gastrointestinal stromal tumours. Nature, 2010. 467(7317): p. 849-53.
74. Langmead, B., et al., Ultrafast and memory-efficient alignment of short DNA sequences to the human genome. Genome Biol, 2009. 10(3): p. R25.
75. Zhang, Y., et al., Model-based analysis of ChIP-Seq (MACS). Genome Biol, 2008. 9(9): p. R137.
76. Consortium, E. P., An integrated encyclopedia of DNA elements in the human genome. Nature, 2012. 489(7414): p. 57-74.
77. Carroll, T. S., et al., Impact of artifact removal on ChIP quality metrics in ChIP-seq and ChIP-exo data. Front Genet, 2014. 5: p. 75.
78. Banaszynski, L. A., et al., Hira-dependent histone H3.3 deposition facilitates PRC2 recruitment at developmental loci in ES cells. Cell, 2013. 155(1): p. 107-20.
79. Whyte, W. A., et al., Master transcription factors and mediator establish super-enhancers at key cell identity genes. Cell, 2013. 153(2): p. 307-19.
80. Loven, J., et al., Selective inhibition of tumor oncogenes by disruption of super-enhancers. Cell, 2013. 153(2): p. 320-34.
81. Heinz, S., et al., Simple combinations of lineage-determining transcription factors prime cis-regulatory elements required for macrophage and B cell identities. Mol Cell, 2010. 38(4): p. 576-89.

What is claimed is:

1. A method for selecting a cancer patient for treatment with an immune checkpoint inhibitor comprising
(a) detecting the presence of at least one mutation that results in reduced expression or activity of Polycomb Repressive Complex 2 (PRC2) in a biological sample obtained from the cancer patient; and
(b) administering to the cancer patient an effective amount of inactivated modified vaccinia virus Ankara (MVA) or MVA ΔE3L and an effective amount of an immune checkpoint inhibitor.

2. The method of claim 1, wherein the at least one mutation comprises a mutation in SUZ12, EED, and/or EZH1/2.

3. The method of claim 2, wherein the at least one mutation is a nonsense mutation, a missense mutation, a deletion, an inversion or a frameshift mutation.

4. The method of claim 1, wherein the at least one mutation is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

5. A method for treating cancer in a patient in need thereof comprising
administering to the patient an effective amount of inactivated modified vaccinia virus Ankara (MVA) or MVA ΔE3L and an effective amount of an immune checkpoint inhibitor,
wherein mRNA and/or polypeptide expression and/or activity levels of PRC2 in a biological sample obtained from the patient are reduced compared to a control sample obtained from a healthy subject or a predetermined threshold.

6. The method of claim 5, wherein mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

7. The method of claim 5, wherein polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

8. The method of claim 1, wherein the biological sample obtained from the cancer patient comprises biopsied tumor tissue, whole blood, plasma, or serum.

9. A method for sensitizing PRC2 mutant tumors to treatment with an immune checkpoint inhibitor in a patient in need thereof comprising administering to the patient an effective amount of inactivated modified vaccinia virus Ankara (MVA) or MVA ΔE3L separately, sequentially or simultaneously with the immune checkpoint inhibitor.

10. The method of claim 5, wherein inactivation of MVA or MVA ΔE3L occurs via heat-induced inactivation or UV radiation-induced inactivation.

11. The method of claim 5, wherein the immune checkpoint inhibitor comprises one or more of a PD-1/PD-L1 inhibitor, a CTLA-4 inhibitor, pembrolizumab, nivolumab, cemiplimab, atezolizumab, avelumab, durvalumab, ipilimumab, tremelimumab, ticlimumab, JTX-4014, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, AMP-514, KN035, CK-301, AUNP12, CA-170, or BMS-986189.

12. The method of claim 5, wherein the patient suffers from or is diagnosed with malignant peripheral nerve sheath tumor (MPNST), melanoma, a myeloid disorder, T-cell acute lymphocytic leukemia (ALL), early T-cell precursor ALL, pediatric glioma, or invasive breast cancer.

13. The method of claim 9, wherein the immune checkpoint inhibitor is administered orally, intranasally, parenterally, intravenously, intramuscularly, intraperitoneally, subcutaneously, rectally, intrathecally, intratumorally or topically.

14. The method of claim 9, wherein the inactivated MVA or MVA ΔE3L is administered orally, intranasally, parenterally, intravenously, intramuscularly, intraperitoneally, subcutaneously, rectally, intrathecally, intratumorally or topically.
